(19) United States
(12) Patent Application Publication
(10) Pub. No.: US 2006/0021082 A1
(43) Pub. Date: Jan. 26, 2006

(54) Cytokinin oxidase sequences and methods of use

(75) Inventors: Norbert Brugiere, Johnston, IA (US); Jeffrey E. Habben, Urbandale, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International, Inc.

(21) Appl. No.: 11/094,917

(22) Filed: Mar. 31, 2005

Related U.S. Application Data
(60) Provisional application No. 60/559,252, filed on Apr. 2, 2004.

(51) Int. Cl. A01H 1/00; A01H 5/00; C12N 5/04; C07H 21/04; C12N 9/06

(52) U.S. Cl. 800/278; 800/320.1; 435/191; 435/412; 536/23.2

(57) ABSTRACT

Methods and compositions for modulating plant development are provided. Polynucleotide sequences and amino acid sequences encoding cytokinin oxidase polypeptides are provided. The sequences can be used in a variety of methods including modulating root development, modulating floral development, modulating leaf and/or shoot development, modulating seed size and/or weight, modulating tolerance under abiotic stress, and modulating resistance to pathogens. Polynucleotides comprising CKX promoters are also provided. The promoters can be used to regulate expression of a sequence of interest. Transformed plants, plant cells, tissues, and seed are also provided.

This application claims the benefit of, and hereby incorporates by reference, provisional application 60/559,252 filed Apr. 2, 2004.

### FIELD OF THE INVENTION

The invention relates to the field of the genetic manipulation of plants, particularly the modulation of gene activity and development in plants.

### BACKGROUND OF THE INVENTION

Cytokinins are a class of N⁶ substituted purine derivative plant hormones that regulate cell division, as well as a large number of developmental events, such as shoot development, root branching, control of apical dominance in the shoot, leaf development, chloroplast development, and leaf senescence (Mok et al. (1994) Cytokinins. Chemistry, Action and Function. CRC Press, Boca Raton, Fla., pp. 155-166).

The catabolic enzyme cytokinin oxidase (CKX) plays a major role in controlling cytokinin levels in plant tissues, and CKX activity has been found in a great number of plant tissues. The CKX enzyme is a FAD-containing oxidoreductase that catalyzes the degradation of cytokinins bearing unsaturated isoprenoid side chains. The CKX enzymes irreversibly inactivate most cytokinins by cleaving the isoprenoid side chain from the adenine ring (Armstrong et al. (1994). Cytokinins. Chemistry, Action and Function. CRC Press, Boca Raton, Fla., pp. 139-154).

In view of the influence of cytokinins on a wide variety of plant developmental processes, including root architecture, shoot and leaf development, and seed set, the ability to manipulate cytokinin levels in higher plant cells, and thereby affect plant growth and productivity, is of great commercial value.

### BRIEF SUMMARY OF THE INVENTION

Compositions of the invention include cytokinin oxidase (CKX) polypeptides and polynucleotides that are involved in modulating plant development, morphology, and physiology. Compositions include isolated polypeptides comprising an amino acid sequence selected from the group consisting of: (a) the amino acid sequence comprising SEQ ID NO:3, 6, 10, 14, or 53; (b) the amino acid sequence comprising at least 60% sequence identity to SEQ ID NO:3, 6, 10, 14, or 53, wherein said polypeptide has cytokinin oxidase activity; (c) the amino acid sequence encoded by a nucleotide sequence that hybridizes under stringent conditions to the complement of SEQ ID NO:2, 5, 9, 11, 54, or 55, wherein said stringent conditions comprise hybridization in 50% formamide, 1 M NaCl, 1% SDS at 37° C., and a wash in 0.1×SSC at 60° C. to 65° C.; and, (d) the amino acid sequence comprising at least 20 consecutive amino acids of SEQ ID NO:3, 6, 10, 14, or 53, wherein said polypeptide retains cytokinin oxidase activity.

Compositions further include isolated polynucleotides comprising a nucleotide sequence selected from the group consisting of: (a) a nucleotide sequence comprising SEQ ID NO:1, 2, 4, 5, 7, 8, 10, 11, 51, 52, 54, or 55; (b) a nucleotide sequence encoding an amino acid sequence comprising SEQ ID NO:3, 6, 10, 14, or 53; (c) a nucleotide sequence comprising at least 60% sequence identity to SEQ ID NO: 1, 2, 4, 5, 7, 8, 10, 11, 51, 52, 54, or 55, wherein said polynucleotide encodes a polypeptide having cytokinin oxidase activity; (d) a nucleotide sequence comprising at least 20 consecutive nucleotides of SEQ ID NO: 1, 2, 4, 5, 7, 8, 10, 11, 51, 52, 54, or 55 or a complement thereof; and, (e) a nucleotide sequence that hybridizes under stringent conditions to the complement of a nucleotide sequence of a), wherein said stringent conditions comprise hybridization in 50% formamide, 1 M NaCl, 1% SDS at 37° C., and a wash in 0.1×SSC at 60° C. to 65° C.

Compositions also include plants comprising a CKX polypeptide of the invention operably linked to a promoter that drives expression in the plant. The plants of the invention can have a modulated cytokinin level compared to a control plant. In some plants, the cytokinin level is modulated in a vegetative tissue, a reproductive tissue, or a vegetative tissue and a reproductive tissue. Plants of the invention may have at least one of the following phenotypes: modulated floral development, modulated flowering time, modulated root development, an altered shoot-to-root ratio, increased seed size and/or increased seed weight, increased plant yield and/or plant vigor, improved or maintained stress tolerance, or a decrease in shoot growth, when compared to a control plant.

Compositions further include plants that have been genetically modified at a genomic locus, wherein the genomic locus encodes a CKX polypeptide of the invention.

Methods for increasing the level or activity of a CKX polypeptide in a plant are provided, which may decrease the level of cytokinin in the plant. The method can comprise introducing into the plant a CKX polynucleotide of the invention. In certain methods, the activity of the CKX polypeptide is increased in a vegetative tissue, a reproductive tissue, or a vegetative tissue and a reproductive tissue. In certain embodiments, increasing the activity of the CKX polypeptide modulates root development, alters the shoot-to-root ratio, and/or modulates floral development.

Methods for reducing or eliminating the level of a CKX polypeptide in a plant are also provided. The method can comprise introducing into said plant a CKX polynucleotide of the invention using techniques to result in downregulation. Reducing the level or activity of the CKX polypeptide can increase the level of a cytokinin in the plant. The level or activity of the polypeptide is reduced or eliminated in a vegetative tissue, a reproductive tissue, or a vegetative tissue and a reproductive tissue. In other methods, reducing the level and/or activity of the CKX polypeptide maintains or improves the stress tolerance of the plant, increases seed size and/or seed weight, increases the shoot growth of the plant, and/or delays leaf senescence.

Methods and compositions for regulating gene expression in a plant are also provided. Polynucleotides comprising promoter sequences are provided. Compositions include isolated polynucleotides comprising a nucleotide sequence selected from the group consisting of: (a) a nucleotide sequence comprising SEQ ID NO:15, 16, 17, or 18; (b) a nucleotide sequence comprising at least 60% sequence identity to SEQ ID NO:15, 16, 17, or 18, wherein said polynucleotide retains the ability to regulate transcription; (c) a nucleotide sequence comprising at least 20 consecutive nucleotides of SEQ ID NO: 15, 16, 17, or 18, wherein said polynucleotide retains the ability to regulate transcription; and, (d) a nucleotide sequence that hybridizes under stringent conditions to the complement of the nucleotide sequence of a), wherein said stringent conditions comprise hybridization in 50% formamide, 1 M NaCl, 1% SDS at 37° C., and a wash in 0.1×SSC at 60° C. to 65° C., wherein said sequence retains the ability to regulate transcription. Compositions further include plants and seed having a DNA construct comprising a nucleotide sequence of interest operably linked to a CKX promoter of the invention. In specific embodiments, the DNA construct is stably integrated into the genome of the plant.

Methods for regulating the expression of a nucleotide sequence of interest are also provided. The method comprises introducing into a plant a nucleotide sequence of interest operably linked to a CKX promoter of the invention.

### BRIEF DESCRIPTION OF THE DRAWINGS

FIG. 1A-C provides an amino acid alignment of ZmCkx1 (SEQ ID NO:33), ZmCkx2 (SEQ ID NO:3), ZmCkx3 (SEQ ID NO:6), ZmCkx4 (SEQ ID NO:9), ZmCkx5 (SEQ ID NO:12), and ZmCkx6 (SEQ ID NO: 53). A consensus sequence is also provided (SEQ ID NO:34). The alignment was generated with AlignX from the VNTI suite using the blosum62mt2 matrix, a gap opening penalty of 10 and gap extension penalty of 0.05, a gap separation penalty range of 8 and a % identity for alignment delay of 40.

FIG. 2A-F provides an amino acid alignment of AtCkx1 (SEQ ID NO:35), AtCkx2 (SEQ ID NO:36), AtCkx3 (SEQ ID NO:37), AtCkx4 (SEQ ID NO:38), AtCkx5 (SEQ ID NO:39), AtCkx6 (SEQ ID NO:40), AtCkx7 (SEQ ID NO:41), DsCkx1 (SEQ ID NO:42), HvCkx2 (SEQ ID NO:43), HvCkx3 (SEQ ID NO:44), OsCkx1 (SEQ ID NO:45), OsCkx2 (SEQ ID NO:46), OsCkx3 (SEQ ID NO:47), OsCkx4 (SEQ ID NO:48), OsCkx5 (SEQ ID NO:49), ZmCkx1 (SEQ ID NO:33), ZmCkx2 (SEQ ID NO:3), ZmCkx3 (SEQ ID NO:6), ZmCkx4 (SEQ ID NO:10) and ZmCkx5 (SEQ ID NO:14). A consensus sequence is provided in SEQ ID NO:50. The alignment was performed using Clustal W.

FIG. 3 provides a summary of the expression profile for ZmCkx2 in different maize tissues using Pioneer's Lynx database. The highest levels of expression of ZmCkx2 are found in leaves, stalk, whorl, roots and seedlings.

FIG. 4 provides an analysis of a proprietary Lynx database for expression of ZmCkx3, ZmCkx4, and ZmCkx5. The data demonstrate ZmCkx4 has a low constitutive expression in most organs with higher levels observed in ear, silk and vascular bundles, as well as intermediate levels in leaf and pedicels. ZmCkx3 expression was observed in roots. ZmCkx5 expression was highest in roots and vascular bundles.

FIG. 5 provides a schematic of various Mu insertions in ZmCkx2 and ZmCkx4.

FIG. 6 provides data as to number of shoots formed in transgenic Ubi:ZmCkx2 and control maize calli during the regeneration process.

FIG. 7 provides data as to phenotypic characteristics of transgenic Ubi:ZmCkx2 and control maize plants.

FIG. 8A shows the level of cytokinin oxidase activity in roots produced by calli expressing Ubi-ZmCkx2 compared to roots produced by control calli.

FIG. 8B shows the level of cytokinin oxidase activity in leaves of transgenic plants expressing Ubi-ZmCkx2 compared to transgenic controls.

FIG. 9 provides the PFAM alignment for ZmCkx2 (amino acids 63 to 220 of SEQ ID NO: 3), ZmCkx3 (amino acids 68 to 229 of SEQ ID NO: 6), ZmCkx4 (amino acids 44 to 213 of SEQ ID NO:10), and ZmCkx5 (amino acids 59 to 224 of SEQ ID NO:14). The PFAM consensus sequence is provided in SEQ ID NO:56.

### DETAILED DESCRIPTION OF THE INVENTION

The present invention now will be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the invention set forth herein will come to mind to one skilled in the art to which these inventions pertain, having the benefit of the teachings presented in the foregoing descriptions and the associated drawings. Therefore, it is to be understood that the invention is not to be limited to the specific embodiments disclosed, and that modifications and other embodiments are intended to be included within the scope of the appended claims. Although specific terms are employed herein, they are used in a generic and descriptive sense only and not for purposes of limitation.

### Compositions

Compositions of the invention include cytokinin oxidase (CKX) polypeptides and polynucleotides that are involved in modulating plant development, morphology, and physiology. Compositions of the invention further include CKX promoters that are capable of regulating transcription. In particular, the present invention provides for isolated polynucleotides comprising nucleotide sequences encoding the amino acid sequence shown in SEQ ID NO: 3, 6, 9, 12, or 53. Further provided are polypeptides having an amino acid sequence encoded by a polynucleotide described herein, for example those set forth in SEQ ID NO:1, 2, 4, 5, 7, 8, 10, 11, 51, or 52. Additional compositions include the CKX promoter sequences set forth in SEQ ID NO: 13, 14, 15, and 16.

The cytokinin oxidase polypeptides of the invention share sequence identity with members of the cytokinin oxidase family of proteins. Changes in cytokinin oxidase activity alter the cytokinin concentration in tissues, and thus cytokinin oxidase enzymes are important in controlling local cytokinin-dependent processes. The cytokinin oxidase enzyme is a FAD-containing oxidoreductase that catalyzes the degradation of cytokinins bearing unsaturated isoprenoid side chains. The free bases, isopentenyl-adenine (iP) and zeatin (Z), and their respective ribosides, are exemplary substrates.

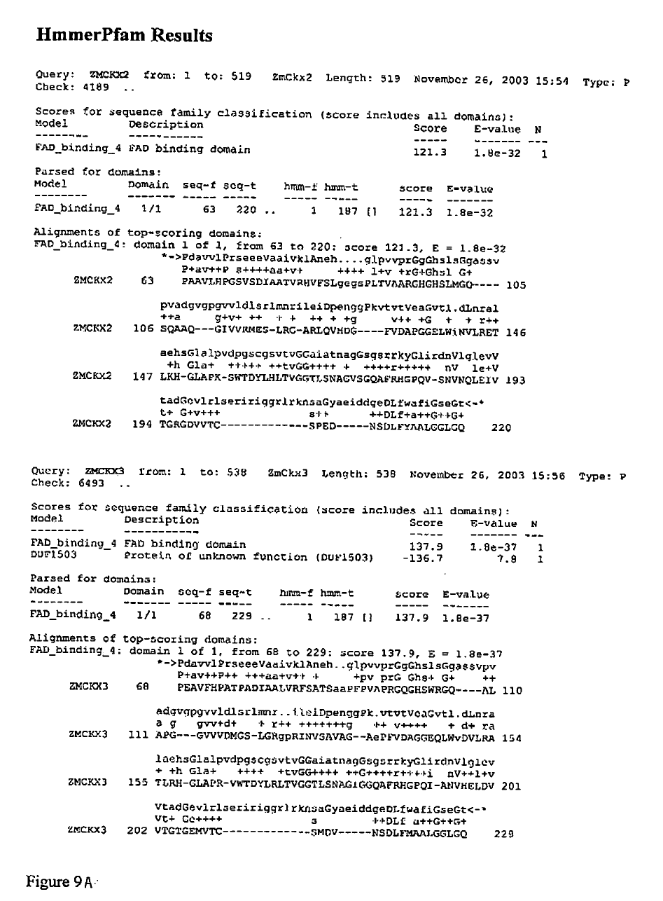

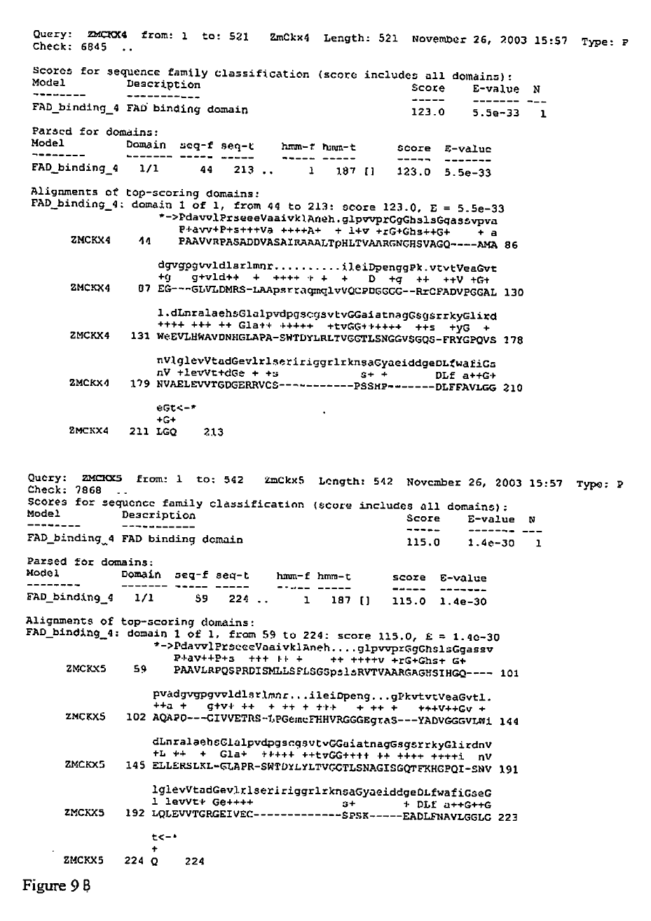

The CKX polypeptides of the invention contain a predicted FAD-binding domain (PFAM Accession No. PF01565). This family of enzymes comprises various polypeptides that use FAD as a co-factor. The FAD-binding domains are found from amino acid 63 to 220 of ZmCkx2, from amino acid 68 to 229 of ZmCkx3, from amino acid 44 to 213 of ZmCkx4 and from amino acid 59 to 224 of ZmCkx5. The alignments and the PFAM consensus sequence are provided in FIG. 9. The CKX polypeptides of the invention also share homology with several polypeptides in the CKX family. Table 1, appearing in Example 1 below, provides a summary of the sequence identity relationship of ZmCkx2, 3, 4, and 5 with various CKX family members.

The invention encompasses isolated or substantially purified polynucleotide or protein compositions. An “isolated” or “purified” polynucleotide or protein, or biologically active portion thereof, is substantially or essentially free from components that normally accompany or interact with the polynucleotide or protein as found in its naturally occurring environment. Thus, an isolated or purified polynucleotide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Fragments and variants of the disclosed polynucleotides and proteins encoded thereby are also encompassed by the present invention. By “fragment” is intended a portion of the polynucleotide or a portion of the amino acid sequence and hence of the protein encoded thereby. Fragments of a polynucleotide may encode protein fragments that retain the biological activity of the native protein and hence exhibit cytokinin oxidase activity. Alternatively, fragments of a polynucleotide that are useful as hybridization probes generally do not encode protein fragments retaining biological activity. Thus, fragments of a nucleotide sequence may range from at least about 20 nucleotides, about 50 nucleotides, about 100 nucleotides, and up to a full-length polynucleotide encoding a protein of the invention.

A fragment of a CKX polynucleotide that encodes a biologically active portion of a CKX protein of the invention will encode at least 15, 25, 30, 50, 100, 150, 200, 250, 300, 350, 400, 450, 500, 525, or 537 contiguous amino acids, or up to the total number of amino acids present in a full-length CKX protein of the invention (for example, 519 amino acids, 538 amino acids, 521 amino acids, and 542 amino acids for SEQ ID NO:3, 6, 9, and 12, respectively). Fragments of a CKX polynucleotide that are useful as hybridization probes or PCR primers generally need not encode a biologically active portion of a CKX protein.

Thus, a fragment of a CKX polynucleotide may encode a biologically active portion of a CKX protein, or it may be a fragment that can be used as a hybridization probe or PCR primer using methods disclosed below. A biologically active portion of a CKX protein can be prepared by isolating a portion of one of the CKX polynucleotides of the invention, expressing the encoded portion of the CKX protein (e.g., by recombinant expression in vitro), and assessing the activity of the encoded portion of the CKX protein. Polynucleotides that are fragments of a CKX nucleotide sequence comprise at least 16, 20, 50, 75, 100, 150, 200, 250, 300, 350, 400, 450, 500, 550, 600, 650, 700, 800, 900, 1000, 1100, 1200, 1300, 1400, 1500, 1600, or 1629 nucleotides, or up to the number of nucleotides present in a full-length CKX polynucleotide disclosed herein (for example, 3200 nucleotides, 1560 nucleotides, 3258 nucleotides, 2635 nucleotides, 1617 nucleotides, 6177 nucleotides, 1816 nucleotides, 1566 nucleotides, 5108 nucleotides or 1629 nucleotides for SEQ ID NO: 1, 2, 4, 5, 54, 7, 8, 55, 10, or 11, respectively).

“Variants” is intended to mean substantially similar sequences. For polynucleotides, a variant comprises a deletion and/or addition of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or amino acid sequence, respectively. For polynucleotides, conservative variants include those sequences that, because of the degeneracy of the genetic code, encode the amino acid sequence of one of the cytokinin oxidase polypeptides of the invention. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode a CKX protein of the invention. Generally, variants of a particular polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide of the invention (i.e., the reference polynucleotide) can also be evaluated by comparison of the percent sequence identity between the polypeptide encoded by a variant polynucleotide and the polypeptide encoded by the reference polynucleotide. Thus, for example, isolated polynucleotides that encode a polypeptide with a given percent sequence identity to the polypeptide of SEQ ID NO:3, 6, 9, 12, or 53 are disclosed. Percent sequence identity between any two polypeptides can be calculated using sequence alignment programs and parameters described elsewhere herein. Where any given pair of polynucleotides of the invention is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

“Variant” protein is intended to mean a protein derived from the native protein by deletion or addition of one or more amino acids at one or more sites in the native protein and/or substitution of one or more amino acids at one or more sites in the native protein. Variant proteins encompassed by the present invention are biologically active, that is they continue to possess the desired biological activity of the native protein, that is, cytokinin oxidase activity as described herein. Such variants may result from, for example, genetic polymorphism or from human manipulation. Biologically active variants of a native CKX protein of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the native protein as determined by sequence alignment programs and parameters described elsewhere herein. A biologically active variant of a protein of the invention may differ from that protein by as few as 1-15 amino acid residues, as few as 1-10, such as 6-10, as few as 5, as few as 4, 3, 2, or even 1 amino acid residue.

The proteins of the invention may be altered in various ways including amino acid substitutions, deletions, truncations, and insertions. Methods for such manipulations are generally known in the art. For example, amino acid sequence variants and fragments of the CKX proteins can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

Thus, the genes and polynucleotides of the invention include both the naturally occurring sequences as well as mutant forms. Likewise, the proteins of the invention encompass both naturally occurring proteins as well as variations and modified forms thereof. Such variants will continue to possess the desired cytokinin oxidase activity. Obviously, the mutations that will be made in the DNA encoding the variant must not place the sequence out of reading frame and optimally will not create complementary regions that could produce secondary mRNA structure. See, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by assaying for cytokinin oxidase activity.

Cytokinin oxidase activity can be assayed in a variety of ways. For example, a variety of cytokinin derivatives can be used as substrates to measure cytokinin oxidase activity. For instance, the polypeptide having CKX activity can be mixed with a cytokinin, for example, zeatin, and the net change of absorbance at 590 nm can be measured. See, U.S. Pat. No. 6,229,066. Alternatively, cytokinin oxidase activity can be measured by assaying for the conversion of [2-³H]iP to adenine. See, for example, Faiss et al. (1997) Plant J. 12:401-415, herein incorporated by reference. For additional assays, see Morris et al. (1999) Biochem Biophys Res Comm 255:328-333, Bilyeu et al. (2001) Plant Physiol 125: 378-386, Jones et al. (1990) Proceedings of the Plant Growth Regulation Society of America: (17^{th}), pp 183-196, Dietrich et al. (1995) Plant Physiol. Bioch. 268:327-336, and Frebort et al. (2002) Annu Biochem 306:1-7, each of which is herein incorporated by reference. In addition, a photospectrometric initial rate method which results in the formation of a formazan dye has been used to assay for cytokinin oxidase activity. See, for example, Frebort et al. (2002) Annu Biochem 306:1-7. In addition, cytokinin oxidase activity can be measured by assaying for a decrease in cytokinin levels in vivo. Such a decrease in cytokinin levels can produce one or more symptoms of a cytokinin-deficiency syndrome. The various phenotypes associated with cytokinin-deficiency syndrome are known in the art. See, for example, Schmulling et al. (2003) J. Plant Res 116: 241-252, herein incorporated by reference.

Variant polynucleotides and proteins also encompass sequences and proteins derived from a mutagenic and recombinogenic procedure such as DNA shuffling. With such a procedure, one or more different CKX sequences can be manipulated to create a new CKX polypeptide possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the CKX gene of the invention and other known CKX genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The compositions of the invention also include isolated polynucleotides comprising the CKX promoter nucleotide sequences set forth in SEQ ID NOS: 13, 14, 15, and 16. By “promoter” is intended a regulatory region of DNA usually comprising a TATA box capable of directing RNA polymerase II to initiate RNA synthesis at the appropriate transcription initiation site for a particular polynucleotide sequence. A promoter may additionally comprise other recognition sequences generally positioned upstream or 5′ to the TATA box, referred to as upstream promoter elements, which influence the transcription initiation rate. The promoter sequences of the present invention regulate (i.e., repress or activate) transcription from the promoter region.

It is recognized that additional domains can be added to the promoter sequences of the invention and thereby modulate the level of expression, the developmental timing of expression, or tissue type in which expression occurs. See particularly, Australian Patent No. AU-A-77751/94 and U.S. Pat. Nos. 5,466,785 and 5,635,618.

Fragments and variants of the disclosed CKX promoter polynucleotides are also encompassed by the present invention. Fragments of a promoter polynucleotide may retain biological activity and hence retain transcriptional regulatory activity. Alternatively, fragments of a polynucleotide that are useful as hybridization probes generally do not retain biological activity. Thus, fragments of a promoter nucleotide sequence may range from at least about 20 nucleotides, about 50 nucleotides, about 100 nucleotides, and up to the full-length polynucleotide of the invention.

Thus, a fragment of a CKX promoter polynucleotide may encode a biologically active portion of a CKX promoter, or it may be a fragment that can be used as a hybridization probe or PCR primer using methods disclosed below. A biologically active portion of a CKX promoter polynucleotide can be prepared by isolating a portion of one of the CKX promoter polynucleotides of the invention, and assessing the activity of the portion of the CKX promoter. Polynucleotides that are fragments of a CKX promoter polynucleotide comprise at least 16, 20, 50, 75, 100, 150, 200, 250, 300, 350, 400, 450, 500, 550, 600, 650, 700, 800, 900, 1,000, 1,100, 1,200, 1,300, 1,400, 1,500, 1,600, 1,700, 1,800, 1,900, or 2000 nucleotides, or up to the number of nucleotides present in a full-length CKX promoter polynucleotide disclosed herein (for example, 3003, 2001, 2448, or 2346 nucleotides for SEQ ID NO: 13, 14, 15, or 16, respectively).

For a promoter polynucleotide, a variant comprises a deletion and/or addition of one or more nucleotides at one or more sites within the native polynucleotide and/or a substitution of one or more nucleotides at one or more sites in the native polynucleotide. Generally, variants of a particular promoter polynucleotide of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variant promoter polynucleotides also encompass sequences derived from a mutagenic and recombinogenic procedure such as DNA shuffling. With such a procedure, one or more different promoter sequences can be manipulated to create a new CKX promoter possessing the desired properties. Strategies for such DNA shuffling are described elsewhere herein.

Methods are available in the art for determining if a promoter sequence retains the ability to regulate transcription. Such activity can be measured by Northern blot analysis. See, for example, Sambrook et al. (1989) Molecular Cloning: A Laboratory Manual (2d ed., Cold Spring Harbor Laboratory Press, Plainview, N.Y.), herein incorporated by reference. Alternatively, biological activity of the promoter can be measured using assays specifically designed for measuring the activity and/or level of the polypeptide being expressed from the promoter. Such assays are known in the art.

The polynucleotides of the invention (i.e., the CKX sequences and the CKX promoter sequences) can be used to isolate corresponding sequences from other organisms, particularly other plants, and more particularly other monocots. In this manner, methods such as PCR, hybridization, and the like can be used to identify such sequences based on their sequence homology to the sequences set forth herein. Sequences isolated based on their sequence identity to the entire CKX sequences or the CKX promoter sequences set forth herein or to variants and fragments thereof are encompassed by the present invention. Such sequences include sequences that are orthologs of the disclosed sequences. “Orthologs” is intended to mean genes derived from a common ancestral gene and which are found in different species as a result of speciation. Genes found in different species are considered orthologs when their nucleotide sequences and/or their encoded protein sequences share at least 60%, 70%, 75%, 80%, 85%, 90%, 91%, 92%, 93%, 94%, 95%, 96%, 97%, 98%, 99%, or greater sequence identity. Functions of orthologs are often highly conserved among species. Thus, isolated polynucleotides that encode for a CKX protein and which hybridize under stringent conditions to the CKX sequences disclosed herein, or to variants or fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known polynucleotide is used as a probe that selectively hybridizes to other corresponding polynucleotides present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as 32P, or any other detectable marker. Thus, for example, probes for hybridization can be made by labeling synthetic oligonucleotides based on the CKX polynucleotides or the CKX promoter sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, the entire CKX polynucleotide or the entire CKX promoter sequences disclosed herein, or one or more portions thereof, may be used as a probe capable of specifically hybridizing to corresponding CKX polynucleotides and messenger RNAs. To achieve specific hybridization under a variety of conditions, such probes include sequences that are unique among CKX polynucleotide sequences and are optimally at least about 10 nucleotides in length, and most optimally at least about 20 nucleotides in length. Such probes may be used to amplify corresponding CKX polynucleotides from a chosen plant by PCR. This technique may be used to isolate additional coding sequences from a desired plant or as a diagnostic assay to determine the presence of coding sequences in a plant. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, optimally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

Specificity is typically the function of post-hybridization washes, the critical factors being the ionic strength and temperature of the final wash solution. For DNA-DNA hybrids, the Tₘ can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is optimal to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Ilnterscience, New York). See Sambrook et al. (1989) Molecular Cloning: A Laboratory Manual (2d ed., Cold Spring Harbor Laboratory Press, Plainview, N.Y.).

The following terms are used to describe the sequence relationships between two or more polynucleotides or polypeptides: (a) “reference sequence”, (b) “comparison window”, (c) “sequence identity”, and, (d) “percentage of sequence identity.”

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two polynucleotides. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the GCG Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the GCG Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The invention further provides plants having altered levels and/or activities of the CKX polypeptides of the invention. In some embodiments, the plants of the invention have stably incorporated into their genomes the CKX sequences of the invention. In certain embodiments, plants that are genetically modified at a genomic locus encoding a CKX polypeptide of the invention are provided. By “native genomic locus” is intended a naturally occurring genomic sequence. In some embodiments, the genomic locus is set forth in SEQ ID NO:1, 4, 7, 10, or 51. In still further embodiments, the genomic locus is modified to reduce or eliminate the activity of the CKX polypeptide. The term “genetically modified” as used herein refers to a plant or plant part that is modified in its genetic information by the introduction of one or more foreign polynucleotides, and that the insertion of the foreign polynucleotide leads to a phenotypic change in the plant. By “phenotypic change” is intended a measurable change in one or more cell functions. For example, plants having the genetic modification at the genomic locus encoding the CKX polypeptide can show reduced or eliminated expression or activity of the CKX polypeptide. Various methods to generate such a genetically modified genomic locus are described elsewhere herein, as are the variety of phenotypes that can result from the modulation of the level and/or activity of the CKX sequences of the invention.

As used herein, the term plant includes plant cells, plant protoplasts, plant cell tissue cultures from which a plant can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, grain and the like. As used herein, by “grain” is intended the mature seed produced by commercial growers for purposes other than growing or reproducing the species. Progeny, variants, and mutants of the regenerated plants are also included within the scope of the invention, provided that these parts comprise the introduced nucleic acid sequences.

A “subject plant” or “subject plant cell” is one in which genetic alteration, such as transformation, has been effected as to a gene of interest, or is a plant or plant cell which is descended from a plant or plant cell so altered and which comprises the alteration. A “control” or “control plant” or “control plant cell” provides a reference point for measuring changes in the subject plant or plant cell.

A control plant or control plant cell may comprise, for example: (a) a wild-type plant or plant cell, i.e., of the same genotype as the starting material for the genetic alteration which resulted in the subject plant or subject plant cell; (b) a plant or plant cell of the same genotype as the starting material but which has been transformed with a null construct (i.e. with a construct which has no known effect on the trait of interest, such as a construct comprising a marker gene); (c) a plant or plant cell which is a non-transformed segregant among progeny of a subject plant or subject plant cell; (d) a plant or plant cell genetically identical to the subject plant or subject plant cell but which is not exposed to conditions or stimuli that would induce expression of the gene of interest; or (e) the subject plant or subject plant cell itself, under conditions in which the gene of interest is not expressed.

In the present case, for example, in various embodiments, changes in ctyokinin oxidase activity, cytokinin oxidase levels, cytokinin activity, cytokinin levels, cytokinin ratios, cytokinin distribution, and/or changes in one or more traits such as flowering time, seed set, branching, senescence, stress tolerance, or root mass, could be measured by comparing a subject plant or subject plant cell to a control plant or control plant cell.

### Methods

I. Providing Sequences

The sequences of the present invention can be introduced/expressed in a host cell such as bacteria, yeast, insect, mammalian, or optimally plant cells. It is expected that those of skill in the art are knowledgeable in the numerous systems available for the introduction of a polypeptide or a nucleotide sequence of the present invention into a host cell. No attempt to describe in detail the various methods known for providing proteins in prokaryotes or eukaryotes will be made.

By “host cell” is meant a cell which comprises a heterologous nucleic acid sequence of the invention. Host cells may be prokaryotic cells such as E. coli, or eukaryotic cells such as yeast, insect, amphibian, or mammalian cells. Host cells can also be monocotyledonous or dicotyledonous plant cells. In one embodiment, the monocotyledonous host cell is a maize host cell.

The use of the term “polynucleotide” is not intended to limit the present invention to polynucleotides comprising DNA. Those of ordinary skill in the art will recognize that polynucleotides can comprise ribonucleotides and combinations of ribonucleotides and deoxyribonucleotides. Such deoxyribonucleotides and ribonucleotides include both naturally occurring molecules and synthetic analogues. The polynucleotides of the invention also encompass all forms of sequences including, but not limited to, single-stranded forms, double-stranded forms, hairpins, stem-and-loop structures, and the like.

The CKX polynucleotide or CKX promoter sequences of the invention can be provided in expression cassettes for expression in the organism of interest. The cassette may include 5′ and 3′ regulatory sequences operably linked to a CKX polynucleotide of the invention. “Operably linked” is intended to mean a functional linkage between two or more elements. For example, an operable linkage between a polynucleotide of interest and a regulatory sequence (i.e., a promoter) is a functional link that allows for expression of the polynucleotide of interest. Operably linked elements may be contiguous or non-contiguous. When used to refer to the joining of two protein coding regions, by operably linked is intended that the coding regions are in the same reading frame. The cassette may additionally contain at least one additional gene to be cotransformed into the organism. Alternatively, any additional gene(s) can be provided on multiple expression cassettes. Such an expression cassette is provided with a plurality of restriction sites and/or recombination sites for insertion of the CKX polynucleotide to be under the transcriptional regulation of the regulatory regions. The expression cassette may additionally contain selectable marker genes.

The expression cassette may include in the 5′-3′ direction of transcription, a transcriptional and translational initiation region (i.e., a promoter), a CKX polynucleotide of the invention, and a transcriptional and translational termination region (i.e., termination region) functional in the host cell (i.e., the plant). The regulatory regions (i.e., promoters, transcriptional regulatory regions, and translational termination regions) and/or the CKX polynucleotide of the invention may be native/analogous to the host cell or to each other. Alternatively, the regulatory regions and/or the CKX polynucleotide of the invention may be heterologous to the host cell or to each other. As used herein, “heterologous” in reference to a sequence is a sequence that originates from a foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. As used herein, a chimeric gene comprises a coding sequence operably linked to a transcription initiation region that is heterologous to the coding sequence.

While heterologous promoters can be used to express the sequences, the native promoter sequences (i.e., SEQ ID NO: 13, 14, 15, or 16) also may be used. Such constructs can change expression levels of CKX in the plant or plant cell. Thus, the phenotype of the plant or plant cell can be altered. Alternatively, in other methods, any of the CKX promoter sequences of the invention can be used to express the CKX sequences. In addition, other CKX promoters can be used; see, for example, WO 02/0708438 and U.S. Publication 01525000; and U.S. patent applications Ser. Nos. 10/109,488 and 11/074,144 (SEQ ID NOS: 17 and 18 herein).

A termination region may be native with the transcriptional initiation region, may be native with the operably linked CKX polynucleotide of interest or with the CKX promoter sequences, may be native with the plant host, or may be derived from another source (i.e., foreign or heterologous) to the promoter, the CKX polynucleotide of interest, the plant host, or any combination thereof. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acids Res. 15:9627-9639.

Where appropriate, the polynucleotides may be optimized for increased expression in the transformed plant. That is, the polynucleotides can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Johnson et al. (1986) Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

The expression cassette can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D). Additional selectable markers include phenotypic markers such as β-galactosidase and fluorescent proteins such as green fluorescent protein (GFP) (Su et al. (2004) Biotechnol Bioeng 85:610-9 and Fetter et al. (2004) Plant Cell 16:215-28), cyan florescent protein (CYP) (Bolte et al. (2004) J. Cell Science 117:943-54 and Kato et al. (2002) Plant Physiol 129:913-42), and yellow florescent protein (PhiYFP™ from Evrogen, see, Bolte et al. (2004) J. Cell Science 117:943-54). For additional selectable markers, see generally, Yarranton (1992) Curr. Opin. Biotech. 3:506-511; Christopherson et al. (1992) Proc. Nat. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et a. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 ( Springer-Verlag, Berlin); Gill et al. (1988) Nature 334:721-724. Such disclosures are herein incorporated by reference. The above list of selectable marker genes is not meant to be limiting. Any selectable marker gene can be used in the present invention.

A number of promoters can be used in the practice of the invention, including the native promoter of the polynucleotide sequence of interest. The promoters can be selected based on the desired outcome. The nucleic acids can be combined with constitutive, tissue-preferred, or other promoters for expression in plants.

Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et a. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to target enhanced CKX expression within a particular plant tissue. Tissue-preferred promoters include those disclosed by Yamamoto et al. (1997) Plant J. 12(2):255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression. See, also, U.S. Patent Application No. 2003/0074698, herein incorporated by reference. Promoters active in maternal plant tissues, such as female florets, ovaries, aleurone, pedicel, and pedicel-forming region, either pre-pollination or upon pollination, may be of particular interest.

Leaf-preferred promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; Baszczynski et al. (1988) Nucl. Acid Res. 16:4732; Mitra et al. (1994) Plant Molecular Biology 26:35-93; Kayaya et al. (1995) Molecular and General Genetics 248:668-674; and Matsuoka et al. (1993) Proc. Nat. Acad. Sci. USA 90(20):9586-9590. Senescence regulated promoters are also of use, such as SAM22 (Crowell et al. (1992) Plant Mol. Biol. 18:459-466).

Root-preferred promoters are known and can be selected from the many available from the literature or isolated de novo from various compatible species. See, for example, Hire et al. (1992) Plant Mol. Biol. 20(2):207-218 (soybean root-specific glutamine synthetase gene); Keller and Baumgartner (1991) Plant Cell 3(10):1051-1061 (root-specific control element in the GRP 1.8 gene of French bean); Sanger et al. (1990) Plant Mol. Biol. 14(3):433-443 (root-specific promoter of the mannopine synthase (MAS) gene of Agrobacterium tumefaciens); and Miao et al. (1991) Plant Cell 3(1):11-22 (full-length cDNA clone encoding cytosolic glutamine synthetase (GS), which is expressed in roots and root nodules of soybean). See also Bogusz et al. (1990) Plant Cell 2(7):633-641, where two root-specific promoters isolated from hemoglobin genes from the nitrogen-fixing nonlegume Parasponia andersonii and the related non-nitrogen-fixing nonlegume Trema tomentosa are described. The promoters of these genes were linked to a β-glucuronidase reporter gene and introduced into both the nonlegume Nicotiana tabacum and the legume Lotus corniculatus, and in both instances root-specific promoter activity was preserved. Leach and Aoyagi (1991) describe their analysis of the promoters of the highly expressed roIC and roID root-inducing genes of Agrobacterium rhizogenes (see Plant Science (Limerick) 79(1):69-76). They concluded that enhancer and tissue-preferred DNA determinants are dissociated in those promoters. Teeri et al. (1989) used gene fusion to lacZ to show that the Agrobacterium T-DNA gene encoding octopine synthase is especially active in the epidermis of the root tip and that the TR2′ gene is root specific in the intact plant and stimulated by wounding in leaf tissue, an especially desirable combination of characteristics for use with an insecticidal or larvicidal gene (see EMBO J. 8(2):343-350). The TR1′ gene, fused to nptII (neomycin phosphotransferase II) showed similar characteristics. Additional root-preferred promoters include the VfENOD-GRP3 gene promoter (Kuster et al. (1995) Plant Mol. Biol. 29(4):759-772); rolB promoter (Capana et al. (1994) Plant Mol. Biol. 25(4):681-691; and the CRWAQ81 root-preferred promoter with the ADH first intron (U.S. patent application 10/961,629, filed Oct. 8, 2004, herein incorporated by reference). See also U.S. Pat. Nos. 5,837,876; 5,750,386; 5,633,363; 5,459,252; 5,401,836; 5,110,732; and 5,023,179. Promoters associated with the Ckx1 gene from maize may also be useful in modifying CKX activity in roots; see U.S. patent applications Ser. Nos. 10/109,488 and 11/074,144 (SEQ ID NOS: 17 and 18 herein).

“Seed-preferred” promoters include those promoters active during seed development, such as those expressed preferentially in female reproductive tissues, and those regulating seed storage proteins, as well as those promoters active during seed germination. See Thompson et al. (1989) BioEssays 10:108, herein incorporated by reference. Such seed-preferred promoters include, but are not limited to, maize zag2.1 promoter, (GenBank X80206); maize Zap promoter, also known as ZmMADS (U.S. patent publication 2004/0025206); maize eep1 promoter (U.S. patent publication 2004/0237147); maize lec1 promoter (U.S. patent application Ser. No. 09/718,754); maize F3.7 promoter (Baszczynski et al., Maydica (1997) 42:189-201 (1997); maize tb1 promoter (Hubbarda et al., Genetics (2002) 162:1927-1935); maize Zm40 promoter (U.S. Pat. No. 6,403,862 and WO 01/21783); maize mLIP15 promoter, U.S. Pat. No. 6,479,734; maize ESR promoter, U.S. patent publication 2004/0210960; maize PCNA 2 promoter (U.S. patent application 10/388,359 and WO 03/078591); Cim1 (cytokinin-induced message); cZ19B1 (maize 19 kDa zein); milps (myo-inositol-1-phosphate synthase) (see WO 00/11177 and U.S. Pat. No. 6,225,529; herein incorporated by reference). Gamma-zein is an endosperm-specific promoter. Globulin-1 (Glob-1) is a representative embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, gamma-zein, waxy, shrunken 1, shrunken 2, globulin 1, etc. See also WO 00/12733 and U.S. Pat. No. 6,528,704, where seed-preferred promoters from end1 and end2 genes are disclosed; herein incorporated by reference. Additional embryo specific promoters are disclosed in Sato et al. (1996) Proc. Natl. Acad. Sci. 93:8117-8122; Nakase et a. (1997) Plant J 12:235-46; and Postma-Haarsma et al. (1999) Plant Mol. Biol. 39:257-71. Additional endosperm specific promoters are disclosed in Albani et al. (1984) EMBO 3:1405-15; Albani et al. (1999) Theor. Appl. Gen. 98:1253-62; Albani et al. (1993) Plant J. 4:343-55; Mena et al. (1998) The Plant Journal 116:53-62, and Wu et al. (1998) Plant Cell Physiology 39:885-889.

Shoot-preferred promoters include shoot meristem-preferred promoters such as promoters disclosed in Weigal et al. (1992) Cell 69:843-859; Accession No. AJ131822; Accession No. Z71981; Accession No. AF049870 and shoot-preferred promoters disclosed in McAvoy et al. (2003) Acta Hort. (ISHS) 625:379-385.

Dividing cell or meristematic tissue-preferred promoters have been disclosed in Ito et al. (1994) Plant Mol. Biol. 24:863-878; Reyad et al. (1995) Mo. Gen. Genet. 248:703-711; Shaul et al. (1996) Proc. Natl. Acad. Sci. 93:4868-4872; Ito et al. (1997) Plant J. 11:983-992; and Trehin et al. (1997) Plant Mol. Biol. 35:667-672.

Inflorescence-preferred promoters include the promoter of chalcone synthase (Van der Meer et al. (1990) Plant Mol. Biol. 15:95-109), LAT52 (Twell et al. (1989) Mol. Gen. Genet. 217:240-245), pollen specific genes (Albani et al (1990) Plant Mol Biol. 15:605, Zm13 (Buerrero et al. (1993) Mol. Gen. Genet. 224:161-168), maize pollen-specific gene (Hamilton et al. (1992) Plant Mol. Biol. 18:211-218), sunflower pollen expressed gene (Baltz et al. (1992) The Plant Journal 2:713-721), B. napus pollen specific genes (Arnoldo et a. (1992) J. Cell. Biochem, Abstract No. Y101204).

Stress inducible promoters include salt/water stress-inducible promoters such as P5CS (Zang et al. (1997) Plant Sciences 129:81-89); cold-inducible promoters, such as cor15a (Hajela et al. (1990) Plant Physiol. 93:1246-1252), cor15b (Wlihelm et al. (1993) Plant Mol Biol 23:1073-1077), wsc120 (Ouellet et al. (1998) FEBS Lett. 423-324-328), ci7 (Kirch et al. (1997) Plant Mol Biol. 33:897-909), ci21A (Schneider et al. (1997) Plant Physiol. 113:335-45); drought-inducible promoters, such as, Trg-31 (Chaudhary et al (1996) Plant Mol. Biol. 30:1247-57), rd29 (Kasuga et al. (1999) Nature Biotechnology 18:287-291); osmotic inducible promoters, such as, Rab17 (Vilardell et al. (1991) Plant Mol. Biol. 17:985-93) and osmotin (Raghothama et al. (1993) Plant Mol Biol 23:1117-28); and heat inducible promoters, such as heat shock proteins (Barros et al. (1992) Plant Mol. 19:665-75; Marrs et al. (1993) Dev. Genet. 14:27-41), and smHSP (Waters et al. (1996) J. Experimental Botany 47:325-338). Other stress-inducible promoters include rip2 (U.S. Pat. No. 5,332,808 and U.S. Publication No. 2003/0217393) and rd29a (Yamaguchi-Shinozaki et al. (1993) Mol. Gen. Genetics 236:331-334).

The methods of the invention comprise introducing a polypeptide or polynucleotide into a host cell (i.e., a plant). “Introducing” is intended to mean presenting to the plant the polynucleotide or polypeptide in such a manner that the sequence gains access to the interior of a cell. The methods of the invention do not depend on a particular method for introducing a sequence into the host cell, only that the polynucleotide or polypeptides gains access to the interior of at least one cell of the host. Methods for introducing polynucleotide or polypeptides into host cells (i.e., plants) are known in the art and include, but are not limited to, stable transformation methods, transient transformation methods, and virus-mediated methods.

“Stable transformation” is intended to mean that the nucleotide construct introduced into a host (i.e., a plant) integrates into the genome of the plant and is capable of being inherited by the progeny thereof. “Transient transformation” is intended to mean that a polynucleotide is introduced into the host (i.e., a plant) and expressed temporally or a polypeptide is introduced into a host (i.e., a plant).

Transformation protocols as well as protocols for introducing polypeptides or polynucleotide sequences into plants may vary depending on the type of plant or plant cell, i.e., monocot or dicot, targeted for transformation. Suitable methods of introducing polypeptides and polynucleotides into plant cells include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

In specific embodiments, the CKX sequences of the invention can be provided to a plant using a variety of transient transformation methods. Such transient transformation methods include, but are not limited to, the introduction of the CKX protein or variants or fragments thereof directly into the plant, or the introduction of a CKX transcript into the plant. Such methods include, for example, microinjection or particle bombardment. See, for example, Crossway et al. (1986) Mol Gen. Genet. 202:179-185; Nomura et al. (1986) Plant Sci. 44:53-58; Hepler et al. (1994) Proc. Natl. Acad. Sci. 91: 2176-2180 and Hush et al. (1994) The Journal of Cell Science 107:775-784, all of which are herein incorporated by reference. Alternatively, the CKX polynucleotide can be transiently transformed into the plant using techniques known in the art. Such techniques include viral vector system and the precipitation of the polynucleotide in a manner that precludes subsequent release of the DNA. Thus, the transcription from the particle-bound DNA can occur, but the frequency with which it is released to become integrated into the genome is greatly reduced. Such methods include the use particles coated with polyethylimine (PEI; Sigma #P3143).

In certain embodiments, the polynucleotide of the invention may be introduced into plants by contacting plants with a virus or viral nucleic acids. Generally, such methods involve incorporating a nucleotide construct of the invention within a viral DNA or RNA molecule. It is recognized that the a CKX sequence of the invention may be initially synthesized as part of a viral polyprotein, which later may be processed by proteolysis in vivo or in vitro to produce the desired recombinant protein. Further, it is recognized that promoters of the invention also encompass promoters utilized for transcription by viral RNA polymerases. Methods for introducing polynucleotides into plants and expressing a protein encoded therein, involving viral DNA or RNA molecules, are known in the art. See, for example, U.S. Pat. Nos. 5,889,191, 5,889,190, 5,866,785, 5,589,367, 5,316,931, and Porta et al. (1996) Molecular Biotechnology 5:209-221; herein incorporated by reference.

Methods are known in the art for the targeted insertion of a polynucleotide at a specific location in the plant genome. In one embodiment, the insertion of the polynucleotide at a desired genomic location is achieved using a site-specific recombination system. See, for example, WO99/25821, WO99/25854, WO99/25840, WO99/25855, and WO99/25853; also U.S. Pat. Nos. 6,552,248, 6,624,297, 6,573,425, 6,455,315, and 6,458,594, all of which are herein incorporated by reference. Briefly, the polynucleotide of the invention can be contained in a transfer cassette flanked by two non-identical recombination sites. The transfer cassette is introduced into a plant having stably incorporated into its genome a target site which is flanked by two non-identical recombination sites that correspond to the sites of the transfer cassette. An appropriate recombinase is provided and the transfer cassette is integrated at the target site. The polynucleotide of interest is thereby integrated at a specific chromosomal position in the plant genome.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting progeny having appropriate expression of the desired phenotypic characteristic identified. Two or more generations may be grown to ensure that expression of the desired phenotypic characteristic is stably maintained and inherited, and then seeds harvested to ensure expression of the desired phenotypic characteristic has been achieved. In this manner, the present invention provides transformed seed (also referred to as “transgenic seed”) having a polynucleotide of the invention, for example, an expression cassette of the invention, stably incorporated into its genome.

Pedigree breeding starts with the crossing of two genotypes, such as an elite line of interest and one other line having one or more desirable characteristics (e.g., having stably incorporated a polynucleotide of the invention, having a modulated activity and/or level of the polypeptide of the invention, etc.) which complements the elite line of interest. If the two original parents do not provide all the desired characteristics, other sources can be included in the breeding population. In the pedigree method, superior plants are selfed and selected in successive filial generations. In the succeeding filial generations the heterozygous condition gives way to homogeneous lines as a result of self-pollination and selection. Typically in the pedigree method of breeding, five or more successive filial generations of selfing and selection are practiced: F1→F2; F2→F3; F3→F4; F4→F₅, etc. After a sufficient amount of inbreeding, successive filial generations will serve to increase seed of the developed inbred. Preferably, the inbred line comprises homozygous alleles at about 95% or more of its loci.

Backcrossing can be used to transfer one or more specifically desirable traits from one line, the donor parent, to an inbred called the recurrent parent, which has overall good agronomic characteristics yet lacks that desirable trait or traits. Backcrossing may be used in combination with pedigree breeding to modify an elite line of interest, and a hybrid is made using the modified elite line. However, the same procedure can be used to move the progeny toward the genotype of the recurrent parent but at the same time retain many components of the non-recurrent parent, by stopping the backcrossing at an early stage and proceeding with selfing and selection. For example, an F1, such as a commercial hybrid, is created. This commercial hybrid may be backcrossed to one of its parent lines to create a BC1 or BC2. Progeny are selfed and selected so that the newly developed inbred has many of the attributes of the recurrent parent and yet several of the desired attributes of the non-recurrent parent. This approach leverages the value and strengths of the recurrent parent for use in new hybrids and breeding.

Therefore, one embodiment of this invention is a method of making a backcross conversion of a maize inbred line of interest, comprising the steps of crossing a plant of the maize inbred line of interest with a donor plant comprising a mutant gene or transgene conferring a desired trait, selecting an F1 progeny plant comprising the mutant gene or transgene conferring the desired trait, and backcrossing the selected F1 progeny plant to a plant of the maize inbred line of interest. This method may further comprise the step of obtaining a molecular marker profile of the maize inbred line of interest and using the molecular marker profile to select for a progeny plant with the desired trait and the molecular marker profile of the inbred line of interest. In the same manner, this method may be used to produce F1 hybrid seed by adding a final step of crossing the desired trait-converted maize inbred line of interest with a different maize plant to make F1 hybrid maize seed comprising a mutant gene or transgene conferring the desired trait.

Recurrent selection is a method used in a plant breeding program to improve a population of plants. The method entails individual plants cross pollinating with each other to form progeny. The progeny are grown and the superior progeny selected by any number of selection methods, which include individual plant, half-sib progeny, full-sib progeny, selfed progeny and topcrossing. The selected progeny are cross-pollinated with each other to form progeny for another population. This population is planted and again superior plants are selected to cross pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar. A synthetic cultivar is the resultant progeny formed by the intercrossing of several selected inbreds.

Mass selection is a useful technique especially when used in conjunction with molecular marker enhanced selection. In mass selection seeds from individuals are selected based on phenotype and/or genotype. These selected seeds are then bulked and used to grow the next generation. Bulk selection requires growing a population of plants in a bulk plot, allowing the plants to self-pollinate, harvesting the seed in bulk and then using a sample of the seed harvested in bulk to plant the next generation. Instead of self pollination, directed pollination could be used as part of the breeding program.

Mutation breeding is one of many methods that could be used to introduce new traits into an elite line. Mutations that occur spontaneously or are artificially induced can be useful sources of variability for a plant breeder. The goal of artificial mutagenesis is to increase the rate of mutation for a desired characteristic. Mutation rates can be increased by many different means including temperature, long-term seed storage, tissue culture conditions, radiation (such as X-rays, Gamma rays (e.g. cobalt 60 or cesium 137), neutrons, (product of nuclear fission by uranium 235 in an atomic reactor), Beta radiation (emitted from radioisotopes such as phosphorus 32 or carbon 14), or ultraviolet radiation (preferably from 2500 to 2900 nm)), or chemical mutagens (such as base analogues (5-bromo-uracil), related compounds (8-ethoxy caffeine), antibiotics (streptonigrin), alkylating agents (sulfur mustards, nitrogen mustards, epoxides, ethylenamines, sulfates, sulfonates, sulfones, lactones), azide, hydroxylamine, nitrous acid, or acridines. Once a desired trait is observed through mutagenesis the trait may then be incorporated into existing germplasm by traditional breeding techniques, such as backcrossing. Details of mutation breeding can be found in Fehr's “Principles of Cultivar Development,” 1993, Macmillan Publishing Company, the disclosure of which is incorporated herein by reference. In addition, mutations created in other lines may be used to produce a backcross conversion of an elite line comprising such mutations.

The present invention may be used for transformation of any plant species, including, but not limited to, monocots and dicots. Examples of plant species of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia. integrifolia), almond (Prunus amygdalus), sugar beets (Beta vulgaris), sugarcane (Saccharum spp.), oats, barley, vegetables, ornamentals, grasses and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum.

In specific embodiments, plants of the present invention are crop plants (for example, corn (maize), alfalfa, sunflower, Brassica, soybean, cotton, safflower, peanut, sorghum, wheat, millet, tobacco, etc.). In certain embodiments, corn and soybean plants are optimal, and in yet other embodiments corn plants are optimal.

Other plants of interest include grain plants that provide seeds of interest, oil-seed plants, and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava bean, lentils, chickpea, etc.

Typically, an intermediate host cell will be used in the practice of this invention to increase the copy number of the cloning vector. With an increased copy number, the vector containing the nucleic acid of interest can be isolated in significant quantities for introduction into the desired plant cells. In one embodiment, plant promoters that do not cause expression of the polypeptide in bacteria are employed.

Prokaryotes most frequently are represented by various strains of E. coli; however, other microbial strains may also be used. Commonly used prokaryotic control sequences, which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived P L promoter and N-gene ribosome binding site (Shimatake et al. (1981) Nature 292:128). The inclusion of selection markers in DNA vectors transfected in E coli. is also useful. Examples of such markers include genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235); Mosbach et al. (1983) Nature 302:543-545).

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention.

Synthesis of heterologous polynucleotides in yeast is well known (Sherman et al. (1982) Methods in Yeast Genetics, Cold Spring Harbor Laboratory). Two widely utilized yeasts for production of eukaryotic proteins are Saccharomyces cerevisiae and Pichia pastors. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

A protein of the present invention, once expressed, can be isolated from yeast by lysing the cells and applying standard protein isolation techniques to the lysate. The monitoring of the purification process can be accomplished by using Western blot techniques or radioimmunoassay or other standard immunoassay techniques.

The sequences of the present invention can also be ligated to various expression vectors for use in transfecting cell cultures of, for instance, mammalian, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al. (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider (1987) J. Embryol. Exp. Morphol. 27:353-365).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague et al. (1983) J. Virol. 45:773-781). Additionally, gene sequences to control replication in the host cell may be incorporated into the vector such as those found in bovine papilloma virus type-vectors (Saveria-Campo (1985) DNA Cloning Vol. II a Practical Approach, D. M. Glover, Ed., IRL Press, Arlington, Va., pp. 213-238).

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art (Kuchler (1997) Biochemical Methods in Cell Culture and Virology, Dowden, Hutchinson and Ross, Inc.).

In certain embodiments, the polynucleotides of the present invention can be stacked with any combination of other polynucleotide sequences of interest in order to create a plant with a desired phenotype with respect to one or more traits. The combinations generated may include multiple copies of any one or more of the polynucleotides of interest.

These stacked combinations can be created by any method including, but not limited to, cross breeding plants by any conventional or TopCross methodology, or genetic transformation. If the traits are stacked by genetically transforming the plants, the polynucleotide sequences of interest can be combined at any time and in any order. For example, a transgenic plant comprising one or more desired traits can be used as the target to introduce further traits by subsequent transformation. The traits can be introduced simultaneously in a co-transformation protocol with the polynucleotides of interest provided by any combination of transformation cassettes. For example, if two sequences will be introduced, the two sequences can be contained in separate transformation cassettes (trans) or contained on the same transformation cassette (cis). Expression of the sequences can be driven by the same promoter or by different promoters. In certain cases, it may be desirable to introduce a transformation cassette that will suppress the expression of a polynucleotide of interest. This may be combined with any combination of other suppression cassettes or overexpression cassettes to generate the desired combination of traits in the plant.

II. Modulating the Concentration and/or Activity of a CKX Polypeptide

A method for modulating the concentration and/or activity of the polypeptide of the present invention in a plant is provided. In general, concentration and/or activity is increased or decreased by at least 1%, 5%, 10%, 20%, 30%, 40%, 50%, 60%, 70%, 80%, or 90% relative to a native control plant, plant part, or cell which did not have the sequence of the invention introduced. Modulation in the present invention may occur during and/or subsequent to growth of the plant to the desired stage of development. In specific embodiments, the polypeptides of the present invention are modulated in monocots, particularly maize.

A variety of methods can be employed to assay for a modulation in the concentration and/or activity of a CKX polypeptide. For instance, the expression level of the CKX polypeptide may be measured directly, for example, by assaying for the level of the CKX polypeptide in the plant (i.e., Western or Northern blot), or indirectly, for example, by assaying the cytokinin oxidase activity of the CKX polypeptide in the plant. Methods for measuring the cytokinin oxidase activity are described elsewhere herein. In specific embodiments, modulation of CKX polypeptide concentration and/or activity comprises the modulation (i.e., an increase or a decrease) in the level of cytokinin in the plant. Methods to measure the level and/or activity of cytokinin are known in the art and are discussed elsewhere herein. In still other embodiments, the level and/or activity of the CKX polypeptide is modulated in vegetative tissue, in reproductive tissue, or in both vegetative and reproductive tissue.

In one embodiment, the activity and/or concentration of the CKX polypeptide is modulated by introducing the polypeptide or the polynucleotide of the invention into the plant. Subsequently, a plant having the introduced sequence of the invention is selected using methods known to those of skill in the art such as, but not limited to, Southern blot analysis, DNA sequencing, PCR analysis, or phenotypic analysis. A plant or plant part altered or modified by the foregoing embodiments is grown under plant forming conditions for a time sufficient to modulate the concentration and/or activity of the CKX polypeptide in the plant. Plant forming conditions are well known in the art and discussed briefly elsewhere herein.

It is also recognized that the level and/or activity of the polypeptide may be modulated by employing a polynucleotide that is not capable of directing, in a transformed plant, the expression of a protein or an RNA. For example, the polynucleotides of the invention may be used to design polynucleotide constructs that can be employed in methods for altering or mutating a genomic nucleotide sequence in an organism. Such polynucleotide constructs include, but are not limited to, RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such nucleotide constructs and methods of use are known in the art. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference. It is therefore recognized that methods of the present invention do not depend on the incorporation of the entire polynucleotide into the genome, only that the plant or cell thereof is altered as a result of the introduction of the polynucleotide into a cell. In one embodiment of the invention, the genome may be altered following the introduction of the polynucleotide into a cell. For example, the polynucleotide, or any part thereof, may be incorporated into the genome of the plant. Alterations to the genome of the present invention include, but are not limited to, additions, deletions, and substitutions of nucleotides into the genome. While the methods of the present invention do not depend on additions, deletions, and substitutions of any particular number of nucleotides, it is recognized that such additions, deletions, or substitutions comprise at least one nucleotide.

Genetic constructs providing reduced expression of cytokinin oxidase genes may be used in combination with constructs providing futher modulation of effective levels of cytokinin in a plant, including increased biosynthesis of cytokinins, as described in co-pending U.S. application Ser. No. 09/545,334 filed Apr. 16, 1999, and U.S. patent publication No. 2004/0237147, published Nov. 24, 2004, herein incorporated by reference.

A Increasing the Activity and/or Level of a CKX Polypeptide

Methods are provided to increase the activity and/or level of a CKX polypeptide of the invention in a plant. Such increase in the level and/or activity of a CKX polypeptide of the invention can be achieved by providing to the plant a CKX polypeptide. The CKX polypeptide can be provided by introducing the amino acid sequence encoding the CKX polypeptide into the plant, introducing into the plant a nucleotide sequence encoding a CKX polypeptide, or by modifying a genomic locus encoding the CKX polypeptide of the invention.

As discussed elsewhere herein, many methods are known in the art for providing a polypeptide to a plant including, but not limited to, direct introduction of the polypeptide into the plant, and introducing into the plant (transiently or stably) a polynucleotide construct encoding a polypeptide having cytokinin oxidase activity. It is also recognized that the methods of the invention may employ a polynucleotide that is not capable of directing, in the transformed plant, the expression of a protein or an RNA. Thus, the level and/or activity of a CKX polypeptide may be increased by altering the gene encoding the CKX polypeptide or by altering or affecting its promoter. See, e.g., Kmiec, U.S. Pat. No. 5,565,350; Zarling et al., PCT/US93/03868. Therefore mutagenized plants that carry mutations in CKX genes, where the mutations increase expression of the CKX gene or increase the cytokinin oxidase activity of the encoded CKX polypeptide, are provided.

B. Reducing the Activity and/or Level of a CKX Polypeptide

Methods are provided to reduce or eliminate the activity of a CKX polypeptide of the invention by transforming a plant cell with an expression cassette that expresses a polynucleotide that inhibits the expression of the CKX polypeptide. The polynucleotide may inhibit the expression of the CKX polypeptide directly, by preventing translation of the CKX messenger RNA, or indirectly, by encoding a polypeptide that inhibits the transcription or translation of a CKX gene encoding a CKX polypeptide. Methods for inhibiting or eliminating the expression of a gene in a plant are well known in the art, and any such method may be used in the present invention to inhibit the expression of a CKX polypeptide.

In accordance with the present invention, the expression of a CKX polypeptide is inhibited if the protein level of the CKX polypeptide is less than the protein level of the same CKX polypeptide in a plant or plant part that has not been genetically modified or mutagenized to inhibit the expression of that CKX polypeptide. In particular embodiments of the invention, the protein level of the CKX polypeptide in a modified plant or plant part according to the invention is less than 70%, less than 60%, less than 50%, less than 40%, less than 30%, less than 20%, less than 10%, less than 5%, or less than 2% of the protein level of the same CKX polypeptide in a plant or plant part that is not a mutant or that has not been genetically modified to inhibit the expression of that CKX polypeptide. The expression level of the CKX polypeptide may be measured directly, for example, by assaying for the level of CKX polypeptide expressed in the plant cell or plant, or indirectly, for example, by measuring the cytokinin oxidase activity of the CKX polypeptide in the plant cell or plant, or by measuring the cytokinin level or activity in the plant or plant cell. Methods for performing such assays are described elsewhere herein.

In certain embodiments of the invention, the activity of the CKX polypeptides is reduced or eliminated by transforming a plant cell with an expression cassette comprising a polynucleotide encoding a polypeptide that inhibits the activity of a CKX polypeptide. The cytokinin oxidase activity of a CKX polypeptide is inhibited according to the present invention if the cytokinin oxidase activity of the CKX polypeptide is less than the cytokinin oxidase activity of the same CKX polypeptide in a plant that has not been modified to inhibit the cytokinin oxidase activity of that CKX polypeptide. In particular embodiments of the invention, the cytokinin oxidase activity of the CKX polypeptide in a modified plant according to the invention is less than 70%, less than 60%, less than 50%, less than 40%, less than 30%, less than 20%, less than 10%, or less than 5% of the cytokinin oxidase activity of the same CKX polypeptide in a plant that that has not been modified to inhibit the expression of that CKX polypeptide. The cytokinin oxidase activity of a CKX polypeptide is “eliminated” according to the invention when it is not detectable by the assay methods described elsewhere herein. Methods of determining the cytokinin oxidase activity of a CKX polypeptide are described elsewhere herein.

In other embodiments, the activity of a CKX polypeptide may be reduced or eliminated by disrupting the gene encoding the CKX polypeptide. The invention encompasses mutagenized plants that carry mutations in CKX genes, where the mutations reduce expression of the CKX gene or inhibit the cytokinin oxidase activity of the encoded CKX polypeptide.

Thus, many methods may be used to reduce or eliminate the activity of a CKX polypeptide. In addition, more than one method may be used to reduce the activity of a single CKX polypeptide. Non-limiting examples of methods of reducing or eliminating the expression of a CKX polypeptides are given below.

1. Polynucleotide-Based Methods:

In some embodiments of the present invention, a plant is transformed with an expression cassette that is capable of expressing a polynucleotide that inhibits the expression of a CKX polypeptide of the invention. The term “expression” as used herein refers to the biosynthesis of a gene product, including the transcription and/or translation of said gene product. For example, for the purposes of the present invention, an expression cassette capable of expressing a polynucleotide that inhibits the expression of at least one CKX polypeptide is an expression cassette capable of producing an RNA molecule that inhibits the transcription and/or translation of at least one CKX polypeptide of the invention. The “expression” or “production” of a protein or polypeptide from a DNA molecule refers to the transcription and translation of the coding sequence to produce the protein or polypeptide, while the “expression” or “production” of a protein or polypeptide from an RNA molecule refers to the translation of the RNA coding sequence to produce the protein or polypeptide.

Examples of polynucleotides that inhibit the expression of a CKX polypeptide are given below.

i. Sense Suppression/Cosuppression

In some embodiments of the invention, inhibition of the expression of a CKX polypeptide may be obtained by sense suppression or cosuppression. For cosuppression, an expression cassette is designed to express an RNA molecule corresponding to all or part of a messenger RNA encoding a CKX polypeptide in the “sense” orientation. Overexpression of this RNA molecule can result in reduced expression of the native gene. Accordingly, multiple plant lines transformed with the cosuppression expression cassette are screened to identify those that show the greatest inhibition of CKX polypeptide expression.

The polynucleotide used for cosuppression may correspond to all or part of the sequence encoding the CKX polypeptide, all or part of the 5′ and/or 3′ untranslated region of a CKX polypeptide transcript, or all or part of both the coding sequence and the untranslated regions of a transcript encoding a CKX polypeptide. In some embodiments where the polynucleotide comprises all or part of the coding region for the CKX polypeptide, the expression cassette is designed to eliminate the start codon of the polynucleotide so that no protein product will be translated.

Cosuppression may be used to inhibit the expression of plant genes to produce plants having undetectable protein levels for the proteins encoded by these genes. See, for example, Broin et al. (2002) Plant Cell 14:1417-1432. Cosuppression may also be used to inhibit the expression of multiple proteins in the same plant. See, for example, U.S. Pat. No. 5,942,657. Methods for using cosuppression to inhibit the expression of endogenous genes in plants are described in Flavell et al. (1994) Proc. Natl. Acad. Sci. USA 91:3490-3496; Jorgensen et al. (1996) Plant Mol. Biol. 31:957-973; Johansen and Carrington (2001) Plant Physiol. 126:930-938; Broin et al. (2002) Plant Cell 14:1417-1432; Stoutjesdijk et al. (2002) Plant Physiol. 129:1723-1731; Yu et al. (2003) Phytochemistry 63:753-763; and U.S. Pat. Nos. 5,034,323, 5,283,184, and 5,942,657; each of which is herein incorporated by reference. The efficiency of cosuppression may be increased by including a poly-dT region in the expression cassette at a position 3′ to the sense sequence and 5′ of the polyadenylation signal. See, U.S. patent Publication No. 20020048814, herein incorporated by reference. Typically, such a nucleotide sequence has substantial sequence identity to the sequence of the transcript of the endogenous gene, optimally greater than about 65% sequence identity, more optimally greater than about 85% sequence identity, most optimally greater than about 95% sequence identity. See, U.S. Pat. Nos. 5,283,184 and 5,034,323; herein incorporated by reference.

ii. Antisense Suppression

In some embodiments of the invention, inhibition of the expression of the CKX polypeptide may be obtained by antisense suppression. For antisense suppression, the expression cassette is designed to express an RNA molecule complementary to all or part of a messenger RNA encoding the CKX polypeptide. Overexpression of the antisense RNA molecule can result in reduced expression of the native gene. Accordingly, multiple plant lines transformed with the antisense suppression expression cassette are screened to identify those that show the greatest inhibition of CKX polypeptide expression.

The polynucleotide for use in antisense suppression may correspond to all or part of the complement of the sequence encoding the CKX polypeptide, all or part of the complement of the 5′ and/or 3′ untranslated region of the CKX transcript, or all or part of the complement of both the coding sequence and the untranslated regions of a transcript encoding the CKX polypeptide. In addition, the antisense polynucleotide may be fully complementary (i.e., 100% identical to the complement of the target sequence) or partially complementary (i.e., less than 100% identical to the complement of the target sequence) to the target sequence. Antisense suppression may be used to inhibit the expression of multiple proteins in the same plant. See, for example, U.S. Pat. No. 5,942,657. Furthermore, portions of the antisense nucleotides may be used to disrupt the expression of the target gene. Generally, sequences of at least 50 nucleotides, 100 nucleotides, 200 nucleotides, 300, 400, 450, 500, 550, or greater may be used. Methods for using antisense suppression to inhibit the expression of endogenous genes in plants are described, for example, in Liu et al. (2002) Plant Physiol. 129:1732-1743 and U.S. Pat. Nos. 5,759,829 and 5,942,657, each of which is herein incorporated by reference. Efficiency of antisense suppression may be increased by including a poly-dT region in the expression cassette at a position 3′ to the antisense sequence and 5′ of the polyadenylation signal. See, U.S. patent Publication No. 20020048814, herein incorporated by reference.

iii. Double-Stranded RNA Interference

In some embodiments of the invention, inhibition of the expression of a CKX polypeptide may be obtained by double-stranded RNA (dsRNA) interference. For dsRNA interference, a sense RNA molecule like that described above for cosuppression and an antisense RNA molecule that is fully or partially complementary to the sense RNA molecule are expressed in the same cell, resulting in inhibition of the expression of the corresponding endogenous messenger RNA.

Expression of the sense and antisense molecules can be accomplished by designing the expression cassette to comprise both a sense sequence and an antisense sequence. Alternatively, separate expression cassettes may be used for the sense and antisense sequences. Multiple plant lines transformed with the dsRNA interference expression cassette or expression cassettes are then screened to identify plant lines that show the greatest inhibition of CKX polypeptide expression. Methods for using dsRNA interference to inhibit the expression of endogenous plant genes are described in Waterhouse et al. (1998) Proc. Natl. Acad. Sci. USA 95:13959-13964, Liu et al. (2002) Plant Physiol. 129:1732-1743, and WO 99/49029, WO 99/53050, WO 99/61631, and WO 00/49035; each of which is herein incorporated by reference.

iv. Hairpin RNA Interference and Intron-Containing Hairpin RNA Interference

In some embodiments of the invention, inhibition of the expression of one or more CKX polypeptides may be obtained by hairpin RNA (hpRNA) interference or intron-containing hairpin RNA (ihpRNA) interference. These methods are highly efficient at inhibiting the expression of endogenous genes. See, Waterhouse and Helliwell (2003) Nat. Rev. Genet. 4:29-38 and the references cited therein.

For hpRNA interference, the expression cassette is designed to express an RNA molecule that hybridizes with itself to form a hairpin structure that comprises a single-stranded loop region and a base-paired stem. The base-paired stem region comprises a sense sequence corresponding to all or part of the endogenous messenger RNA encoding the gene whose expression is to be inhibited, and an antisense sequence that is fully or partially complementary to the sense sequence. Thus, the base-paired stem region of the molecule generally determines the specificity of the RNA interference. Alternatively, the base-paired stem region may comprise complementary sequences corresponding to a selected promoter region, resulting in silencing of a coding sequence operably linked to said selected promoter. See, for example, Mette et al. (2000) EMBO J 19(19):5194-5201. hpRNA molecules are highly efficient at inhibiting the expression of endogenous genes, and the RNA interference they induce is inherited by subsequent generations of plants. See, for example, Chuang and Meyerowitz (2000) Proc. Natl. Acad. Sci. USA 97:4985-4990; Stoutjesdijk et al. (2002) Plant Physiol. 129:1723-1731; and Waterhouse and Helliwell (2003) Nat. Rev. Genet. 4:29-38. Methods for using hpRNA interference to inhibit or silence the expression of genes are described, for example, in Chuang and Meyerowitz (2000) Proc. Natl. Acad. Sci. USA 97:4985-4990; Stoutjesdijk et al. (2002) Plant Physiol. 129:1723-1731; Waterhouse and Helliwell (2003) Nat. Rev. Genet. 4:29-38; Pandolfini et al. BMC Biotechnology 3:7, and U.S. patent Publication No. 20030175965; each of which is herein incorporated by reference. A transient assay for the efficiency of hpRNA constructs to silence gene expression in vivo has been described by Panstruga et al. (2003) Mol. Biol. Rep. 30:135-140, herein incorporated by reference.

For ihpRNA, the interfering molecules have the same general structure as for hpRNA, but the RNA molecule additionally comprises an intron that is capable of being spliced in the cell in which the ihpRNA is expressed. The use of an intron minimizes the size of the loop in the hairpin RNA molecule following splicing, and this increases the efficiency of interference. See, for example, Smith et al. (2000) Nature 407:319-320. In fact, Smith et al. show 100% suppression of endogenous gene expression using ihpRNA-mediated interference. Methods for using ihpRNA interference to inhibit the expression of endogenous plant genes are described, for example, in. Smith et al. (2000) Nature 407:319-320; Wesley et al. (2001) Plant J. 27:581-590; Wang and Waterhouse (2001) Curr. Opin. Plant Biol. 5:146-150; Waterhouse and Helliwell (2003) Nat. Rev. Genet. 4:29-38; Helliwell and Waterhouse (2003) Methods 30:289-295, and U.S. patent Publication No. 20030180945, each of which is herein incorporated by reference.

The expression cassette for hpRNA interference may also be designed such that the sense sequence and the antisense sequence do not correspond to an endogenous RNA. In this embodiment, the sense and antisense sequence flank a loop sequence that comprises a nucleotide sequence corresponding to all or part of the endogenous messenger RNA of the target gene. Thus, it is the loop region that determines the specificity of the RNA interference. See, for example, WO 02/00904, herein incorporated by reference.

v. Amplicon-Mediated Interference

Amplicon expression cassettes comprise a plant virus-derived sequence that contains all or part of the target gene but generally not all of the genes of the native virus. The viral sequences present in the transcription product of the expression cassette allow the transcription product to direct its own replication. The transcripts produced by the amplicon may be either sense or antisense relative to the target sequence (i.e., the messenger RNA for the CKX polypeptide). Methods of using amplicons to inhibit the expression of endogenous plant genes are described, for example, in Angell and Baulcombe (1997) EMBO J. 16:3675-3684, Angell and Baulcombe (1999) Plant J. 20:357-362, and U.S. Pat. No. 6,646,805, each of which is herein incorporated by reference.

vi. Ribozymes

In some embodiments, the polynucleotide expressed by the expression cassette of the invention is catalytic RNA or has ribozyme activity specific for the messenger RNA of the CKX polypeptide. Thus, the polynucleotide causes the degradation of the endogenous messenger RNA, resulting in reduced expression of the CKX polypeptide. This method is described, for example, in U.S. Pat. No. 4,987,071, herein incorporated by reference.

vii Small Interfering RNA or Micro RNA

In some embodiments of the invention, inhibition of the expression of a CKX polypeptide may be obtained by RNA interference by expression of a gene encoding a micro RNA (miRNA). miRNAs are regulatory agents consisting of about 22 ribonucleotides. miRNA are highly efficient at inhibiting the expression of endogenous genes. See, for example Javier et al. (2003) Nature 425: 257-263, herein incorporated by reference.

For miRNA interference, the expression cassette is designed to express an RNA molecule that is modeled on an endogenous miRNA gene. The miRNA gene encodes an RNA that forms a hairpin structure containing a 22-nucleotide sequence that is complementary to another endogenous gene (target sequence). For suppression of CKX expression, the 22-nucleotide sequence is selected from a CKX transcript sequence and contains 22 nucleotides of said CKX sequence in sense orientation and 21 nucleotides of a corresponding antisense sequence that is complementary to the sense sequence. miRNA molecules are highly efficient at inhibiting the expression of endogenous genes, and the RNA interference they induce is inherited by subsequent generations of plants.

2. Polypeptide-Based Inhibition of Gene Expression

In one embodiment, the polynucleotide encodes a zinc finger protein that binds to a gene encoding a CKX polypeptide, resulting in reduced expression of the gene. In particular embodiments, the zinc finger protein binds to a regulatory region of a CKX gene. In other embodiments, the zinc finger protein binds to a messenger RNA encoding a CKX polypeptide and prevents its translation. Methods of selecting sites for targeting by zinc finger proteins have been described, for example, in U.S. Pat. No. 6,453,242, and methods for using zinc finger proteins to inhibit the expression of genes in plants are described, for example, in U.S. patent Publication No. 20030037355; each of which is herein incorporated by reference.

3. Polypeptide-Based Inhibition of Protein Activity

In some embodiments of the invention, the polynucleotide encodes an antibody that binds to at least one CKX polypeptide, and reduces the cytokinin oxidase activity of the CKX polypeptide. In another embodiment, the binding of the antibody results in increased turnover of the antibody-CKX complex by cellular quality control mechanisms. The expression of antibodies in plant cells and the inhibition of molecular pathways by expression and binding of antibodies to proteins in plant cells are well known in the art. See, for example, Conrad and Sonnewald (2003) Nature Biotech. 21:35-36, incorporated herein by reference.

4. Gene Disruption

In some embodiments of the present invention, the activity of a CKX polypeptide is reduced or eliminated by disrupting the gene encoding the CKX polypeptide. The gene encoding the CKX polypeptide may be disrupted by any method known in the art. For example, in one embodiment, the gene is disrupted by transposon tagging. In another embodiment, the gene is disrupted by mutagenizing plants using random or targeted mutagenesis, and selecting for plants that have reduced cytokinin oxidase activity.

i. Transposon Tagging

In one embodiment of the invention, transposon tagging is used to reduce or eliminate the CKX activity of one or more CKX polypeptides. Transposon tagging comprises inserting a transposon within an endogenous CKX gene to reduce or eliminate expression of the CKX polypeptide. “CKX gene” is intended to mean the gene that encodes a CKX polypeptide according to the invention.

In this embodiment, the expression of one or more CKX polypeptide is reduced or eliminated by inserting a transposon within a regulatory region or coding region of the gene encoding the CKX polypeptide. A transposon that is within an exon, intron, 5′ or 3′ untranslated sequence, a promoter, or any other regulatory sequence of a CKX gene may be used to reduce or eliminate the expression and/or activity of the encoded CKX polypeptide.

Methods for the transposon tagging of specific genes in plants are well known in the art. See, for example, Maes et al. (1999) Trends Plant Sci. 4:90-96; Dharmapuri and Sonti (1999) FEMS Microbiol. Left 179:53-59; Meissner et al. (2000) Plant J. 22:265-274; Phogat et al. (2000) J. Biosci. 25:57-63; Walbot (2000) Curr. Opin. Plant Biol. 2:103-107; Gai et al. (2000) Nucleic Acids Res. 28:94-96; Fitzmaurice et al. (1999) Genetics 153:1919-1928). In addition, the TUSC process for selecting Mu insertions in selected genes has been described in Bensen et al. (1995) Plant Cell 7:75-84; Mena et al. (1996) Science 274:1537-1540; and U.S. Pat. No. 5,962,764; each of which is herein incorporated by reference.

ii. Mutant Plants with Reduced Activity

Additional methods for decreasing or eliminating the expression of endogenous genes in plants are also known in the art and can be similarly applied to the instant invention. These methods include other forms of mutagenesis, such as ethyl methanesulfonate-induced mutagenesis, deletion mutagenesis, and fast neutron deletion mutagenesis used in a reverse genetics sense (with PCR) to identify plant lines in which the endogenous gene has been deleted. For examples of these methods see Ohshima et al. (1998) Virology 243:472-481; Okubara et al. (1994) Genetics 137:867-874; and Quesada et al. (2000) Genetics 154:421-436; each of which is herein incorporated by reference. In addition, a fast and automatable method for screening for chemically induced mutations, TILLING (Targeting Induced Local Lesions In Genomes), using denaturing HPLC or selective endonuclease digestion of selected PCR products is also applicable to the instant invention. See McCallum et al. (2000) Nat. Biotechnol. 18:455-457, herein incorporated by reference.

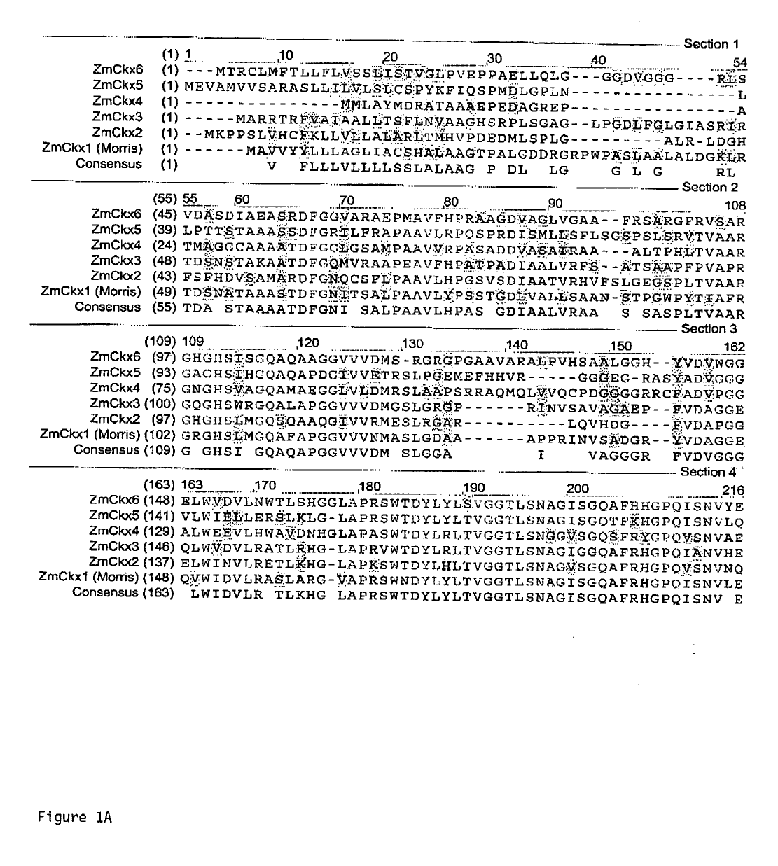

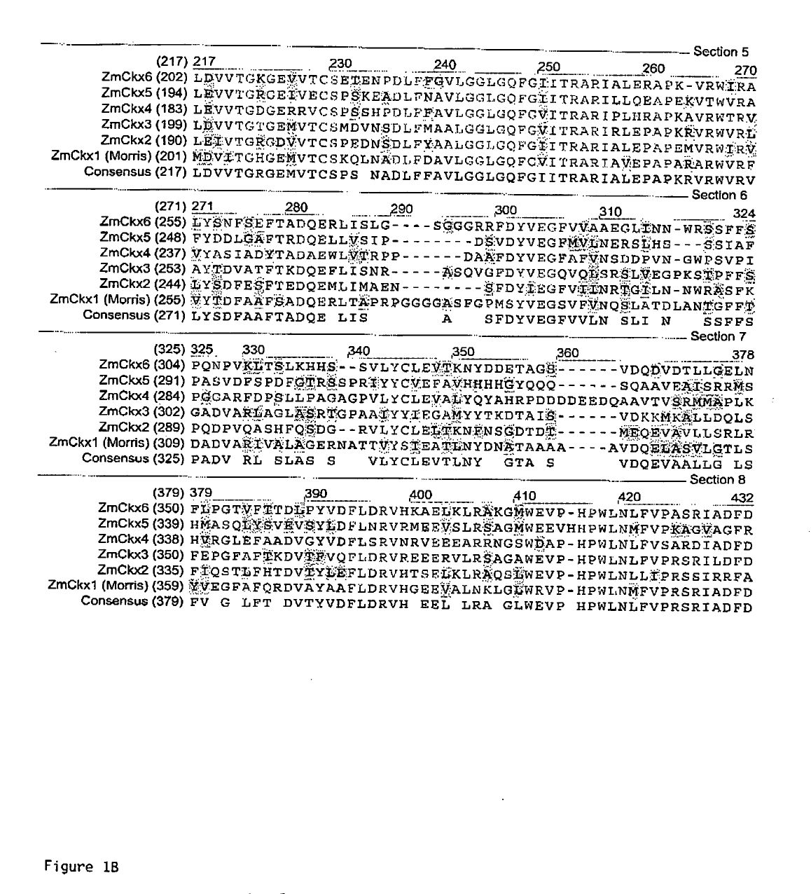

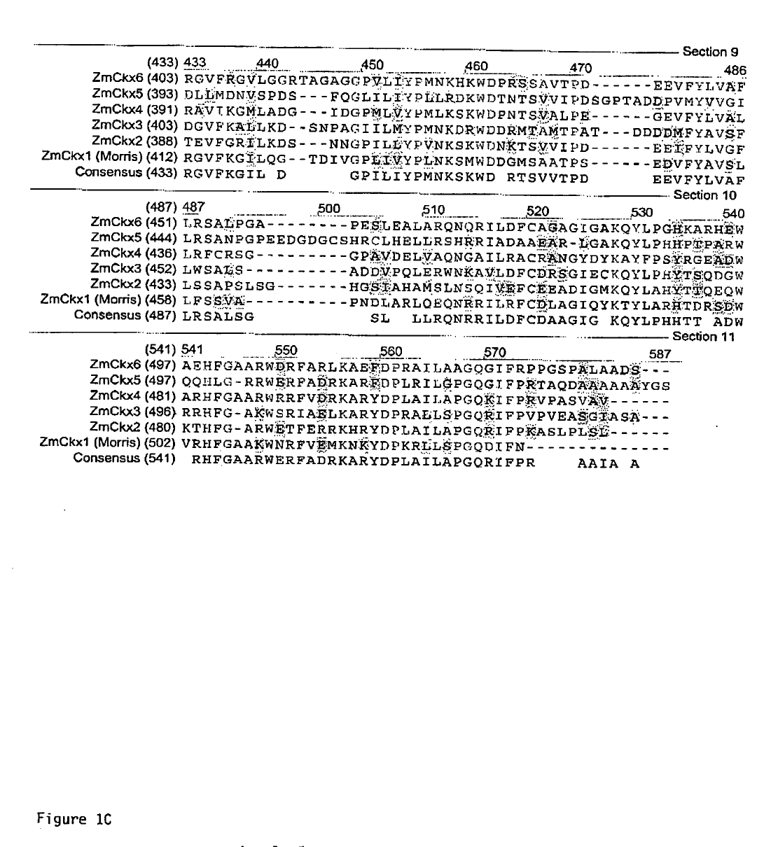

Mutations that impact gene expression or that interfere with the function (cytokinin oxidase activity) of the encoded protein are well known in the art. Insertional mutations in gene exons usually result in null-mutants. Mutations in conserved residues are particularly effective in inhibiting the cytokinin oxidase activity of the encoded protein. Conserved residues of plant CKX polypeptides suitable for mutagenesis with the goal to eliminate cytokinin oxidase activity have been described. See, for-example, FIGS. 1 and 9 and examples 1 and 7. Such mutants can be isolated according to well-known procedures, and mutations in different CKX loci can be stacked by genetic crossing. See, for example, Gruis et al. (2002) Plant Cell 14:2863-2882.

In another embodiment of this invention, dominant mutants can be used to trigger RNA silencing due to gene inversion and recombination of a duplicated gene locus. See, for example, Kusaba etal. (2003) Plant Cell 15:1455-1467.

The invention encompasses additional methods for reducing or eliminating the activity of one or more CKX polypeptides. Examples of other methods for altering or mutating a genomic nucleotide sequence in a plant are known in the art and include, but are not limited to, the use of RNA:DNA vectors, RNA:DNA mutational vectors, RNA:DNA repair vectors, mixed-duplex oligonucleotides, self-complementary RNA:DNA oligonucleotides, and recombinogenic oligonucleobases. Such vectors and methods of use are known in the art. See, for example, U.S. Pat. Nos. 5,565,350; 5,731,181; 5,756,325; 5,760,012; 5,795,972; and 5,871,984; each of which are herein incorporated by reference. See also, WO 98/49350, WO 99/07865, WO 99/25821, and Beetham et al. (1999) Proc. Natl. Acad. Sci. USA 96:8774-8778; each of which is herein incorporated by reference.

III. Modulating Cytokinin Level/Activity

As used herein a “cytokinin” refers to a class of plant-specific hormones that play a central role during the cell cycle and influence numerous developmental programs. Cytokinins comprise an N⁶-substituted purine derivative. Representative cytokinins include isopentenyladenine (N⁶-(Δ²-isopentenyl)adenine (hereinafter, iP), zeatin (6-(4-hydroxy-3methylbut-trans-2-enylamino)purine) (hereinafter, Z), and dihydrozeatin (DZ). The free bases and their ribosides (iPR, ZR, and DZR) are believed to be the active compounds. Additional cytokinins are known. See, for example, U.S. Pat. No. 5,211,738.

“Modulating the level and/or activity of cytokinin” includes any decrease or increase in cytokinin level and/or activity in the plant. For example, modulating the level and/or activity can comprise either an increase or a decrease in overall cytokinin content of about 0.1%, 0.5%, 1%, 3% 5%, 10%, 15%, 20%, 25%, 30%, 35%, 40%, 45%, 50%, 55%, 60%, 65%, 70%, 75%, 80%, 85%, 90%, 95%, or greater when compared to a control plant or plant part. Alternatively, the modulated level and/or activity of the cytokinin can include about a 0.5 fold, 1 fold, 2 fold, 4 fold, 8 fold, 16 fold, or 32 fold increase or decrease in cytokinin level/activity in the plant or a plant part when compared to a control plant or plant part.

It is further recognized that the modulation of the cytokinin level/activity need not be an overall increase/decrease in cytokinin level and/or activity, but also includes a change in tissue distribution of the cytokinin. For example, CKX polypeptides may influence the amount of cytokinin imported into specific tissues or exported from a cytokinin producing tissue. For example, import of cytokinin in sink tissues may involve an apoplastic transport step, where CKX polypeptides control the level of physiologically active cytokinins. See, for example, Jones et al. (1997) Plant Growth Regul 23:123-134, Turner et al. (1985) Plant Physiol 79:321-322, and Mok et al. (2001) Annu Rev Plant Physiol Plant Mol Biol 52:89-118, each of which are herein incorporated by reference.

Moreover, the modulation of the cytokinin level/activity need not be an overall increase/decrease in cytokinins, but also includes a change in the ratio of various cytokinin derivatives. For example, the ratio of various cytokinin derivatives such as isopentenyladenine-type, zeatin-type, or dihydrozeatin-type cytokinins, and the like, could be altered and thereby modulate the level/activity of the cytokinin of the plant or plant part when compared to a control plant.

Methods for assaying for a modulation in cytokinin level and/or activity are known in the art. For example, representative methods for cytokinin extraction, immunopurification, HPLC separation, and quantification by ELISA methods can be found in Faiss et al. (1997) Plant J. 12:401-415. See also Werner et al. (2001) PNAS 98:10487-10492) and Dewitte et al. (1999) Plant Physiol. 119:111-121. Each of these references is herein incorporated by reference.

In specific methods, the level and/or activity of a cytokinin in a plant is decreased by increasing the level or activity of the CKX polypeptide in the plant. Methods for increasing the level and/or activity of CKX polypeptides in a plant are discussed elsewhere herein. Briefly, such methods comprise providing a CKX polypeptide of the invention to a plant and thereby increasing the level and/or activity of the CKX polypeptide. In other embodiments, a CKX nucleotide sequence encoding a CKX polypeptide can be provided by introducing into the plant a polynucleotide comprising a CKX nucleotide sequence of the invention, expressing the CKX sequence, increasing the activity of the CKX polypeptide, and thereby decreasing the level and/or activity of a cytokinin in the plant or plant part. In certain embodiments, the CKX nucleotide construct introduced into the plant is stably incorporated into the genome of the plant.

In other methods, the level and/or activity of a cytokinin in a plant is increased by decreasing the level and/or activity of the CKX polypeptide in the plant. Such methods are disclosed in detail elsewhere herein. In one such method, a CKX nucleotide sequence is introduced into the plant and expression of said CKX nucleotide sequence decreases the activity of the CKX polypeptide, and thereby increasing the level and/or activity of a cytokinin in the plant or plant part. In certain embodiments, the CKX nucleotide construct introduced into the plant is stably incorporated into the genome of the plant.

As discussed above, one of skill will recognize the appropriate promoter to use to modulate the level/activity of a cytokinin in the plant. Exemplary promoters for this embodiment have been disclosed elsewhere herein.

Accordingly, the present invention further provides plants having a modulated level/activity of a cytokinin when compared to the cytokinin level/activity of a control plant. In one embodiment, the plant of the invention has an increased level/activity of the CKX polypeptide of the invention and thus has a decreased level/activity of cytokinin. In other embodiments, the plant of the invention has a reduced or eliminated level of the CKX polypeptide of the invention and thus has an increased level/activity of a cytokinin. In certain embodiments, such plants have stably incorporated into their genome a nucleic acid molecule comprising a CKX nucleotide sequence of the invention operably linked to a promoter that drives expression in the plant cell.

IV. Modulating Root Development

Methods for modulating root development in a plant are provided. By “modulating root development” is intended any alteration in the development of the plant root when compared to a control plant. Such alterations in root development include, but are not limited to, alterations in the growth rate of the primary root, the fresh root weight, the extent of lateral and adventitious root formation, the vasculature system, meristem development, or radial expansion.

Methods for modulating root development in a plant are provided. The methods comprise modulating the level and/or activity of the CKX polypeptide in the plant. In one method, a CKX sequence of the invention is provided to the plant. In another method, the CKX nucleotide sequence is provided by introducing into the plant a polynucleotide comprising a CKX nucleotide sequence of the invention, expressing the CKX sequence, and thereby modifying root development. In still other methods, the CKX nucleotide construct introduced into the plant is stably incorporated into the genome of the plant.

In other methods, root development is modulated by increasing the level or activity of the CKX polypeptide in the plant. An increase in CKX activity can result in one or more alterations to root development, including, but not limited to, larger root meristems, increased root growth, enhanced radial expansion, an enhanced vasculature system, increased root branching, more adventitious roots, and/or an increase in fresh root weight when compared to a control plant.

As used herein, “root growth” encompasses all aspects of growth of the different parts that make up the root system at different stages of its development in both monocotyledonous and dicotyledonous plants. It is to be understood that enhanced root growth can result from enhanced growth of one or more of its parts including the primary root, lateral roots, adventitious roots, etc.

Methods of measuring such developmental alterations in the root system are known in the art. See, for example, U.S. application No. 2003/0074698 and Werner et al. (2001) PNAS 18:10487-10492, both of which are herein incorporated by reference.

As discussed above, one of skill will recognize the appropriate promoter to use to modulate root development in the plant. Exemplary promoters for this embodiment include constitutive promoters and root-preferred promoters. Exemplary root-preferred promoters have been disclosed elsewhere herein.

Stimulating root growth and increasing root mass by increasing the activity and/or level of the CKX polypeptide also finds use in improving the standability of a plant. The term “resistance to lodging” or “standability” refers to the ability of a plant to fix itself to the soil. For plants with an erect or semi-erect growth habit, this term also refers to the ability to maintain an upright position under adverse conditions, such as adverse environments. This trait relates to the size, depth and morphology of the root system. In addition, stimulating root growth and increasing root mass by increasing the level and/or activity of the CKX polypeptide also finds use in promoting in vitro propagation of explants.

Furthermore, higher root biomass production due to an increased level and/or activity of CKX activity has a direct effect on the yield and an indirect effect on production of compounds produced by root cells or transgenic root cells or cell cultures of said transgenic root cells. One example of an interesting compound produced in root cultures is shikonin, the yield of which can be advantageously enhanced by said methods.

Accordingly, the present invention further provides plants having modulated root development when compared to the root development of a control plant. In some embodiments, the plant of the invention has an increased level/activity of the CKX polypeptide of the invention and has enhanced root growth and/or root biomass. In certain embodiments, such plants have stably incorporated into their genome a nucleic acid molecule comprising a CKX nucleotide sequence of the invention operably linked to a promoter that drives expression in the plant cell.

V. Modulating Shoot and Leaf Development

Methods are also provided for modulating shoot and leaf development in a plant. By “modulating shoot and/or leaf development” is intended any alteration in the development of the plant shoot and/or leaf. Such alterations in shoot and/or leaf development include, but are not limited to, alterations in shoot meristem development, in leaf number, leaf size, leaf and stem vasculature, internode length, and leaf senescence. As used herein, “leaf development” and “shoot development” encompasses all aspects of growth of the different parts that make up the leaf system and the shoot system, respectively, at different stages of their development, both in moriocotyledonous and dicotyledonous plants. Methods for measuring such developmental alterations in the shoot and leaf system are known in the art. See, for example, Werner et al. (2001) PNAS 98:10487-10492 and U.S. application No. 2003/0074698, each of which is herein incorporated by reference.

The method for modulating shoot and/or leaf development in a plant comprises modulating the activity and/or level of a CKX polypeptide of the invention. In one embodiment, a CKX sequence of the invention is provided. In other embodiments, the CKX nucleotide sequence can be provided by introducing into the plant a polynucleotide comprising a CKX nucleotide sequence of the invention, expressing the CKX sequence, and thereby modifying shoot and/or leaf development. In certain embodiments, the CKX nucleotide construct introduced into the plant is stably incorporated into the genome of the plant.

In specific embodiments, shoot or leaf development is modulated by increasing the level and/or activity of the CKX polypeptide in the plant. An increase in CKX activity can result in one or more alterations in shoot and/or leaf development, including, but not limited to, smaller apical meristems, reduced leaf number, reduced leaf surface, reduced vasculature, shorter internodes and stunted growth, and retarded leaf senescence, when compared to a control plant. Thus, the methods of the invention may find use in producing dwarf plants.

In certain embodiments, the level and/or activity of the CKX polypeptide in the plant is decreased to result in higher cytokinin levels. As discussed elsewhere herein, targeted reduction in CKX polypeptide level and/or activity may result in one or more of modulated floral development, modulated flowering time, increased seed size and/or increased seed weight, increased plant yield and/or plant vigor, improved or maintained stress tolerance, altered roovshoot ratio, or an increase in shoot growth, when compared to a control plant.

As discussed above, one of skill will recognize the appropriate promoter to use to modulate shoot and leaf development of the plant. Exemplary promoters for this embodiment include constitutive promoters, shoot-preferred promoters, shoot meristem-preferred promoters, and leaf-preferred promoters. Exemplary promoters have been disclosed elsewhere herein.

Accordingly, the present invention further provides plants having modulated shoot and/or leaf development when compared to a control plant. In some embodiments, the plant of the invention has an increased level/activity of the CKX polypeptide of the invention. In other embodiments, the plant of the invention has a decreased level/activity of the CKX polypeptide of the invention.

VI. Modulating Reproductive Tissue Development

Methods for modulating reproductive tissue development are provided. In one embodiment, methods are provided to modulate floral development in a plant. By “modulating floral development” is intended any alteration in a structure of a plant's reproductive tissue as compared to a control plant in which the activity or level of the CKX polypeptide has not been modulated. “Modulating floral development” further includes any alteration in the timing of the development of a plant's reproductive tissue (i.e., a delayed or a accelerated timing of floral development) when compared to a control plant in which the activity or level of the CKX polypeptide has not been modulated. Macroscopic alterations may include changes in size, shape, number, or location of reproductive organs, the developmental time period over which these structures form, and/or the ability to maintain or proceed through the flowering process in times of environmental stress. Microscopic alterations may include changes to the types or shapes of cells that make up the reproductive organs.

The method for modulating floral development in a plant comprises modulating CKX activity in a plant. In one method, a CKX sequence of the invention is provided. A CKX nucleotide sequence can be provided by introducing into the plant a polynucleotide comprising a CKX nucleotide sequence of the invention, expressing the CKX sequence, and thereby modifying floral development. In certain embodiments, the CKX nucleotide construct introduced into the plant is stably incorporated into the genome of the plant.

In specific methods, floral development is modulated by increasing the level or activity of the CKX polypeptide in the plant. An increase in CKX activity can result in one or more alterations in floral development, including, but not limited to, retarded flowering, reduced number of flowers, partial male sterility, and reduced seed set, when compared to a control plant. Inducing delayed flowering or inhibiting flowering can be used to enhance yield in forage crops such as alfalfa. Methods for measuring such developmental alterations in floral development are known in the art. See, for example, Mouradov et al. (2002) The Plant Cell S111-S130, herein incorporated by reference.

As discussed above, one of skill will recognize the appropriate promoter to use to modulate floral development of the plant. Exemplary promoters for this embodiment include constitutive promoters, inducible promoters, shoot-preferred promoters, and inflorescence-preferred promoters.

In other methods, floral development is modulated by decreasing the level and/or activity of the CKX sequence of the invention. Such methods can comprise introducing a CKX nucleotide sequence into the plant and decreasing the activity of the CKX polypeptide. In other methods, the CKX nucleotide construct introduced into the plant is stably incorporated into the genome of the plant. Decreasing expression of the CKX sequence of the invention can modulate floral development during periods of stress. Such methods are described elsewhere herein.

Accordingly, the present invention further provides plants having modulated floral development when compared to the floral development of a control plant. Compositions include plants having an increased level/activity of the CKX polypeptide of the invention and having an altered floral development. Compositions also include plants having a decreased level/activity of the CKX polypeptide of the invention wherein the plant maintains or proceeds through the flowering process in times of stress.

Methods are also provided for the use of the CKX sequences of the invention to increase seed size and/or weight. The method comprises decreasing the activity of the CKX sequences in a plant or plant part, such as the seed, by means of downregulation techniques described elsewhere herein. An increase in seed size and/or weight comprises an increased size or weight of the seed and/or an increase in the size or weight of one or more seed parts including, for example, the embryo, endosperm, seed coat, aleurone, or cotyledon.

As discussed above, one of skill will recognize an appropriate promoter to use to increase seed size and/or seed weight. Exemplary promoters of this embodiment include constitutive promoters, inducible promoters, seed-preferred promoters, embryo-preferred promoters, endosperm-preferred promoters, and promoters active in female reproductive tissues immediately pre- and post-pollination.

It is further recognized that increasing seed size and/or weight can also be accompanied by an increase in the speed of growth of seedlings or an increase in early vigor. As used herein, the term “early vigor” refers to the ability of a plant to grow rapidly during early development, and relates to the successful establishment, after germination, of a well-developed root system and a well-developed photosynthetic apparatus. In addition, an increase in seed size and/or weight can result in an increase in plant yield when compared to a control.

Accordingly, the present invention further provides plants having an increased seed weight and/or seed size when compared to a control plant. In other embodiments, plants having an increased vigor and plant yield are also provided. In some embodiments, the plant of the invention has a decreased level/activity of the CKX polypeptide of the invention and has an increased seed weight and/or seed size. In certain embodiments, such plants have stably incorporated into their genome a nucleic acid molecule comprising a CKX nucleotide sequence of the invention operably linked to a promoter that drives expression in the plant cell.

VII. Modulating the Stress Tolerance of a Plant

Methods are provided for the use of the CKX sequences of the invention to modify the tolerance of a plant to abiotic stress. Increases in the growth of seedlings or early vigor are often associated with increase in stress tolerance. For example, faster development of seedlings, including the root system of seedlings upon germination, is critical for survival, particularly under adverse conditions such as drought. Promoters that can be used in this method are described elsewhere herein. Briefly, constitutive promoters or root-preferred or stress-induced promoters could be used in this method. Accordingly, in one method of the invention, a plant's tolerance to stress is increased or maintained when compared to a control plant by decreasing the level of CKX activity in the plant. In other methods, a CKX nucleotide sequence is provided by introducing into the plant a polynucleotide comprising a CKX nucleotide sequence of the invention, expressing the CKX sequence, and thereby increasing the plant's tolerance to stress. In certain embodiments, the CKX nucleotide construct introduced into the plant is stably incorporated into the genome of the plant.

Methods are also provided to increase or maintain seed set during abiotic stress episodes. During periods of stress (i.e., drought, salt, heavy metals, temperature, etc.) embryo development is often aborted. In maize, halted embryo development results in aborted kernels on the ear. Preventing this kernel loss will maintain yield. Accordingly, methods are provided to increase the stress resistance in a plant (i.e., an early developing embryo). Decreasing expression of the CKX sequence of the invention can also modulate floral development during periods of stress, and thus methods are provided to maintain or improve the flowering process in plants under stress. The method comprises decreasing the level and/or activity of the CKX sequence of the invention by means of downregulation techniques described elsewhere herein.

Significant yield instability can occur as a result of unfavorable environments, especially during the lag phase of seed development. During this period, seeds undergo dramatic changes in ultra structure, biochemistry, and sensitivity to environmental perturbations yet demonstrate little change in dry mass accumulation. Two important events that occur during the lag phase are initiation and division of endosperm cells and amyloplasts (which are the sites for starch deposition). It has been demonstrated that during the lag phase (in maize, from pollination to about 10 to 12 days after pollination (DAP)), a dramatic increase in cytokinin concentration immediately precedes maximum rates of endosperm cell division and amyloplast formation, indicating that this hormone plays a central role in these processes and in what is called the ‘sink strength’ of the developing seed. Cytokinins have been demonstrated to play an important role in establishing seed size, decreasing tip kernel abortion, and increasing seed set during unfavorable environmental conditions.

Methods are therefore provided to decrease activity and/or level of CKX polypeptides in the developing female inflorescence, thereby elevating cytokinin levels and allowing developing seed to achieve their full genetic potential for size, minimizing tip kernel abortion, and buffering seed set during unfavorable environments. The methods further allow the plant to maintain and/or improve the flowering process during unfavorable environments.

In this embodiment, a variety of promoters could be used to direct the expression of a sequence capable of decreasing the level and/or activity of the CKX polypeptide. In one method, a stress insensitive/lag phase/developing kernel-preferred promoter is used. By “insensitive to stress” is intended that the expression level of a sequence operably linked to the promoter is not altered or only minimally altered under stress conditions. Such promoters are known in the art and include Zag2.1 (Schmidt et al. (1993) Plant Cell 5:729-737, Genbank Accession No. X80206), ZmCkx1-2 promoter (U.S. patent application Ser. Nos. 10/109,488 and 11/074,144), ZmCkx2 promoter (SEQ ID NO:13), ZmCkx3 promoter (SEQ ID NO:14), ZmCkx4 promoter (SEQ ID NO:15), ZmCkx5 promoter (SEQ ID NO:16), ZmCkx6 promoter (see SEQ ID NO: 51), any other CKX promoter, and mzE40 (Zm40) (U.S. Pat. No. 6,403,862 and WO01/2178). Alternatively, a stress-responsive promoter may be used, such as rd29a (Yamaguchi-Shinozaki et al. (1993) Mol. Gen. Genetics 236:331-334).

Methods to assay for an increase in seed set during abiotic stress are known in the art. For example, plants having the reduced CKX activity can be monitored under various stress conditions and compared to controls plants. For instance, the plant having the reduced CKX activity can be subjected to various degrees of stress during flowering and seed set. Under identical conditions, the genetically modified plant having the reduced CKX activity will have a higher number of developing kernels than will a wild type (non-transformed) plant.

Accordingly, the present invention further provides plants having increased yield or maintained yield and/or an increased or maintained flowering process during periods of abiotic stress (e.g. drought, salt, heavy metals, temperature, etc). In some embodiments, the plants having an increased or maintained yield during abiotic stress have a decreased level/activity of the CKX polypeptide of the invention. In other embodiments, the plant comprises a CKX nucleotide sequence of the invention operably linked to a promoter that drives expression in the plant cell. In certain embodiments, such plants have stably incorporated into their genome a nucleic acid molecule comprising a CKX nucleotide sequence of the invention operably linked to a promoter that drives expression in the plant cell.

VIII Modulating Pathogen Resistance

Methods for modulating pathogen resistance in a plant are provided. Plant pathogens can produce cytokinins (Mills et al. (1978) Physiol Plant Pathol 13:73-80 and Angra et al. (1990) Mycopathologia 109:177-182). Accordingly, increasing CKX activity in a plant or plant part can increase the plant's resistance to the pathogen. See, for example, Bilyeu et al. (2001) Plant Physiol. 125:378-386. Thus, compositions and methods for inducing resistance in a plant to plant pests are provided. In specific embodiments, the CKX polypeptide is provided to the developing seed and thereby increases the pathogen resistance of the seed. Accordingly, the compositions and methods are also useful in protecting plants against fungal pathogens, viruses, nematodes, insects and the like.

By “disease resistance” is intended that the plants avoid the disease symptoms that are the outcome of plant-pathogen interactions. That is, pathogens are prevented from causing plant diseases and the associated disease symptoms, or alternatively, the disease symptoms caused by the pathogen are minimized or lessened. By “antipathogenic compositions” is intended that the compositions of the invention have antipathogenic activity and thus are capable of suppressing, controlling, and/or killing the invading pathogenic organism. An antipathogenic composition of the invention will reduce the disease symptoms resulting from pathogen challenge by at least about 2% to at least about 6%, at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

The method for increasing pathogen resistance in a plant comprises increasing the level or activity of the CKX polypeptides of the invention. In specific methods, a CKX sequence of the invention is provided. A CKX nucleotide sequence can be provided by introducing into the plant a polynucleotide comprising a CKX nucleotide sequence of the invention, expressing the CKX sequence, and thereby increasing pathogen resistance in the plant. In certain embodiments, the CKX nucleotide construct introduced into the plant is stably incorporated into the genome of the plant.

As discussed above, one of skill will recognize the appropriate promoter to use to increase pathogen resistance in the plant. Exemplary promoters for this embodiment include constitutive promoters, tissue-preferred promoters, pathogen-inducible promoters, and seed-preferred promoters.

Assays that measure antipathogenic activity are commonly known in the art, as are methods to quantitate disease resistance in plants following pathogen infection. See, for example, U.S. Pat. No. 5,614,395, herein incorporated by reference. Such techniques include measuring over time the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

Pathogens of the invention include, but are not limited to, viruses or viroids, bacteria, insects, nematodes, fungi, and the like. Viruses include any plant virus, for example, tobacco or cucumber mosaic virus, ringspot virus, necrosis virus, or maize dwarf mosaic virus.

IX. Method of Use for CKX Promoter Polynucleotides

The polynucleotides comprising the CKX promoters disclosed in the present invention, as well as variants and fragments thereof, are useful in the genetic manipulation of any host cell, preferably plant cell, when assembled with a DNA construct such that the promoter sequence is operably linked to a nucleotide sequence comprising a polynucleotide of interest. In this manner, the CKX promoter polynucleotides of the invention are provided in expression cassettes along with a polynucleotide sequence of interest for expression in the host cell of interest. As discussed in Example 2 below, the CKX promoter sequences of the invention are expressed in a variety of tissues and thus the promoter sequences can find use in regulating the temporal and/or the spatial expression of polynucleotides of interest.

Synthetic hybrid promoter regions are known in the art. Such regions comprise upstream promoter elements of one polynucleotide operably linked to the promoter element of another polynucleotide. In an embodiment of the invention, heterologous sequence expression is controlled by a synthetic hybrid promoter comprising the CKX promoter sequences of the invention, or a variant or fragment thereof, operably linked to upstream promoter element(s) from a heterologous promoter. Upstream promoter elements that are involved in the plant defense system have been identified and may be used to generate a synthetic promoter. See, for example, Rushton et al. (1998) Curr. Opin. Plant Biol. 1:311-315. Alternatively, a synthetic CKX promoter sequence may comprise duplications of the upstream promoter elements found within the CKX promoter sequences.

It is recognized that the promoter sequence of the invention may be used with its native CKX coding sequences. A DNA construct comprising the CKX promoter operably linked with its native CKX gene may be used to transform any plant of interest to bring about a desired phenotypic change, such as modulating cytokinin levels, modulating root, shoot, leaf, floral, and embryo development, stress tolerance, and any other phenotype described elsewhere herein.

The promoter nucleotide sequences and methods disclosed herein are useful in regulating expression of any nucleotide sequence in a host plant in order to vary the phenotype of a plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic traits and characteristics such as yield and heterosis increases, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism, as well as those affecting kernel size, sucrose loading, and the like.

In one embodiment, sequences of interest improve plant growth and/or crop yields. In more specific embodiments, expression of the nucleotide sequence of interest improves the plant's response to stress induced under high density growth conditions. For example, sequences of interest include agronomically important genes that result in improved primary or lateral root systems. Such genes include, but are not limited to, nutrient/water transporters and growth inducers. Examples of such genes include, but are not limited to, maize plasma membrane H⁺-ATPase (MHA2) (Frias et al. (1996) Plant Cell 8:1533-44); AKT1, a component of the potassium uptake apparatus in Arabidopisis, (Spalding et al. (1999) J Gen Physiol 113:909-18); RML genes which activate cell division cycle in the root apical cells (Cheng et al. (1995) Plant Physiol 108:881); maize glutamine synthetase genes (Sukanya et al. (1994) Plant Mol Biol 26:1935-46) and hemoglobin (Duff et al. (1997) J. Biol. Chem 27:16749-16752, Arredondo-Peter et al. (1997) Plant Physiol. 115:1259-1266; Arredondo-Peter et al. (1997) Plant Physiol 114:493-500 and references cited therein); and isopentenyl transferase, or ipt (Strabala et al. (1989) Mol. Gen. Genet. 216:388-394, (Agrobacterium); U.S. patent applications 60/610,656 filed Sep. 17, 2004, and 60/637,230 filed Dec. 17, 2004 (maize); Takei et al. (2001) J. Biol. Chem. 276:26405-26410 (Arabidopsis); Zubko et al. (2002) Plant J. 29(6):797-808 (petunia); Sakano et al. (2004) Phytochem. 65:2439-2446 (hop); and GenBank accession XM_{—}477138 (rice, 2004)). The sequence of interest may also be useful in expressing antisense nucleotide sequences of genes that negatively affect root development.

Additional, agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. Pat. Nos. 5,703,049, 5,885,801, 5,885,802, and 5,990,389, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference); corn (Pedersen et al. (1986) J. Biol. Chem. 261:6279; Kirihara et al. (1988) Gene 71:359; both of which are herein incorporated by reference); and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), genes encoding proteins which break down glyphosate, or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in U.S. Pat. Nos. 5,703,049, 5,885,801, 5,885,802, and 5,990,389.

Commercial traits can also be encoded on a gene or genes that could increase for example, starch for ethanol production, or provide expression of proteins. Another important commercial use of transformed plants is the production of polymers and bioplastics such as described in U.S. Pat. No. 5,602,321. Genes such as β-Ketothiolase, PHBase (polyhydroxyburyrate synthase), and acetoacetyl-CoA reductase (see Schubert et al. (1988) J. Bacteriol. 170:5837-5847) facilitate expression of polyhyroxyalkanoates (PHAs).

Exogenous products include plant enzymes and products as well as those from other sources including procaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

All publications and patent herein referred to are hereby incorporated by reference to the same extent as if each was individually so incorporated.

The following examples are intended to illustrate but not limit the invention.

### Experimental

### EXAMPLE 1

### Sequence Analysis of CKX Sequence

The CKX polypeptides of the invention share sequence similarity with a number of CKX polypeptides. Table 1 summaries the sequence relationships of ZmCkx2, 3, 4, and 5 with each other and also with known or putative CKX enzymes.

TABLE 1

| | ZmCkx1 | | ZmCkx2 | | ZmCkx3 | | ZmCkx4 | | ZmCkx5 | |
|---|---|---|---|---|---|---|---|---|---|---|
| Gene | Similarity | Identity: | Similarity | Identity: | Similarity | Identity: | Similarity | Identity: | Similarity | Identity: |
| ZmCkx1 | 100.0 | 100.0 | | | | | | | | |
| ZmCkx2 | 55.8 | 46.6 | 100.0 | 100.0 | | | | | | |
| ZmCkx3 | 60.6 | 53.8 | 56.2 | 47.7 | 100.0 | 100.0 | | | | |
| ZmCkx4 | 53.4 | 47.0 | 53.3 | 47.1 | 52.2 | 45.0 | 100.0 | 100.0 | | |
| ZmCkx5 | 50.1 | 41.2 | 53.9 | 45.1 | 51.0 | 42.7 | 52.6 | 45.4 | 100.0 | 100.0 |

| Gene | CKO2 | | CKO3 | | OsCkx3 | | OsCkx6 | |
|---|---|---|---|---|---|---|---|---|
| | (CAE55200) | | (CAE55201) | | (NP_922039) | | (BAD09964) | |
| Gene | Similarity | Identity: | Similarity | Identity: | Similarity | Identity: | Similarity | Identity: |
| ZmCkx1 | 55.8 | 46.6 | 55.5 | 46.6 | 53.3 | 45.5 | 53.9 | 46.7 |
| ZmCkx2 | 100.0 | 100.0 | 96.0 | 94.2 | 54.3 | 43.7 | 53.0 | 46.8 |
| ZmCkx3 | 56.2 | 47.7 | 56.2 | 48.1 | 50.7 | 43.1 | 52.4 | 45.2 |
| ZmCkx4 | 53.3 | 47.1 | 52.6 | 46.0 | 51.9 | 42.9 | 84.4 | 82.0 |
| ZmCkx5 | 53.9 | 45.1 | 52.3 | 44.4 | 82.9 | 78.5 | 50.2 | 42.9 |

Based on alignments with GAP using BLOSUM62 amino acid substitution matrix.

Reference: Henikoff, S. and Henikoff, J. G. (1992). Amino acid substitution matrices from protein blocks. Proc. Natl. Acad. Sci. USA 89: 10915-10919

The amino acid alignment of ZmCkx1 (SEQ ID NO:33), ZmCkx2 (SEQ ID NO:3), ZmCkx3 (SEQ ID NO:6), ZmCkx4 (SEQ ID NO:9), ZmCkx5 (SEQ ID NO:12), and ZmCkx6 (SEQ ID NO: 53), along with the consensus sequence (SEQ ID NO:34) is provided in FIGS. 1A and B.

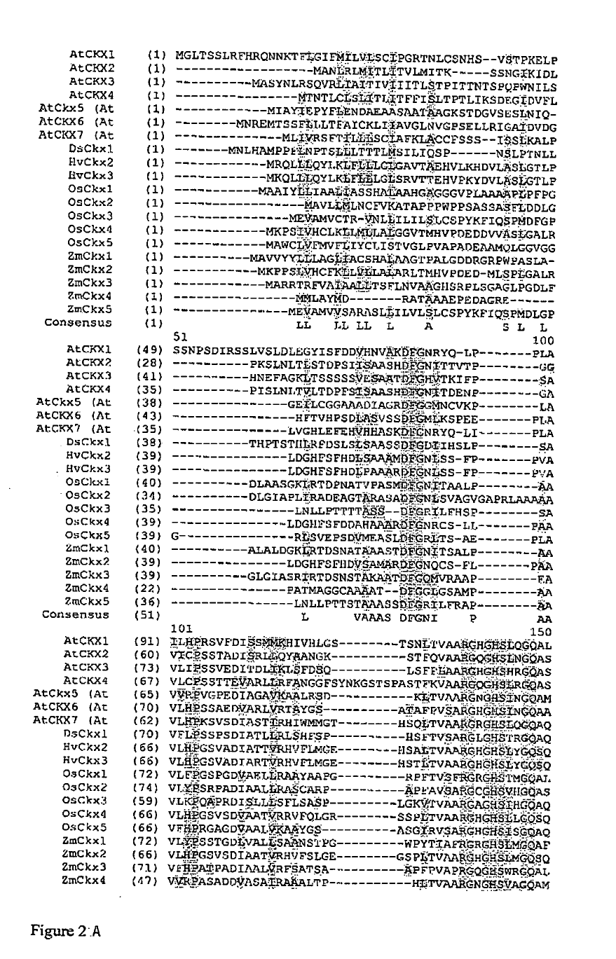

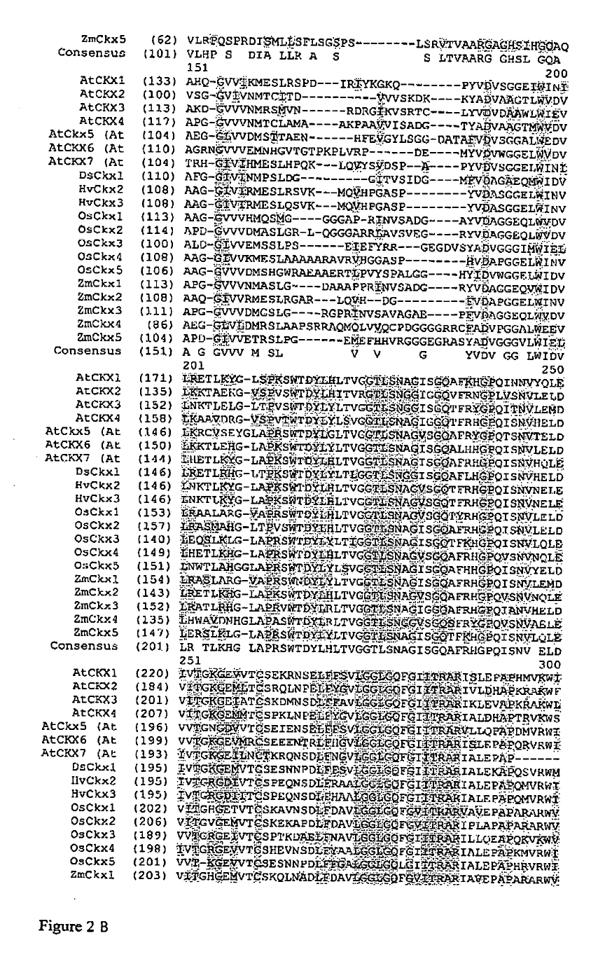

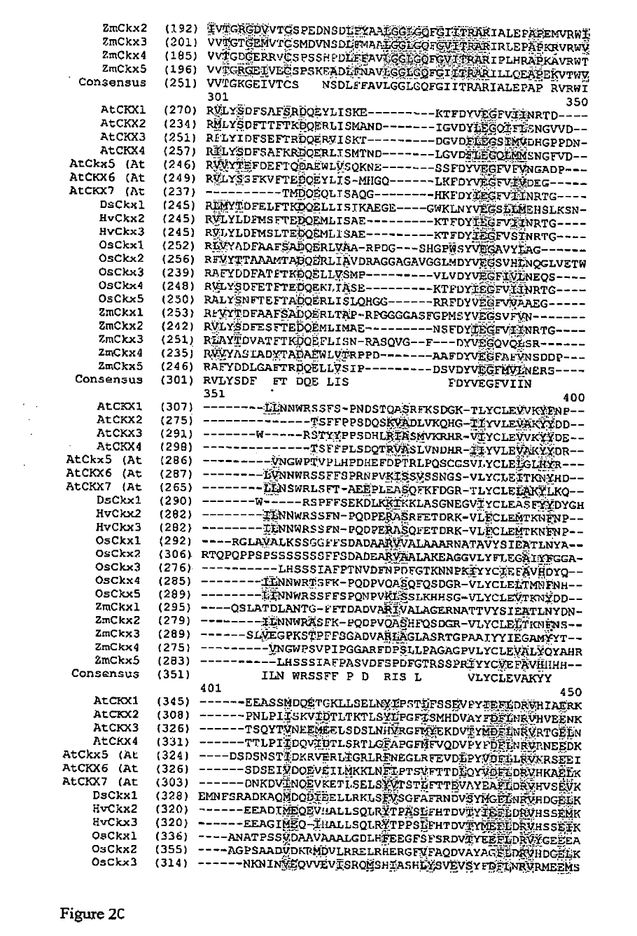

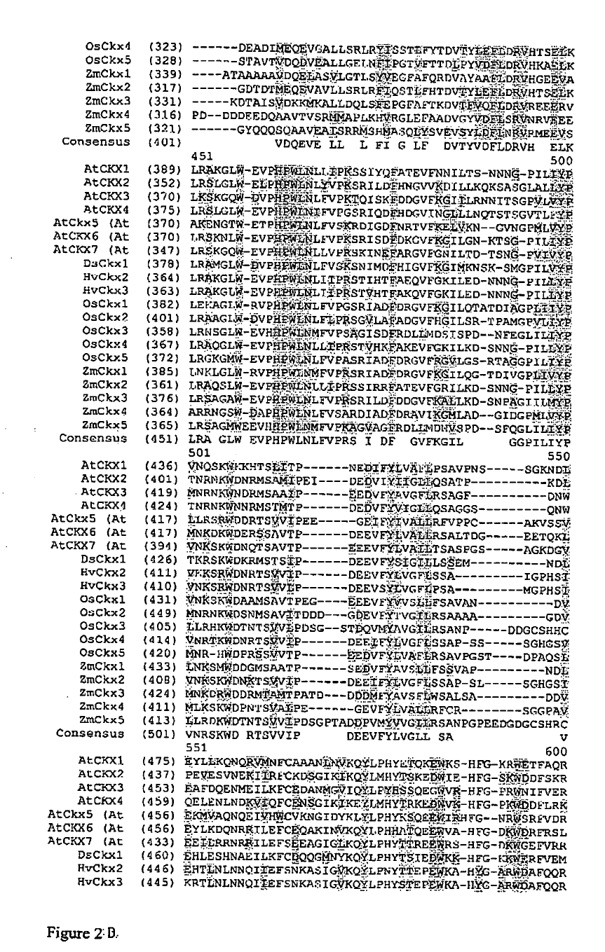

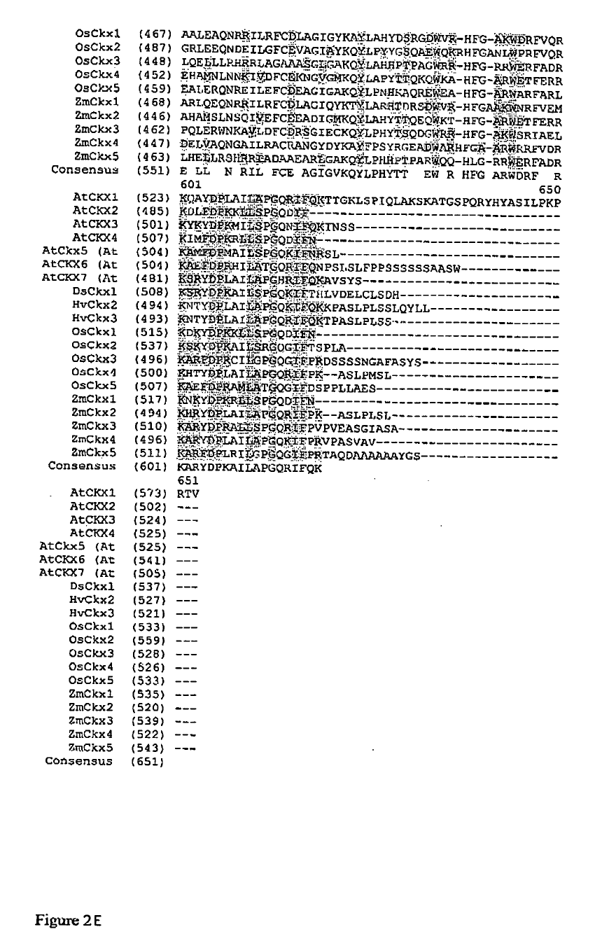

The amino acid alignment of the CKX polypeptides of the invention with other known CKX polypeptides is provided in FIG. 2A-F. Specifically, the alignment provides the sequence relationship of AtCkx1 (SEQ ID NO:35), AtCkx2 (SEQ ID NO:36), AtCkx3 (SEQ ID NO:37), AtCkx4 (SEQ ID NO:38), AtCkx5 (SEQ ID NO:39), AtCkx6 (SEQ ID NO:40), AtCkx7 (SEQ ID NO:41), DsCkx1 (SEQ ID NO:42), HvCkx2 (SEQ ID NO:43), HvCkx3 (SEQ ID NO:44), OsCkx1 (SEQ ID NO:45), OsCkx2 (SEQ ID NO:46), OsCkx3 (SEQ ID NO:47), OsCkx4 (SEQ ID NO:48), OsCkx5 (SEQ ID NO:49), ZmCkx1 (SEQ ID NO:33), ZmCkx2 (SEQ ID NO:3), ZmCkx3 (SEQ ID NO:6), ZmCkx4 (SEQ ID NO:10) and ZmCkx5 (SEQ ID NO:14). A consensus sequence is provided in SEQ ID NO:50.

The CKX polypeptides of the invention contain a predicted FAD-binding domain (PFAM Accession No. PF01565). As shown in FIG. 9, the FAD-binding domain is found from amino acid 63 to 220 of ZmCkx2, from amino acid 68 to 229 of ZmCkx3, from amino acid 44 to 213 of ZmCkx4 and from amino acid 59 to 224 of ZmCkx5.

An analysis of the subcellular location of the CKX polypeptides of the invention was also performed. The results of these analyses are set forth below.

A. Analysis of ZmCkx2:

A signal prediction was run using ProtComp trained onto plants. The results for ZmCkx2 follow and predict that the ZmCkx2 polypeptide is extracellularly localized.

ProtComp Version 5. Identifying sub-cellular location (Plants)

Seq name: ZmCkx2 519
Significant similarity in Location DB - Location: Extracellular
(Secreted)
Database sequence: AC = Q9T0N8 Location: Extracellular (Secreted)
DE Cytokinin oxidase 1 precursor (EC
Score = 11145, Sequence length = 534, Alignment length = 392
Predicted by Neural Nets - Plasma membrane with score 0.9
******** Transmembrane segments are found: .−325 : 337+.
Integral Prediction of protein location: Membrane bound Extracellular
(Secreted) with score 4.3

| Location weights: | LocDB | PotLocDB | Neural Nets | Integral |
|---|---|---|---|---|
| Nuclear | 0.0 | 0.0 | 0.74 | 0.74 |
| Plasma membrane | 0.0 | 0.0 | 0.92 | 0.92 |
| Extracellular | 11145.0 | 9230.0 | 0.81 | 4.31 |
| Cytoplasmic | 0.0 | 0.0 | 0.64 | 0.64 |
| Mitochondrial | 0.0 | 0.0 | 0.76 | 0.76 |
| Chloroplast | 0.0 | 0.0 | 0.73 | 0.73 |
| Endoplasm. retic. | 0.0 | 0.0 | 0.77 | 0.77 |
| Peroxisomal | 0.0 | 0.0 | 0.76 | 0.76 |

SPScan in SeqWeb
1. 1 mkppslvhcfkllvllalarltmh\hat{ }vp 26
Score: 7.7
Probability: 7.225E−01
SP Length: 24
McGeoch scan succeeded:
Charged-region statistics:
Length: 11 Charge: 2
Hydrophobic-region statistics:
Length: 8 Offset: 12 Total hydropathy: 62.3
Maximum 8-residue hydropathy: 62.3, starting at 13

B. Analysis of ZmCkx3:

A signal prediction was run using ProtComp trained onto plants. The results for ZmCkx3 follow and predict that the ZmCkx3 polypeptide is extracellularly localized.

ProtComp Version 5. Identifying sub-cellular location (Plants)

Seq name: ZmCkx3 538
Significant similarity in Location DB - Location: Extracellular
(Secreted)
Database sequence: AC = Q9T0N8 Location: Extracellular (Secreted)
DE Cytokinin oxidase
Score = 13520, Sequence length = 534, Alignment length = 500
Predicted by Neural Nets - Plasma membrane with score 1.3
Integral Prediction of protein location: Extracellular (Secreted)
with score 5.3

| Location weights: | LocDB | PotLocDB | Neural Nets | Integral |
|---|---|---|---|---|
| Nuclear | 0.0 | 0.0 | 1.18 | 1.18 |
| Plasma membrane | 0.0 | 0.0 | 1.32 | 1.32 |
| Extracellular | 13520.0 | 11200.0 | 1.07 | 5.32 |
| Cytoplasmic | 0.0 | 0.0 | 0.72 | 0.72 |
| Mitochondrial | 0.0 | 0.0 | 0.98 | 0.98 |
| Chloroplast | 0.0 | 0.0 | 0.71 | 0.71 |
| Endoplasm. retic. | 0.0 | 0.0 | 0.56 | 0.56 |
| Peroxisomal | 0.0 | 0.0 | 0.42 | 0.42 |

C. Analysis of ZmCkx4:

A signal prediction was run using ProtComp trained onto plants. The results for ZmCkx4 follow and predict that the ZmCkx4 polypeptide is extracellularly localized.

ProtComp Version 5. Identifying sub-cellular location (Plants)

Seq name: ZmCkx4 521
Significant similarity in Location DB - Location: Extracellular
(Secreted)
Database sequence: AC = Q9LTS3 Location: Extracellular (Secreted)
DE Cytokinin oxidase
Score = 10155, Sequence length = 523, Alignment length = 360
Predicted by Neural Nets - Plasma membrane with score 1.3
Integral Prediction of protein location: Extracellular (Secreted)
with score 4.4

| Location weights: | LocDB | PotLocDB | Neural Nets | Integral |
|---|---|---|---|---|
| Nuclear | 0.0 | 0.0 | 1.18 | 1.18 |
| Plasma membrane | 0.0 | 0.0 | 1.32 | 1.32 |
| Extracellular | 10155.0 | 9925.0 | 1.07 | 4.43 |
| Cytoplasmic | 0.0 | 0.0 | 0.72 | 0.72 |
| Mitochondrial | 0.0 | 0.0 | 0.95 | 0.95 |
| Chloroplast | 0.0 | 0.0 | 0.71 | 0.71 |
| Endoplasm. retic. | 0.0 | 0.0 | 0.56 | 0.56 |
| Peroxisomal | 0.0 | 0.0 | 0.42 | 0.42 |

D. Analysis of ZmCkx5:

A signal prediction was run using ProtComp trained onto plants. The results for ZmCkx5 follow and predict that the ZmCkx5 polypeptide is extracellularly localized.

ProtComp Version 5. Identifying sub-cellular location (Plants)

Seq name: ZmCkx5 542
Significant similarity in Location DB - Location: Extracellular
(Secreted)
Database sequence: AC = Q9LTS3 Location: Extracellular (Secreted)
DE Cytokinin oxidase
Score = 9405, Sequence length = 523, Alignment length = 390
Predicted by Neural Nets - Plasma membrane with score 1.3
Integral Prediction of protein location: Extracellular (Secreted)
with score 4.3

| Location weights: | LocDB | PotLocDB | Neural Nets | Integral |
|---|---|---|---|---|
| Nuclear | 0.0 | 0.0 | 1.18 | 1.18 |
| Plasma membrane | 0.0 | 0.0 | 1.32 | 1.32 |
| Extracellular | 9405.0 | 10020.0 | 1.08 | 4.28 |
| Cytoplasmic | 0.0 | 0.0 | 0.72 | 0.72 |
| Mitochondrial | 0.0 | 0.0 | 0.98 | 0.98 |
| Chloroplast | 0.0 | 0.0 | 0.71 | 0.71 |
| Endoplasm. retic. | 0.0 | 0.0 | 0.56 | 0.56 |
| Peroxisomal | 0.0 | 0.0 | 0.42 | 0.42 |

### EXAMPLE 2

### Expression Profiles of Cytokinin Oxidase Genes

Several cytokinin oxidase ESTs were identified and genomic sequences isolated from corresponding BAC clones. The corresponding genes were named ZmCkx2, ZmCkx3, ZmCkx4, ZmCkx5, and ZmCkx6. Expression profiles of the CKX sequences were studied using Northern blots and RT-PCR, and using a proprietary Lynx database (Lynx Therapeutics, Hayward Calif, USA; see, for example, Brenner et al., Nature Biotechnology (2000) 18:630-634).

A. Analysis of ZmCkx2

Northern analysis of ZmCkx2 was performed using ExpressHyb™ Hybridization Solution from BD Biosciences Clontech (Palo Alto, Calif.) with a final wash in 0.1×SSC, 0.1% SDS at 65° C. for 20 minutes.

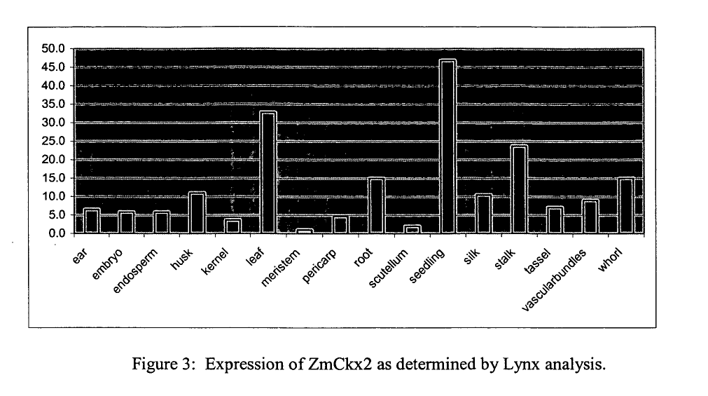

A tight relationship exists between Lynx and Northern data for ZmCkx2. This provided confidence when the Lynx database was mined for ZmCkx2 expression in various plant parts. For example, both Northern and Lynx analyses showed that ZmCkx2 had a 2-fold increase in expression in leaf discs incubated with 10 μM benzyladenine (a synthetic cytokinin). Lynx data in FIG. 3 show that expression is highest in leaves, stalk, whorl, roots and seedlings. Similarly, Northern data indicated strongest signals from ear leaf and midrib tissues; intermediate levels in tassel, husk leaves, young leaves, stalk, and pulvini; and lower levels in cob and ovary tissue. Little to no ZmCkx2 activity was detected by Northern analysis of roots or silks.

In addition, analyses of the Lynx data revealed that expression of ZmCkx2 increases during root aging and is induced 4-fold in seedlings submitted to a freezing stress. In the stalk, expression is 3-fold higher in the pith than in the rind.

RT-PCR was performed to determine the expression profile of ZmCkx2 in various maize tissues. RT-PCR was performed on maize mature and seedling tissue employing the following PCR parameters: 94° C. for 45 sec, 60° C. for 1 min, 72° C. 3 min, for 30 cycles. ZmCkx2 expression was strongest in mature stalk tissue and in seedling leaf and mesocotyl. Weaker expression was noted in midribs and young and mature leaves of mature plants, as well as seedling roots. Similar RT-PCR studies were also performed during various stages of maize kernel development, including 0, 5, 10, 15, 20, 25, and 30 days after pollination. An expression peak was detected at 5 DAP.

A proprietary Agilent database (Agilent Technologies, Palo Alto, Calif.) was also analyzed to identify trends in ZmCkx2 expression. Tissues that showed the most dramatic differences in ZmCkx2 expression are from stalk. These samples were collected from the intemodal zone of the 3^{rd} or 4^{th} internode below the ear before and after flowering. It was found that ZmCkx2 expression goes up more than 10-fold in the stalk after flowering (Table 2).

TABLE 2

| Experiment | | | Fold | |
|---|---|---|---|---|
| Id | Experiment Name | Ratio | Change | P-value |
| 2340 | Pt#1 preflowering at 59 K vs Pt#2 postflowering at 59 K | 0.08 | −12.8 | 1.73E−27 |
| 2364 | Pt#2 preflowering at 27 K vs Pt#2 postflowering at 59 K | 0.09 | −11.3 | 7.77E−26 |
| 2349 | Pt#1 preflowering at 27 K vs Pt#3 postflowering at 59 K | 0.09 | −11.1 | 5.73E−23 |
| 2336 | Pt#3 preflowering at 59 K vs Pt#1 postflowering at 59 K | 0.09 | −10.7 | 7.05E−25 |
| 2345 | Pt#3 preflowering at 27 K vs Pt33 postflowering at 27 K | 0.1 | −10.3 | 3.67E−24 |
| 2332 | Pt#2 postflowering at 27 K vs Pt#1 preflowering at 59 K | 6.82 | 6.82 | 7.37E−17 |
| 2359 | Pt#1 postflowering at 59 K vs Pt#2 preflowering at 27 K | 9.16 | 9.16 | 6.71E−22 |
| 2368 | Pt#2 postflowering at 27 K vs Pt#3 preflowering at 59 K | 11.17 | 11.17 | 1.07E−25 |
| 2366 | Pt#1 postflowering at 27 K vs Pt#3 preflowering at 59 K | 11.78 | 11.78 | 2.04E−26 |
| 2333 | Pt#3 postflowering at 27 K vs Pt#1 preflowering at 27 K | 17.7 | 17.7 | 1.72E−31 |

Table 2 shows fold changes identified in stalk samples collected from the internodal zone of the 3rd or 4th internode below ear, before and after flowering. This increase in ZmCkx2 expression could be associated with the flowering process. An increase of cytokinin flux from roots to shoots is often regarded as a flowering signal and is consistent with previous findings that increased cytokinin levels induce ZmCkx1 and ZmCkx2 expression. ZmCkx2 expression was also found to increase an average of 10-fold during ear development. Thus, manipulation of ZmCkx2 expression may be useful in modulation of flowering time.
B. Analysis of ZmCkx3

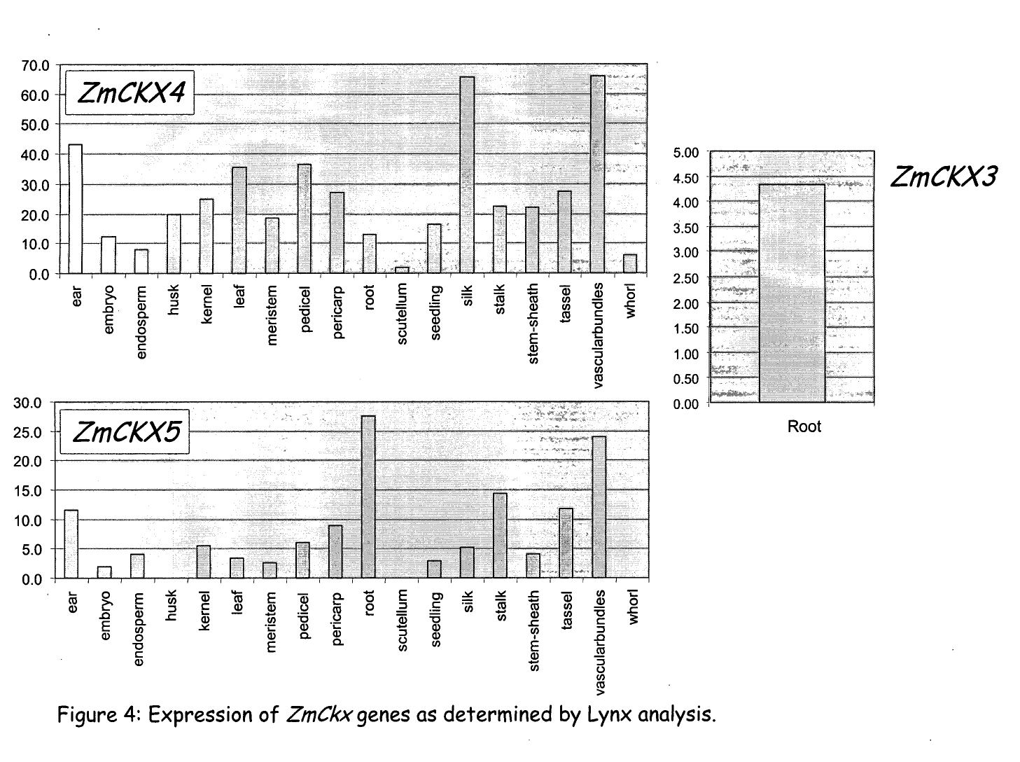

Expression of ZmCkx3 could not be detected using Northern blots. Mining of the Agilent and Lynx database confirmed that the gene is expressed at extremely low levels. The EST for ZmCkx3 came from a tassel library and it is believed that this gene could be tightly expressed in a particular cell type at a particular stage of tassel development. It remains possible that ZmCkx3 expresses during anther development at very low levels. The only tags from Lynx are from roots at an average of 4-5 ppm (See FIG. 4).

C. Analysis of ZmCkx4

Analysis of the Lynx database for ZmCkx4 showed low constitutive expression of the gene in most organs, with higher levels observed in ear, silk and vascular bundles as well as intermediate levels in leaf and pedicels (FIG. 4). Interestingly, in 15-20 mm ears, ZmCkx4 is expressed at higher levels at the base of the ear than at the ear tip. This stage of ear growth coincides with the appearance of silk structure on the ear, which, taken together with strong expression in the silk, suggests a role for this gene in silk development.

D. Analysis of ZmCkx5

Analysis of the Lynx database for ZmCkx5 showed highest levels of expression to be in root and vascular bundles. (See FIG. 4)

### EXAMPLE 3

### Identification of ZmCkx2 and ZmCkx4 TUSC Events

In order to better define the role of ZmCkx2 and ZmCkx4 in plant development, knockout mutants for these two genes were obtained. A TUSC summary follows for each sequence.

A. ZmCkx2 TUSC Summary

Two genomic sequences for cytokinin oxidase orthologues were provided for knockout screening. ZmCkx2 is a ˜3200 bp genomic sequence with five exons and four introns. Using this annotation, six PCR primers were designed across various intervals of the ZmCkx2 gene and then tested in control reactions against wild type maize (B73) gDNA. Primers were identified as 71936 (SEQ ID NO: 19), 71937 (SEQ ID NO: 20), 71938 (SEQ ID NO: 21), 71939 (SEQ ID NO: 22), 71940 (SEQ ID NO: 23), 71941 (SEQ ID NO: 24) and 9242 MuTIR (SEQ ID NO: 25). Verification and clean results were obtained for 71936+71937, 71940+71937, 71940+71941, 71940+71939, 71938+71941 and 71938+71939. No amplification results were observed for 71936+71941 and 71936+71939.

The 71936+71937 and 71938+71939 amplification products were cut out of the agarose gel, purified, and used as probes for hybridization. These two intervals effectively segment the ZmCKX2 gene into 5′ and 3′ halves for insertion screening. Primer sequences are shown below along with the expected and observed amplicon sizes for each primer combination.

TABLE 3

| Primer Pair | cDNA (bp) | observed (bp) |
|---|---|---|
| 71936 + 71937 | 798 | ˜800 |
| 71936 + 71941 | 1350 | No product |
| 71936 + 71939 | 1841 | No product |
| 71940 + 71937 | 245 | ˜250 |
| 71940 + 71941 | 797 | ˜800 |
| 71940 + 71939 | 1288 | ˜1300 |
| 71938 + 71941 | 310 | ˜300 |
| 71938 + 71939 | 801 | ˜800 |

The pooled TUSC population was screened with gene primers 71936, 71937, 71938, and 71939 each in combination with the Mutator TIR primer 9242. Results of the pool hybridizations were fair with some PCR-positive pools detected by hybridization. Overall, hybridization signals were cross-confirmed between the primers.

Pools were selected for fragment sizing analysis based on hybridization signal intensity and reproducibility of the pool dot blots. In this phase of the screen, sizes of target::Mu PCR products are determined by reamplification, electrophoresis, and Southern analysis. Fourteen positive pools for primer 71936, fifty-one positive pools for primer 71937, forty-four positive pools for primer 71939, and thirty-seven positive pools for 71938 were screened through fragment-sizing. A number of pools were identified with strong EtBr and Southern bands.

Eight pools were selected for individual analysis based on the putative Mutator insertion location within ZmCKX2, determined from the size data, and the overall quality of the hybridization signals throughout the screening process. The pools are shown in the table below, along with their size data. Each plate listed consists of individuals from two pools: those assayed in the sizing analysis (highlighted in bold type), as well as individuals from its companion pools. Individuals in the companion pools are often, but not necessarily, related to those in the targeted pools.

TABLE 4
71937

| Plate | Pools | Size (Bp) for Pool |
|---|---|---|
| PV03 60 | 119 and 120 | 350 |
| PV03 70 | 139∴ and 140 | 425 |
| PV03 71 | 141 and 142 | 600 |
| PV03 94 | 187 and 188 | 750 |
| PV03 118 | 235∴ and 236 | 750 |
| PV03 119 | 237 and 238 | 225 |
| PV03 159 | 317 and 318 | 350 |
| BT94 166 | 841∴ and 842 | 425 |

Individual DNAs were arrayed, and a dot blot screen conducted with 71936 and 71937. Note that these selections are focused on the best candidates from the 5′ half of the gene, targeting primarily the first large exon. In individual screens, PCR-positive individuals were identified for all of the targeted pools. To ensure germinal transmission of target::Mu alleles, F2 transmission testing was performed on thirty individual families harboring putative ZmCKX2::Mu alleles. F2 genomic DNA was isolated from dry kernels (5K/individual) and amplified with the appropriate primers. Template controls on these preps were also performed using the gene-specific pair 71936+71937.

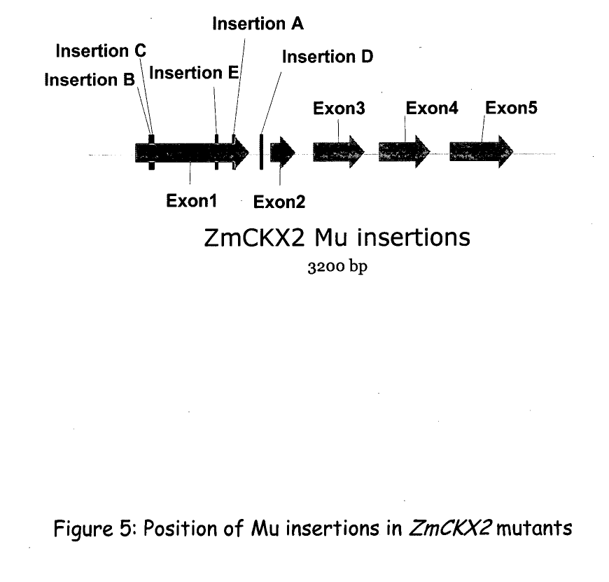

FIG. 5 provides a schematic of various Mu insertions in ZmCkx2 and ZmCkx4. Results indicate the genetic transmission of five ZmCkx2::Mu alleles.

1) Insertion A: This insertion is inherited uniquely by this F2 family in Pool 139. The insertion is cross-confirmed from both flanks of the insertion, producing strong EtBr and hybridization signals in F2 tests. The allele amplifies a ˜625 fragment with 71936+9242, cross-confirmed with a ˜375bp fragment using 71937+9242. This provides evidence for a knockout allele in the first exon of ZmCkx2, near nt 800 of the genomic reference sequence.

2) Insertion B: Several related sibling families inherit the same insertion allele, suggesting a pre-meiotic origin for this allele; a parental insertion would have been evident in many more positive families. Five strong positive individuals were subjected to F2 tests; all were positive for the insertion allele. This insertion is cross-confirmed by amplification from both flanks. The 71936+9242 combination produces a small product of ˜150 bp, and the 3′ flank primer pair 71937+9242 produces a fragment of ˜800 bp. The insertion site is thus predicted to be near the beginning of Exon I, and may be in the untranslated region. A Mu-suppressible phenotype may be one outcome of an insertion in this position.

3) Insertion C: This is a uniquely inherited Mu insertion in the 5′ end of ZmCkx2. The allele is of a distinct pedigree from that of Allele 2, yet it produces very similar PCR product sizes as those listed above from 5′ and 3′ flanks.

4) Insertion D: This is another uniquely inherited and cross-confirmed insertion in the 5′ end of the ZmCKX2 gene. This insertion produces fragments of ˜775 bp and ˜225 bp with 5′ (71936) and 3′ (71937) primer combinations, respectively. Based on the genomic annotation, this insertion occurs in Intron I of the gene, and thus may not provide a strong knockout allele. DNA sequence confirmation will be necessary to substantiate the expectations for this allele.

5) Insertion E: This is a uniquely inherited insertion, again cross-confirmed by amplification from both flanks of the insertion site. The allele produces strong EtBr and hybridization fragments of ˜525 bp with the 71936+9242 combination, and ˜475 bp with the 71937+9242 combination. This insertion position appears to squarely interrupt Exon I of the gene, and is perhaps the best candidate for a good null in the ZmCKX2 gene.

B. ZmCKX4 TUSC Summary

Like ZmCkx2, a complete genomic sequence for ZmCkx4 was provided to facilitate knockout screening. Alignments of the two genes were used, and known intron sequences identified to enable the design of primers specific for insertions in ZmCkx4. Following these analyses, six PCR primers were designed across various intervals of ZmCkx4 and tested in control pairs against wt maize (B73) gDNA. Primers were identified as 71942 (SEQ ID NO: 26), 71943 (SEQ ID NO: 27), 71944 (SEQ ID NO: 28), 71945 (SEQ ID NO: 29), 71946 (SEQ ID NO: 30), 71947 (SEQ ID NO: 31), and 9249 MuTIR (SEQ ID NO: 32). Verification and clean results were obtained solely for the 71944+71947 primer combination. Further screening targeted Exon IV.

For Exon IV screening, the 71944+71947 amplification product was cut out of the agarose gel, purified, and used as probe for hybridization. Primer sequences are shown below along with the expected and observed amplicon sizes for each primer combination.

TABLE 5

| Primer Pair | cDNA (bp) | observed by |
|---|---|---|
| 71942 + 71943 | 1575 | No product |
| 71942 + 71947 | 2072 | No product |
| 71942 + 71945 | 3075 | No product |
| 71946 + 71943 | 763 | No product |
| 71946 + 71947 | 1260 | No product |
| 71946 + 71945 | 2263 | No product |
| 71944 + 71947 | 448 | 450 |
| 71944 + 71945 | 1451 | No product |

The pooled TUSC population was screened with gene primers 71944 and 71947, each in combination with the Mutator TIR primer 9242. Results of the pool hybridizations were fair with some PCR-positive pools detected by hybridization: some signals were reproducible, and were cross-confirmed between the primers.

Pools were selected for fragment sizing analysis based on hybridization signal intensity and reproducibility of the pool dot blots. In this phase of the screen, sizes of target::Mu PCR products are determined by reamplification, electrophoresis, and Southern analysis. Forty-five positive pools for primer 71944 and seven positive pools for primer 71947 were screened through fragment-sizing. A number of pools were identified with strong EtBr and Southern bands.

Six pools were selected for individual analysis based on the putative Mutator insertion location within ZmCkx4, determined from the size-data, and the overall quality of the hybridization signals throughout the screening process. The pools are shown in the table below, along with their size data. Insertions detected outside the bounds of the primer interval are useful to expand the search for insertions beyond exon IV. Each plate listed consists of individuals from two pools: those assayed in the sizing analysis (highlighted in bold type), as well as individuals from its companion pools. Individuals in the companion pools are often, but not necessarily, related to those in the targeted pools.

TABLE 6
71944 71947

| Plate | Pools | Size (bp) for Pool |
|---|---|---|
| PV03 47 | 93 and 94 | 1800 |
| PV03 119 | 237 and 238 | 1550 |
| PV03 170 | 339 and 340 | 400; 225 |
| PV03 253 | 505 and 506 | 175 |
| BT94 19 | 547 and 548 | 1675, 775, 350 |
| BT94 96 | 705 and 706 | 1175, 350 |

Individual DNAs were arrayed, and a dot blot screen conducted with 71944 and 71947. PCR-positive individuals were identified for all of the targeted pools. To ensure germinal transmission of target::Mu alleles, F2 transmission testing was performed on thirty individual families harboring putative ZmCkx4::Mu alleles. F2 genomic DNA was isolated from dry kernels (5K/individual) and amplified with the appropriate primers. Template controls on these preps were also performed using the gene-specific pair 71944+71947.

FIG. 5 provides a schematic of various Mu insertions in ZmCkx4. Results indicate the genetic transmission of three ZmCKX4::Mu alleles.

1) Insertion A: This unique insertion allele is detected solely with primer 71947+9242, and produces a large fragment of >1600bp. This is a positive signal and likely represents an insertion into Exon I of the ZmCkx4 gene. Further characterization of this allele will include DNA sequencing and the design and testing of alternative 5′ primers.

2) Insertion B: A uniquely inherited insertion, this is cross-confirmed by amplification with both F and R primers from Exon IV. As such, this represents an excellent candidate for a knockout. The allele produces a strong product of ˜200bp with 71944+9242; cross-confirmed by the ˜400 bp product with 71947+9242. These primers may be useful for genotyping assays during propagation.

3) Insertion C: This is another uniquely inherited insertion into Exon IV. This insertion is near that of Allele 2. The insertion produces a small ˜175 bp product with the 71944+9242 combination and is cross-confirmed by a ˜425 bp product with the right flank combination 71947+9242.

All three of these alleles are excellent candidates for ZmCkx4 knockouts.

### EXAMPLE 4

### Expression of ZmCkx2 Modulates Plant Development

A DNA construct comprising ZmCkx2 operably linked to the ubiquitin promoter was introduced into maize plants as outlined in Zhao et al. (1998) Maize Genetics Corporation Newsletter 72:34-37, herein incorporated by reference.

Maize plants comprising a plasmid containing the ZmCkx2 sequence operably linked to a ubiquitin promoter were obtained. As a control, a non-cytokinin-related construct was also introduced into maize plants using the transformation method outlined above. Northern analysis indicated elevated levels of ZmCkx2 expression in transgenic events. The phenotypes of these transgenic maize plants having an elevated level of the ZmCkx2 polypeptide were further studied.

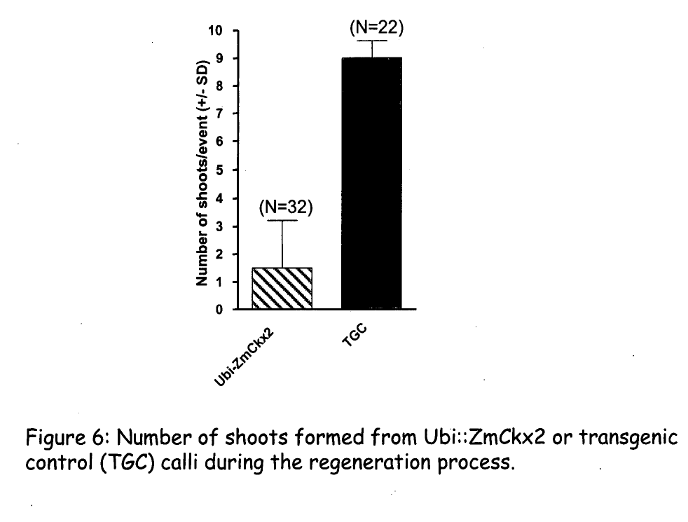

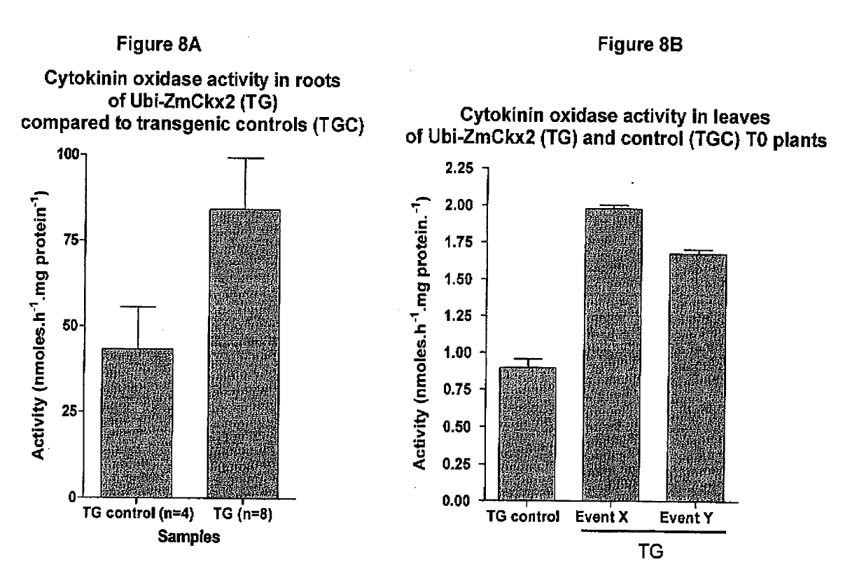

Callus cultures of the transgenic maize tissue produced significantly more roots and only one-sixth as many shoots as control plants during the regeneration process. (See FIG. 6) In addition, transgenic roots cultured in vitro and leaves of T0 plants in the greenhouse showed a 2-fold increase in cytokinin oxidase activity. (See FIG. 8)

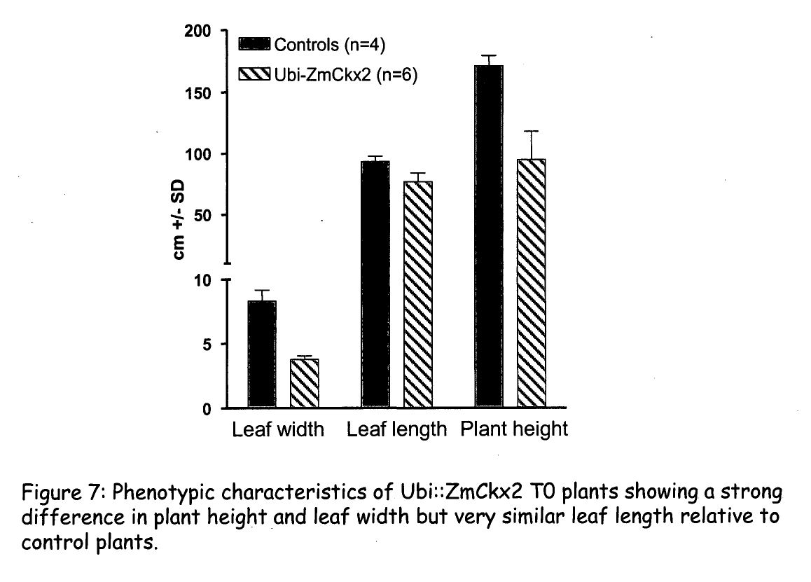

Plants growing in the greenhouse and expressing the ZmCkx2 sequence at high levels showed a phenotype typical of plants with lower cytokinin levels, including developmental problems as shorter plants with thinner leaves and a green/gray color. These differences were evident through the vegetative growth period. Out of 23 plants expressing the Ubi:ZmCkx2 sequence, 6 transgenic plants appeared to be of normal size, 8 transgenic plants displayed a medium size, 6 transgenic plants were small but viable, and 3 transgenic plants were very small. FIG. 7 provides data as to plant height, leaf length, and leaf width of transgenic plants compared to controls. Tassels of certain Ubi:ZmCkx2 plants lacked spikelets but generated silks capable of setting seed.

### EXAMPLE 5

### Assaying for Cytokinin Oxidase Activity

The level of cytokinin oxidase activity in the maize plant generated in Example 4 was measured. The assay to determine the level of cytokinin oxidase activity was carried out as described in Brugiere et al. (2003) Plant Physiol. 132:1228-1240, herein incorporated by reference.

As demonstrated in FIG. 8A, cytokinin oxidase activity in roots of transgenic plants is significantly higher than cytokinin oxidase activity in roots of control plants. In addition, as demonstrated in FIG. 8B, cytokinin oxidase activity in leaves is higher in plants expressing ZmCkx2 than in the control plants.

### EXAMPLE 6

### Maintaining or Increasing Seed Set During Stress

Immature maize embryos from greenhouse donor plants are bombarded with a plasmid designed to achieve post-transcriptional gene silencing (PTGS) with an appropriate promoter. For example, the plasmid may comprise the ZmCkx2 promoter (SEQ ID NO:13) operably linked to a sequence encoding a hairpin structure corresponding to the CDS of the ZmCkx2 polynucleotide (SEQ ID NO:2). The plasmid may also contain the selectable marker gene PAT (Wohlleben et al. (1988) Gene 70:25-37), which confers resistance to the herbicide Bialaphos. Transformation is performed as follows. Media recipes follow below.

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5 cm target zone in preparation for bombardment.

A plasmid vector is made comprising the ZmCkx2 promoter sequence operably linked to a sequence encoding a hairpin structure corresponding to the CDS of the ZmCkx2 polynucleotide. This plasmid DNA plus plasmid DNA containing a PAT selectable marker is precipitated onto 1.1 μm (average diameter) tungsten pellets using a CaCl₂ precipitation procedure as follows: 100 μl prepared tungsten particles in water; 10 μl (1 μg) DNA in Tris EDTA buffer (1 μg total DNA); 100 μl 2.5 M CaCl₂; and, 10 μl 0.1 M spermidine.

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

The sample plates are bombarded at level #4 in particle gun #HE34-1 or #HE34-2. All samples receive a single shot at 650 PSI, with a total of ten aliquots taken from each tube of prepared particles/DNA.

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored under various stress conditions and compared to control plants. The maintenance of or an increase in seed set during an abiotic stress episode is monitored.

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Geirite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-l H₂O); and 0.85 mg/l silver nitrate and 3.0 mg/l bialaphos(both added after sterilizing the medium and cooling to room temperature).

Plant regeneration medium (288J) comprises 4.3 g/l MS salts (GIBCO 11117-074), 5.0 ml/I MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### EXAMPLE 7

### Modulating Root Development

For Agrobacterium-mediated transformation of maize with the ZmCkx4 sequence operably linked to the CRWAQ81 root-preferred promoter::ADH intron, the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the zmCkx4 to at least one cell of at least one of the immature embryos (step 1: the infection step). In this step the immature embryos are immersed in an Agrobacterium suspension for the initiation of inoculation. The embryos are co-cultured for a time with the Agrobacterium (step 2: the co-cultivation step). The immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). The immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). The immature embryos are cultured on solid medium with a selective agent resulting in the selective growth of transformed cells. The callus is then regenerated into plants (step 5: the regeneration step), and calli grown on selective medium are cultured on solid medium to regenerate the plants.

Plants are monitored and scored for a modulation in root development. The modulation in root development includes monitoring for enhanced root growth of one or more root parts including the primary root, lateral roots, adventitious roots, etc. Methods of measuring such developmental alterations in the root system are known in the art. See, for example, U.S. application No. 2003/0074698 and Werner et al. (2001) PNAS 18:10487-10492, both of which are herein incorporated by reference.

### EXAMPLE 8

### Soybean Embryo Transformation

Soybean embryos are bombarded with a plasmid containing the ZmCkx3 sequence operably linked to a root-preferred promoter. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the ZmCkx2 sequence operably linked to the root-preferred promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl, of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100. psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### EXAMPLE 9

### Variants of CKX Sequences

A. Variant Nucleotide Sequences of CKX (SEQ ID NO: 2, 5, 8, 11, 52, 54, or 55) That Do Not Alter the Encoded Amino Acid Sequence

The CKX nucleotide sequences set forth in SEQ ID NO: 2, 5, 8, 11, 52, 54, and 55 are used to generate variant nucleotide sequences having the nucleotide sequence of the open reading frame with about 70%, 75%, 80%, 85%, 90%, and 95% nucleotide sequence identity when compared to the starting unaltered ORF nucleotide sequence of the corresponding SEQ ID NO. These functional variants are generated using a standard codon table. While the nucleotide sequence of the variants are altered, the amino acid sequence encoded by the open reading frames do not change.

B. Variant Amino Acid Sequences of CKX Polypeptides

Variant amino acid sequences of the CKX polypeptides are generated. In this example, one amino acid is altered. Specifically, the open reading frames set forth in SEQ ID NOS: 3, 6, 9, 12, or 53 are reviewed to determined the appropriate amino acid alteration. The selection of the amino acid to change is made by consulting the protein alignment (with the other orthologs and other gene family members from various species). See FIGS. 1 and 2. An amino acid is selected that is deemed not to be under high selection pressure (not highly conserved) and which is rather easily substituted by an amino acid with similar chemical characteristics (i.e., similar functional side-chain). Using the protein alignment set forth in FIGS. 1 and/or 2, an appropriate amino acid can be changed. Once the targeted amino acid is identified, the procedure outlined in Example 9A is followed. Variants having about 70%, 75%, 80%, 85%, 90%, and 95% sequence identity to SEQ ID NO:3, 6, 9, 12 or 53 are generated using this method.

C. Additional Variant Amino Acid Sequences of CKX Polypeptides

In this example, artificial protein sequences are created having 80%, 85%, 90%, and 95% identity relative to the reference protein sequence. This latter effort requires identifying conserved and variable regions from the alignment set forth in FIGS. 1 and 9 and then the judicious application of an amino acid substitutions table. These parts will be discussed in more detail below.

Largely, the determination of which amino acid sequences are altered is made based on the conserved regions among CKX protein or among the other CKX polypeptides. See FIGS. 1, 2, and 9. Based on the sequence alignment, the various regions of the CKX polypeptide that can likely be altered are represented in lower case letters, while the conserved regions are represented by capital letters. It is recognized that conservative substitutions can be made in the conserved regions below without altering function. In addition, one of skill will understand that functional variants of the CKX sequence of the invention can have minor non-conserved amino acid alterations in the conserved domain.

Artificial protein sequences are then created that are different from the original in the intervals of 80-85%, 85-90%, 90-95%, and 95-100% identity. Midpoints of these intervals are targeted, with liberal latitude of plus or minus 1%, for example. The amino acids substitutions will be effected by a custom Perl script. The substitution table is provided below in Table 8.

TABLE 8
Substitution Table

| | Strongly | Rank of | |
|---|---|---|---|
| | Similar and | Order | |
| Amino | Optimal | to | |
| Acid | Substitution | Change | Comment |
| I | L, V | 1 | 50:50 substitution |
| L | I, V | 2 | 50:50 substitution |
| V | I, L | 3 | 50:50 substitution |
| A | G | 4 | |
| G | A | 5 | |
| D | E | 6 | |
| E | D | 7 | |
| W | Y | 8 | |
| Y | W | 9 | |
| S | T | 10 | |
| T | S | 11 | |
| K | R | 12 | |
| R | K | 13 | |
| N | Q | 14 | |
| Q | N | 15 | |
| F | Y | 16 | |
| M | L | 17 | First methionine cannot change |
| H | | Na | No good substitutes |
| C | | Na | No good substitutes |
| P | | Na | No good substitutes |

First, any conserved amino acids in the protein that should not be changed is identified and “marked off” for insulation from the substitution. The start methionine will of course be added to this list automatically. Next, the changes are made.

H, C, and P are not changed in any circumstance. The changes will occur with isoleucine first, sweeping N-terminal to C-terminal. Then leucine, and so on down the list until the desired target it reached. Interim number substitutions can be made so as not to cause reversal of changes. The list is ordered 1-17, so start with as many isoleucine changes as needed before leucine, and so on down to methionine. Clearly many amino acids will in this manner not need to be changed. L, I and V will involve a 50:50 substitution of the two alternate optimal substitutions.

The variant amino acid sequences are written as output. Perl script is used to calculate the percent identities. Using this procedure, variants of the CKX polypeptides are generating having about 80%, 85%, 90%, and 95% amino acid identity to the starting unaltered ORF nucleotide sequence of SEQ ID NO:3, 6, 9, 12, or53.

### EXAMPLE 10

### Downregulation of Cytokinin Catabolism

The promoters of the present invention can be used in constructs designed to downregulate cytokinin oxidase activity. For example, certain embodiments comprise a construct comprising a segment of an endogenous cytokinin oxidase promoter such that, upon expression, self-hybridization of the RNA results in formation of hairpin RNA (hpRNA), resulting in transcriptional gene silencing of the native cytokinin oxidase gene. Thus, the embodiment comprises a nucleotide sequence which, when expressed in a cell, forms a hairpin RNA molecule (hpRNA), which suppresses (i.e., reduces or eliminates) expression of the endogenous cytokinin oxidase gene from its endogenous promoter. The ability of hpRNAs to suppress expression of a gene has been described (see, e.g., Matzke et al. (2001) Curr. Opin. Genet. Devel. 11:221-227; Scheid et al. (2002) Proc. Natl. Acad. Sci., USA 99:13659-13662; Waterhouse and Helliwell (2003) Nature Reviews Genetics 4:29-38; Aufsaftz et al (2002) Proc. Nat'l. Acad. Sci. 99(4):16499-16506; Sijen et al., Curr. Biol. (2001) 11:436-440).

The promoter which is operably linked to the nucleotide sequence encoding the hpRNA can be any promoter that is active in plant cells, particularly a promoter that is active (or can be activated) in reproductive tissues of a plant. As such, the promoter can be, for example, a constitutively active promoter, an inducible promoter, a tissue-specific promoter, a tissue-preferred promoter, a developmental stage specific promoter, or a developmental stage preferred promoter.

A hairpin may target a single promoter or may target two or more promoters by means of a single transcribed RNA. The hairpin-encoding region may be located in any appropriate position within the construct, such as within an intron of an encoded gene or within 5′ or 3′ non-coding regions, or may be the sole expressed element of the construct.

Methods for preparing said constructs and transforming plants may be as previously described (for example, see Cigan et al., Sex Plant Reprod. 14:135-142, 2001).

Said construct for downregulating cytokinin oxidase expression may be used in combination with other constructs or methods, such as those which result in increased cytokinin biosynthesis activity.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An isolated polypeptide comprising an amino acid sequence selected from the group consisting of:
(a) an amino acid sequence comprising SEQ ID NO: 6, 9, 12, or 53;
(b) an amino acid sequence comprising at least 85% sequence identity to SEQ ID NO:6, 9, 12, or 53, wherein said polypeptide has cytokinin oxidase activity;
(c) an amino acid sequence encoded by a nucleotide sequence that hybridizes under stringent conditions to the complement of SEQ ID NO: 5, 8, 11, or 52, wherein said stringent conditions comprise hybridization in 50% formamide, 1 M NaCl, 1% SDS at 37° C., and a wash in 0.1×SSC at 60° C. to 65° C.; and,
(d) an amino acid sequence comprising at least 17 consecutive amino acids of SEQ ID NO: 6, 9, 12, or 53, wherein said polypeptide retains cytokinin oxidase activity.

2. An isolated polynucleotide comprising a nucleotide sequence selected from the group consisting of:
(a) a nucleotide sequence comprising SEQ ID NO: 4, 5, 7, 8, 10, 11, 51, or 52;
(b) a nucleotide sequence encoding an amino acid sequence comprising SEQ ID NO: 6, 9, 12, or 53;
(c) a nucleotide sequence comprising at least 85% sequence identity to SEQ ID NO: 4, 5, 7, 8, 10, 11, 51, or 52, or to the coding sequence thereof, wherein said polynucleotide encodes a polypeptide having cytokinin oxidase activity;
(d) a nucleotide sequence comprising at least 50 consecutive nucleotides of SEQ ID NO: 4, 5, 7, 8, 10, 11, 51, or 52, or a complement thereof; and,
(e) a nucleotide sequence that hybridizes under stringent conditions to the complement of the nucleotide sequence of a), wherein said stringent conditions comprise hybridization in 50% formamide, 1 M NaCl, 1% SDS at 37° C., and a wash in 0.1×SSC at 60° C. to 65° C.

3. An expression cassette comprising the polynucleotide of claim 2 operably linked to a promoter that drives expression in a plant.

4. A plant comprising the expression cassette of claim 3.

5. The plant of claim 4, wherein said plant has a modulated cytokinin level when compared to a control plant.

6. The plant of claim 5, wherein said cytokinin level is increased when compared to a control plant.

7. The plant of claim 5, wherein said cytokinin level is decreased when compared to a control plant.

8. The expression cassette of claim 3, wherein said promoter is a tissue-specific promoter, a constitutive promoter, or an inducible promoter.

9. The plant of claim 4, wherein said plant has modulated floral development when compared to a control plant.

10. The plant of claim 4, wherein said plant has modulated root development when compared to a control plant.

11. The plant of claim 4, wherein the plant has an altered shoot-to-root ratio when compared to a control plant.

12. The plant of claim 4, wherein said plant has an increased seed size or an increased seed weight, or both, when compared to a control plant.

13. The plant of claim 4, wherein plant yield or plant vigor of said plant is increased when compared to a control plant.

14. The plant of claim 4, wherein the stress tolerance of said plant is maintained or improved when compared to a control plant.

15. The plant of claim 14, wherein said plant is Zea mays and tip kernel abortion is reduced.

16. The plant of claim 14, wherein the seed set of said plant under stress is increased or maintained when compared to a control plant.

17. The plant of claim 4, wherein said plant has a decrease in shoot growth when compared to a control plant.

18. A transformed seed of the plant of claim 4.

19. A plant that is genetically modified at a native genomic locus, said genomic locus encoding a polypeptide selected from the group consisting of:
(a) an amino acid sequence comprising SEQ ID NO: 3, 6, 9, 12, or 53; and
(b) an amino acid sequence comprising at least 85% sequence identity to SEQ ID NO: 3, 6, 9, 12, or 53, wherein said polypeptide has cytokinin oxidase activity;
wherein said plant is genetically modified to reduce or eliminate the activity of said polypeptide.

20. The plant of claim 19, wherein activity of said polypeptide is reduced or eliminated by means of a Mu insertion in the genomic locus.

21. The plant of claim 19, wherein activity of said polypeptide is reduced or eliminated by means of a hairpin construct specific to the genomic locus.

22. A method for increasing the level or activity of a cytokinin oxidase polypeptide in a plant, comprising introducing into said plant a polynucleotide comprising a nucleotide sequence selected from the group consisting of:
(a) a nucleotide sequence comprising SEQ ID NO: 2, 5, 8, 11, 52, 54, or 55;
(b) a nucleotide sequence encoding an amino acid sequence comprising SEQ ID NO: 3, 6, 9,12, or 53;
(c) a nucleotide sequence having at least 85% sequence identity to SEQ ID NO: 2, 5, 8, 11, 52, 54, or 55, wherein said polynucleotide encodes a polypeptide having cytokinin oxidase activity;
(d) a nucleotide sequence that hybridizes under stringent conditions to the complement of the polynucleotide of a), wherein said stringent conditions comprise hybridization in 50% formamide, 1 M NaCl, 1% SDS at 37° C., and a wash in 0.1×SSC at 60° C. to 65° C., and wherein said polynucleotide encodes a polypeptide having cytokinin oxidase activity; and,
(e) a nucleotide sequence comprising at least 50 consecutive nucleotides of SEQ ID NO: 2, 5, 8, 11, 52, 54, or 55, wherein said polynucleotide encodes a polypeptide having cytokinin oxidase activity.

23. The method of claim 22, wherein providing said polypeptide decreases the level or activity of cytokinin in said plant.

24. The method of claim 22, wherein said polynucleotide is operably linked to a tissue-specific promoter, a constitutive promoter, or an inducible promoter.

25. The method of claim 22, wherein increasing the activity of said polypeptide modulates root development of the plant compared to a control plant.

26. The method of claim 22, wherein increasing the activity of the polypeptide modulates floral development.

27. A method for reducing or eliminating the level of a polypeptide in a plant comprising modifying a native genomic locus encoding said polypeptide, wherein said native genomic locus comprises a nucleotide sequence selected from the group consisting of:
(a) SEQ ID NO: 1, 2, 4, 5, 7, 8, 10, 11, 51, 52, 54, or 55;
(b) a nucleotide sequence encoding an amino acid sequence comprising SEQ ID NO: 3, 6, 9, 12, or 53;
(c) a nucleotide sequence comprising at least 85% sequence identity to SEQ ID NO: 2, 5, 8, 11, 52, 54, or 55, wherein said polynucleotide encodes a polypeptide having cytokinin oxidase activity;
(d) a nucleotide sequence comprising at least 50 consecutive nucleotides of SEQ ID NO: 1, 2, 4, 5, 7, 8, 10, 11, 51, 52, 54, or 55, or a complement thereof; and,
(e) a nucleotide sequence that hybridizes under stringent conditions to the complement of the nucleotide sequence of a), wherein said stringent conditions comprise hybridization in 50% formamide, 1 M NaCl, 1% SDS at 37° C., and a wash in 0.1×SSC at 60° C. to 65° C.

28. The method of claim 27, wherein said method increases the level of cytokinin in the plant.

29. The method of claim 27, wherein reducing the level of said polypeptide increases seed size or seed weight of the plant when compared to a control plant.

30. The method of claim 27, wherein said plant is maize, wheat, rice, barley, sorghum, or rye.

31. The method of claim 27, wherein the stress tolerance of said plant is maintained or improved when compared to a control plant.

32. The method of claim 31, wherein the plant is Zea mays and tip kernel abortion is minimized.

33. The method of claim 27, wherein said native genomic locus is modified by means of a Mu insertion.

34. The method of claim 27, wherein said native genomic locus is modified by means of a hairpin construct.

35. A first polynucleotide comprising a nucleotide sequence comprising SEQ ID NO: 13, 14, 15, or 16, or nucleotides 1-3390 of SEQ ID NO: 51, wherein said polynucleotide drives expression of a second, operably linked, polynucleotide.

36. A DNA construct comprising a promoter operably linked to a nucleotide sequence of interest, wherein said promoter comprises the polynucleotide of claim 35 or a functional fragment thereof.

37. A method of regulating the expression of a nucleotide sequence of interest, said method comprising stably incorporating in the genome of a plant cell a nucleotide sequence of interest operably linked to a promoter comprising the nucleotide sequence of claim 36.

38. A construct comprising a first nucleotide sequence which shares sufficient sequence identity with SEQ ID NO: 13, 14, 15, 16, or 51 that, when expressed, a hairpin RNA molecule is formed which suppresses expression of a second nucleotide sequence operably linked to the ZmCkx2 promoter, ZmCkx3 promoter, ZmCkx4 promoter, ZmCkx5 promoter, or ZmCkx6 promoter, respectively.

39. A method of suppressing cytokinin oxidase activity in a plant, comprising transformation of a plant host cell with the genetic construct of claim 38 and regenerating from said transformed cell a transgenic plant wherein expression of one or more endogenous cytokinin oxidase gene is reduced or eliminated.


SEQUENCE LISTING (56 sequences)

SEQ ID NO: 1 - DNA, 3200 bp, Zea mays
  exon (267)...(849)
  exon (957)...(1084)
  exon (1175)...(1435)
  exon (1509)...(1771)
  exon (1871)...(2195)
  polyA_signal (2859)...(2864)
  misc_feature (0)...(0): Genomic sequence for ZmCkx2
cctatatagagaggccccctccctccccctgcatggacagccaccgccttcttcaaccctccttccgtcttcctcctctagtcttacctcgttgcacctcaagaaacttggcgcgcaaccaggaaaccccctcttctctctctctctctctctctctctctgccttctgattccaagctccccaactgcccagcaccaacctgccgaactcccctcctttttgttggtttgtcgaattataaattgagcccggccggctgactaccatgaagccgccatcactggtgcactgcttcaagctgctggtcctgctggcgctcgccaggctgaccatgcacgtccccgacgaggacatgctatcgcccctcggcgcgctgcgcctcgacggtcatttcagcttccatgacgtctccgccatggcgcgggacttcggcaaccagtgcagcttcctgccggccgccgtgctccacccaggctcggtctccgatatcgccgccaccgtgaggcacgtcttctccctgggcgagggctcgccgctcaccgtcgcggcgcgcgggcatggacactccctcatgggtcagtcccaggccgcccaggggatcgtggtcaggatggagtcgctccggggcgctaggctccaggtccacgacggctttgtcgatgcccccggaggagagctctggatcaatgtcctgcgtgagacgctgaagcacggcctggcacccaagtcgtggacggactatctccatctcacggtcggtggcaccttgtctaatgcgggggtcagcggccaggcgttccgccacggaccgcaggtcagcaatgtcaatcaactggagattgtgacaggtctcaaacgaactcacaaagcattcaatcaactagcttggagcatacataacgaaacataaaaaaaaacagtcgctgatcgtaataatcgtaaaaaccaaatgcaggaaggggagacgtcgttacctgctcacccgaggataactctgatctcttctatgctgctctcggcggtcttggtcagttcgggatcataaccagagcaaggattgcacttgagcctgctccagagatggtaagtcatcagacaagcgattcagttaaatgaaatctccagacagcatgcagtcatttagtaaatggatgtatatatatacaatgacaggtgaggtggataagagttctttactcggattttgaaagcttcaccgaagaccaggagatgttgatcatggcagagaactcctttgactacattgaaggttttgtcatcataaacaggacaggcatcctcaacaactggagggcgtccttcaagccacaggacccagtccaagcaagccatttccagtcagatggaagagtgctatactgcctcgaactaaccaagaacttcaatagtggcgacactgataccatggaacaggtgagcctgttatttcactttgcaccaagatattagactccaatgataataactgtaaattttatgtttacaggaagttgctgtactgctatctcggcttagattcatacagtctactctattccacaccgatgtcacgtacctggagtttttggacagggtgcacacctctgagctgaagctgagggcacaaagcctctgggaagttccacacccttggttgaatcttctgataccgaggagctcaatccgcagatttgctacggaagtctttggcaggatcctgaaagatagcaacaatggtcctatattgctttatccagtgaacaaatcaaagtaacttccttcacttgcaaaaattactgtcacaaataataagttaatctagttgcgcacggttaaggtagctcaattcgtctgttcgttctgatgcaggtgggacaacaaaacgtcagtggtcataccagatgaggaaattttctacctagtgggattcctttcttcagcaccgtctctctcaggtcacggcagcattgcacatgcgatgagcctgaacagccaaatagtagagttctgtgaagaggctgatattgggatgaaacagtatctagcacactacaccacacaggagcagtggaaaacccactttggagcaaggtgggagacatttgaacggaggaaacacagatatgatcccctagccatcctagcaccaggacagagaatattcccaaaggcgtcactcccattgtctttgtgacggttcctgctatttaaaggcttctgtagagcatacattgtacaaaagtgtaggtaaaagtatcccctgtaaagacaatatctacggaaggtagctagcctgaagaacacagcatagcgactttttcagtggccaaagatacctcaaagcagtacttcaatgtggagcaacgtcacctgaaccctgaaggtggtgagtgcaactttggaggcaatcactggtagtggagcctggagggttgtagcggtccaaggaacctgtctgttgttacagcgttgagatgagctgtgctgatcaactgatcactaaccagtgtcccgaggaaatcatgttggtctgtatgtattttccgttaacaacagtgcagaagtttgcatgagggtagtgcattgattagcaaatagcactgcctgttatttcacttgtaactggcatctcatctcaaggagagcctgcgtaactgtagcaggttatattgttttccatgagtcagaaactcagaatattaatgctcgtgcaaaaacagtgtagtcgcttatcaatcatggtgttcagaaacagaaaaactcttggaattctctcaacttgttcattaaatgttagcaacctatagtgtgagcttggataacaaataagaaatcaagagcgcaaatattgaaactgaataaatgttaaatgataattttctaaagtccaatcaagcagaattataagtttgcaagataacttgataactgacatctcagttttttgtatcaagcaaatgtgctgtcaaaaacaaaagcaaatacacccttttcagttagtgggccatactgtggtcttaatcagacctttgtttccgcaataaggtgttcatggaactgcatgtgcgatacagcttctgctcatgatacaaccaacataagatctcaatagagaagtattcatatcccgtaacagcgcaatgttcaaagatatttgcctggttcaaatacgggcatgcagttcttcacgatcgtggta

SEQ ID NO: 2 - DNA, 1560 bp, Zea mays
  CDS (1)...(1560)
  misc_feature (0)...(0): ZmCkx2 cDNA
atgaagccgccatcactggtgcactgcttcaagctgctggtcctgctggcgctcgccaggctgaccatgcacgtccccgacgaggacatgctatcgcccctcggcgcgctgcgcctcgacggtcatttcagcttccatgacgtctccgccatggcgcgggacttcggcaaccagtgcagcttcctgccggccgccgtgctccacccaggctcggtctccgatatcgccgccaccgtgaggcacgtcttctccctgggcgagggctcgccgctcaccgtcgcggcgcgcgggcatggacactccctcatgggtcagtcccaggccgcccaggggatcgtggtcaggatggagtcgctccggggcgctaggctccaggtccacgacggctttgtcgatgcccccggaggagagctctggatcaatgtcctgcgtgagacgctgaagcacggcctggcacccaagtcgtggacggactatctccatctcacggtcggtggcaccttgtctaatgcgggggtcagcggccaggcgttccgccacggaccgcaggtcagcaatgtcaatcaactggagattgtgacaggaaggggagacgtcgttacctgctcacccgaggataactctgatctcttctatgctgctctcggcggtcttggtcagttcgggatcataaccagagcaaggattgcacttgagcctgctccagagatggtgaggtggataagagttctttactcggattttgaaagcttcaccgaagaccaggagatgttgatcatggcagagaactcctttgactacattgaaggttttgtcatcataaacaggacaggcatcctcaacaactggagggcgtccttcaagccacaggacccagtccaagcaagccatttccagtcagatggaagagtgctatactgcctcgaactaaccaagaacttcaatagtggcgacactgataccatggaacaggaagttgctgtactgctatctcggcttagattcatacagtctactctattccacaccgatgtcacgtacctggagtttttggacagggtgcacacctctgagctgaagctgagggcacaaagcctctgggaagttccacacccttggttgaatcttctgataccgaggagctcaatccgcagatttgctacggaagtctttggcaggatcctgaaagatagcaacaatggtcctatattgctttatccagtgaacaaatcaaagtgggacaacaaaacgtcagtggtcataccagatgaggaaattttctacctagtgggattcctttcttcagcaccgtctctctcaggtcacggcagcattgcacatgcgatgagcctgaacagccaaatagtagagttctgtgaagaggctgatattgggatgaaacagtatctagcacactacaccacacaggagcagtggaaaacccactttggagcaaggtgggagacatttgaacggaggaaacacagatatgatcccctagccatcctagcaccaggacagagaatattcccaaaggcgtcactcccattgtctttgtga

SEQ ID NO: 3 - protein, 519 aa, Zea mays
MKPPSLVHCFKLLVLLALARLTMHVPDEDMLSPLGALRLDGHFSFHDVSAMARDFGNQCSFLPAAVLHPGSVSDIAATVRHVFSLGEGSPLTVAARGHGHSLMGQSQAAQGIVVRMESLRGARLQVHDGFVDAPGGELWINVLRETLKHGLAPKSWTDYLHLTVGGTLSNAGVSGQAFRHGPQVSNVNQLEIVTGRGDVVTCSPEDNSDLFYAALGGLGQFGIITRARIALEPAPEMVRWIRVLYSDFESFTEDQEMLIMAENSFDYIEGFVIINRTGILNNWRASFKPQDPVQASHFQSDGRVLYCLELTKNFNSGDTDTMEQEVAVLLSRLRFIQSTLFHTDVTYLEFLDRVHTSELKLRAQSLWEVPHPWLNLLIPRSSIRRFATEVFGRILKDSNNGPILLYPVNKSKWDNKTSVVIPDEEIFYLVGFLSSAPSLSGHGSIAHAMSLNSQIVEFCEEADIGMKQYLAHYTTQEQWKTHFGARWETFERRKHRYDPLAILAPGQRIFPKASLPLSL

SEQ ID NO: 4 - DNA, 3258 bp, Zea mays
  misc_feature (0)...(0): Genomic sequence for ZmCkx3
aaaaaatgttntncagatatatgtataaaaatgtgtacctagtacctacgcatgtcttagttcaacatacttgatagctgtagttttctgaaacctgttcaaattaacctttttcctacctgatggtgaatagagagaaaagctttacctttgtctgaataagaaaactaacagaaagcttacattttggccactctacctgcccgagtattttctaagcaagcaaaggcgcatgaaaattttctcggaatccatgaccttttacgcgcatgaaaattttgaccaatgatcattttgatactctccacaagtcaacatctcaaaaccacaagatggggcccatcaacataagttcacgagtgtgccttcaggtacattgttctttttttttgttttgctaaagtcaatcagctgcaaaatattcagaacaatttcaataacccgaaaggctgttgtgcctccatttgtcaacgtttgcgaggccaaatggtacccccgctataaataccatggaagttcttggcctctaggacacacaagcgatctctcctcctatagtttctataaccccacaaagcgtccaggtcccgtagtcacctccgattgcattgcgttgccgcaagacaagcatggcaagaaggactcgtttcgtggccatcgccgccctcctcacaagcttcctcaacgtcgcagccgggcattcccggccactgtccggtgccggcctcccgggcgatcttttcgggctgggcatcgcgtcgaggatccgcacggacagcaactcgacggcgaaggcggcgacggacttcggccagatggtgagggccgcgccggaggccgtgttccaccccgccacgccggccgacatcgccgcgctcgtccggttctccgccacgtcggcggcgccgttccccgttgcgccgcgcgggcagggccactcctggcgcggccaggcgctcgccccgggcggcgtcgtcgtggacatgggctcgctggggcgcggcccccgcatcaacgtgtccgccgtggccggcgcggagccgttcgtcgacgccggcggggagcagctgtgggtcgacgtcctccgcgccacgctgcgacacggcctggcgccccgcgtgtggaccgactacctccggctcaccgtcggcggcacgctctccaacgcgggaatcggcgggcaggcgttccgacacggtccgcagatcgccaacgtgcatgaactcgacgtcgtcacaggtatcgaccgatcgatggttacactcccagtgacaattacataagcagctaatcacacacgaatgctaataatagtttatacatgcgatgaaaaatgtaggcacaggtgagatggtgacatgctccatggacgtgaactcggacctgttcatggcggctctaggcgggttaggccagttcggggtcataaccagagcacggatccggcttgagccggcgcccaagagggtgcgctgggttcgacttgcctacaccgacgtcgctactttcaccaaggatcaggagtttctcatatcaaaccgggctagccaagtcgggttcgactacgtcgaaggccaggtccagctcagccggtccttggtcgaaggccccaaatcaacacccttcttctccggcgccgatgttgctaggcttgctggactcgcgtccaggaccggacctgctgcaatctactacatcgaaggcgccatgtactacaccaaggacaccgccatatctgtggacaaggtacagatcagcttgaacacacacacaaaaaaacgaactttattattgctttcaatgctttggacgaaaggaaattcattcgttgttgctatatgaaacgttgcagaaaatgaaggcactcctggatcagctgagcttcgagccagggtttgcgttcaccaaggacgtgacgttcgtgcagttcctcgatcgggtgcgcgaggaggagagggtgctccggtcagccggcgcgtgggaggtgccgcacccatggctgaacctcttcgtcccacggtcgcgcatcctcgacttcgacgacggagtgttcaaggctctgctcaaggactccaacccagctgggatcatcctcatgtaccccatgaacaaggataggtgggacgaccggatgacagcgatgaccccagccacggacgacgacgacatgttctatgccgttagtttcctttggtcagcactgtccgcagacgacgtgccccagctcgagagatggaacaaggcagtgctggacttctgtgatcggtcaggaatagaatgcaagcagtacctgccacactacacatctcaagacgggtggcgacggcatttcggggcgaaatggagcaggatcgctgagctgaaggccagatatgaccctcgggcattgttgtcgccgggccagaggatttttccggtgccagtagaggcatctggcattgcttctgcctgattgcccggtctcgtagtctcgaagcaaacataaatgattttcttgtgtagattgtagaatgtacatgataggtctttttcattgtaagaaagataggtcttattttgtacataatttttcttttgggttgctagcacggacggaggggccattgtggctagagaaaggtaataatagtcacaaattatataattcacatctcaccttttaagtattaagaagtcatttaaaaagaaggatagacaaatgcaattgccttagtctctaagatttatatagcaaaattatcagtataactttgtattgtgtattatttaccccgcatatgtttcttaacagtttattcttattttagacaatattctattttatacaatttttttacagtagaatcccacggtacactcgaaactaggatggggctccaaatcggagcaggttttaatataagagatgggaaagaagaaccagattaatactaccctctcctatcaaataaatttgcattccattaaaaaaattgaaaaactgaaaaaaaatcagtacaagtagagccaagatttgaatttggcaaatacttataaaacattttgaggcattgatatgttagctagaggccgagagccaagtatgtcggatggtaaatcgagaagcgggcagtttgctaaaatatctttgttttatttttgtcatttaaactagggatgacaatggggatttttccgtcggggaatggctccccatccccgtcctcgtggggtggaaaattcctcgtccccgtccccgcgaacacccacgggaagctt

SEQ ID NO: 5 - DNA, 2635 bp, Zea mays
  misc_feature (0)...(0): ZmCkx3 cDNA
aaaaaatgttntncagatatatgtataaaaatgtgtacctagtacctacgcatgtcttagttcaacatacttgatagctgtagttttctgaaacctgttcaaattaacctttttcctacctgatggtgaatagagagaaaagctttacctttgtctgaataagaaaactaacagaaagcttacattttggccactctacctgcccgagtattttctaagcaagcaaaggcgcatgaaaattttctcggaatccatgaccttttacgcgcatgaaaattttgaccaatgatcattttgatactctccacaagtcaacatctcaaaaccacaagatggggcccatcaacataagttcacgagtgtgccttcaggtacattgttctttttttttgttttgctaaagtcaatcagctgcaaaatattcagaacaatttcaataacccgaaaggctgttgtgcctccatttgtcaacgtttgcgaggccaaatggtacccccgctataaataccatggaagttcttggcctctaggacacacaagcgatctctcctcctatagtttctataaccccacaaagcgtccaggtcccgtagtcacctccgattgcattgcgttgccgcaagacaagcatggcaagaaggactcgtttcgtggccatcgccgccctcctcacaagcttcctcaacgtcgcagccgggcattcccggccactgtccggtgccggcctcccgggcgatcttttcgggctgggcatcgcgtcgaggatccgcacggacagcaactcgacggcgaaggcggcgacggacttcggccagatggtgagggccgcgccggaggccgtgttccaccccgccacgccggccgacatcgccgcgctcgtccggttctccgccacgtcggcggcgccgttccccgttgcgccgcgcgggcagggccactcctggcgcggccaggcgctcgccccgggcggcgtcgtcgtggacatgggctcgctggggcgcggcccccgcatcaacgtgtccgccgtggccggcgcggagccgttcgtcgacgccggcggggagcagctgtgggtcgacgtcctccgcgccacgctgcgacacggcctggcgccccgcgtgtggaccgactacctccggctcaccgtcggcggcacgctctccaacgcgggaatcggcgggcaggcgttccgacacggtccgcagatcgccaacgtgcatgaactcgacgtcgtcacaggcacaggtgagatggtgacatgctccatggacgtgaactcggacctgttcatggcggctctaggcgggttaggccagttcggggtcataaccagagcacggatccggcttgagccggcgcccaagagggtgcgctgggttcgacttgcctacaccgacgtcgctactttcaccaaggatcaggagtttctcatatcaaaccgggctagccaagtcgggttcgactacgtcgaaggccaggtccagctcagccggtccttggtcgaaggccccaaatcaacacccttcttctccggcgccgatgttgctaggcttgctggactcgcgtccaggaccggacctgctgcaatctactacatcgaaggcgccatgtactacaccaaggacaccgccatatctgtggacaagaaaatgaaggcactcctggatcagctgagcttcgagccagggtttgcgttcaccaaggacgtgacgttcgtgcagttcctcgatcgggtgcgcgaggaggagagggtgctccggtcagccggcgcgtgggaggtgccgcacccatggctgaacctcttcgtcccacggtcgcgcatcctcgacttcgacgacggagtgttcaaggctctgctcaaggactccaacccagctgggatcatcctcatgtaccccatgaacaaggataggtgggacgaccggatgacagcgatgaccccagccacggacgacgacgacatgttctatgccgttagtttcctttggtcagcactgtccgcagacgacgtgccccagctcgagagatggaacaaggcagtgctggacttctgtgatcggtcaggaatagaatgcaagcagtacctgccacactacacatctcaagacgggtggcgacggcatttcggggcgaaatggagcaggatcgctgagctgaaggccagatatgaccctcgggcattgttgtcgccgggccagaggatttttccggtgccagtagaggcatctggcattgcttctgcctgattgcccggtctcgtagtctcgaagcaaacataaatgattttcttgtgtagattgtagaatgtacatgataggtctttttcattgtaagaaagataggtcttattttgtacataatttttcttttgggttgctagcacggacggaggggccattgtggctagagaaaggtaataatagtcacaaattatataattcacatctcaccttttaagtattaagaagtcatttaaaaagaaggatagacaaatgcaattgccttagtctctaagatttatatagcaaaattatcagtataactttgtattgtgtattatttaccccgcatatgtttcttaacagtttattcttattttagacaatattctattttatacaatttttttacagta

SEQ ID NO: 6 - protein, 538 aa, Zea mays
MARRTRFVAIAALLTSFLNVAAGHSRPLSGAGLPGDLFGLGIASRIRTDSNSTAKAATDFGQMVRAAPEAVFHPATPADIAALVRFSATSAAPFPVAPRGQGHSWRGQALAPGGVVVDMGSLGRGPRINVSAVAGAEPFVDAGGEQLWVDVLRATLRHGLAPRVWTDYLRLTVGGTLSNAGIGGQAFRHGPQIANVHELDVVTGTGEMVTCSMDVNSDLFMAALGGLGQFGVITRARIRLEPAPKRVRWVRLAYTDVATFTKDQEFLISNRASQVGFDYVEGQVQLSRSLVEGPKSTPFFSGADVARLAGLASRTGPAAIYYIEGAMYYTKDTAISVDKKMKALLDQLSFEPGFAFTKDVTFVQFLDRVREEERVLRSAGAWEVPHPWLNLFVPRSRILDFDDGVFKALLKDSNPAGIILMYPMNKDRWDDRMTAMTPATDDDDMFYAVSFLWSALSADDVPQLERWNKAVLDFCDRSGIECKQYLPHYTSQDGWRRHFGAKWSRIAELKARYDPRALLSPGQRIFPVPVEASGIASA

SEQ ID NO: 7 - DNA, 6177 bp, Zea mays
  misc_feature (0)...(0): Genomic sequence for ZmCkx4
ctttatgttgtagccaaggaaagtatactgttaagatcagaatgaaccttataggagttgtatgggcataaagccagcaagtatagccaaaggtacacaaggctaatatagtcaagttgttgatgtgtgagacgttcaaggaagtgaactattggaggagtcgactaaaagtacgattaataaggtagacatgatggtaaaatctttgatctagaatttaagtggtatggatgcgagggtgagaatggcaagcacaacttcaaatatagggtgatgcttatgcttggctgagccatttcattcatgagcataggaacatgagacatggtgggatatggatacttgcacaaaaaaaggaattaagtttatgatattcacctcccagtcagtttgcatggtaaaaaaattcctatcaatttggttctcaactagggcctaaaattctcaaaatatctgttggggaccattatcgtcgacgatcctcagaatctgttattaccaaattaaaaggtgtgtttcaggtactgtgcaaagcagcagcgaagctatccttcgtcaaaagtggctcaatgaaccaggtggagaagctatggagcttcgtctgcgtagagcgtgccggaggaggaagctttggctctgaatgcatcgacttacgaagcatgggagaagaagactcagaaggcttgtccagcgtgggaataaaaaggagaaaatacaattttgcccttgtgggatttgtaaatcatgtgcaaggctcatggatatgtttgtaattttatatgatatgtttgtaaatcatggatatgttttgtaaatcaggtggactagaggagagggagggtggacatagtgacttgcatcttgatcatggtagagtggtcatggtagagggaaaggggtaggtcaattctggagtgcggccacggtggcttgagtgtcggccacggtaggggaaaggggtagcccaattctagggccggcatcggagaaggccgacatgtgcacgtcaggaggtagtgttagaggtttgaacggaaaaaattgaacatgttagtatgatgagttgtgtaattgctgggaattgtggataatttccacttaactacggccctgtttatttacccctagattataaaatccaacttaaaaaagttgagatgtaaacaaacaacacatattattaggtggattatgttatctagaaatctggatgataataatttataagtcggttaataggtgtttacataatcgataagctggattatataatcctggaacacggctttcgcgagagcgtattaaaacaggattccgtgaagcacactatctgaggagctccaccaaaagctgaatctagcccgcactcttttttggaggattcaaatttggtgtcactggagcattcggcattttgtttcatggcgtgaagctatttttactaattacagaagctgtttcaaatagacctttaaatgatggctgagtataaaaggaggcaatttttttatctcgccgatggagccaggtcgcgtcgcgccgcggccgtgctgcgctctcgacgcgatctagcggcgatgtgcacagtacagttttgccatgccattggttaagcctgcatacaacacaccagcgtactgccctgcacaagatctcctcggctcggcctctcctgatggaacgttcagcttgaacagcggagcgtgggggcatcccggggatgggcgccgcggccgagaaattttgcaacctggcaaatctgccctgtcgcatactaccatccacctccaggcgccaagaacgcctccgagtttcaggcttgcagctcagctctgtgttgaattggaacgggcggagtttctgggttccagacttccagtacaaggcgatcaattggtagggcgaattacttgcaggcccagatgcatggcccatctatctggttctctatcggttgcttttacttgcacaatagtggcagacaaactacaagtcagatccgatcctatccatccatccatctcgcagcgcgatgcaaatatgcaatcgtctgtggaactcgaaaaaaaacagaggtccggcctcgcacgaggttaagggaaaaaaaacgaagcgtttggaactttggttggcattcgcagcatgctgtgctgccaccgtatgtttttatttttgctttgtttgtcttctttgagaaacgtgagggagccgcgtgtccgctcgttataaaacccccccggcgacccaaactaccacgagctcaagcctcaagcctcaagcctcaagcaagcagagcgccgtgacatcacgaaacaaacatatagagctagctgctctgcctctgcttcaccaatcacctgcttggccgcgcggaggggagggtttccccctttgacacagctgagctcccctccatcagcagccagctcctcgtcgcaaagcaagaagatgatgctcgcgtacatggaccgcgcgacggcggccgccgagccagaggacgccggccgcgagcccgccaccatggcgggcgggtgcgcggcggcggcgacggatttcggcgggctggggagcgccatgcccgcggccgtggtccgcccggcgagcgcggacgacgtggccagcgccatccgcgcggcggcgctgacgccgcacctcaccgtggccgcccgcgggaacgggcactcggtggccggccaggccatggccgagggcgggctggtcctcgacatgcgctcgctcgcggcgccgtcccggcgcgcgcagatgcagctcgtcgtgcagtgccccgacggcggcggcggccgccgctgcttcgccgacgtccccggcggcgcgctctgggaggaggtgctccactgggccgtcgacaaccacgggctcgccccggcgtcctggacggactacctccgcctcaccgtgggcggcacgctctccaatggcggcgtcagcggccagtccttccgctacgggccccaggtgtccaacgtggccgagctcgaggtggtcaccggcgacggcgagcgccgcgtctgctcgccctcctcccacccggacctcttcttcgccgtgctcggcgggctcggccagtttggcgtcatcacgcgcgcccgcatcccgctccacagggcgcccaaggcggtgagcgcgcggacatcgggggcgaaagctaaagcttgctttttgcttgggcactactaactgactgacgttgccattcaggtgcggtggacgcgcgtggtgtacgcgagcatcgcggactacacggcggacgcggagtggctggtgacgcggccccccgacgcggcgttcgactacgtggagggcttcgcgttcgtgaacagcgacgaccccgtgaacggctggccgtccgtgcccatccccggcggcgcccgcttcgacccgtccctcctccccgccggcgccggccccgtcctctactgcctggaggtggccctgtaccagtacgcgcaccggcccgacgacgacgacgaggaggaccaggtaggtagcagtaattgccaacctctccccccgcttggcgcattcccgtacttgaccccctcgcccgctctggcgtgtacttttccgcgggcagggcatgtctgactcgcctcgtcgtgtatctcccgctggattcggtgacgggggtgctgcgtcctgccaaaccaaaccaccctagactagacagacccccaggggcaggggtcgcgccattggccgcacgcggggaccggcgccagtgagtgcgccgcgccgcacggccgcgccccgatctcgctcgctcgctcgctggtgatcgaatcggcgcgtacaatgcggcatggccccgagccccacacccgcagtggccgtgacgcgattgcgctgcctccggtccggcccatgacccagcggatcgcgtcgcgtcttttggcaacgcccgcgtcatcatatcgcgctctttgtcgtccccacggagcacagcgcagcgcagcgcagcgcagccaaccttttctccgccacgcacgcttcggcggcattcattatttggattttgttcctaccggtcgatccgcgtccgtccgtgcactgcaggcggctaccgtcatgctgaccaacccattgccattggttttgtttcttctctctctctccctctcgttggttatggttcgtgcgtgcctgcaggcggcggtgaccgtgagccggatgatggcgccgctcaagcacgtgcggggcctggagttcgcggcggacgtcgggtacgtggacttcctgtcccgcgtgaaccgggtggaggaggaggcccggcgcaacggcagctgggacgcgccgcacccgtggctcaacctcttcgtctccgcgcgcgacatcgccgacttcgaccgcgccgtcatcaagggcatgctcgccgacggcatcgacgggcccatgctcgtctaccctatgctcaagagcaagtgagttgccctccgctccgctccttcgcactgcgtgcagtagtacagtacaggagtggctgagtggtggtactgccattcagtgtgcagttgccgtttgcggcccgccaagctagctaggggccgggacgcatgtgagccgccctgccttctctctgctcgtcgtgtcactgacgcctggtcctccgggacagttgctgagccggcccgtacgtacctgtaagacgacggtcccgagcctccaccgccgcttctgttttggatttagccgtgtcacacagatcttacggaggaggaggagtactatgattgacaaattattgcttcgcccgacccgaggctagcgcacagtccatgtcatgtgggcctggctgtgtggtttccgtcctgatgctgatgcctgaagggaactgcgtgcgtgtgcgtgcgtgcaggtgggaccccaacacgtcggtggcgctgccggagggcgaggtcttctacctggtggcgctgctgcggttctgccggagcggcgggccggcggtggacgagctggtggcgcagaacggcgccatcctccgcgcctgccgcgccaacggctacgactacaaggcctacttcccgagctaccgcggcgaggccgactgggcgcgccacttcggcgccgccaggtggaggcgcttcgtggaccgcaaggcccggtacgacccgctggcgatcctcgcgccgggccagaagatcttccctcgggtcccggcgtccgtcgccgtgtagagcaaggggggaggaccagccagctgccagccaagacaggaggaggaggaggggaggctgatggatcgccgctgctgttgccggtaatgatggcgattacgctgctgatcctggtgatgatgatggacgatcgaggaagccgcagggccgggcaatgatggcgatagggccaccgttaggtgtgcatccgggggcacaaattaaagggattgctgtgtggagatctgcacgagtttttgctccatgcatgcttgccgttcgtgtccgcgtgtccctctcccccttgttattattccttcgcccgccgaggccgagcgagcgggtggtggcgacgctggatttgtctgctctgctctgctccgccgccgtggccaccccggtggcgtgcgcccgcaagctgttccttccgcgcgcttctgttccgttcggttcccccgtggtagcttccccccctcgccgtcctggtccccccgcccgcccggcaccccacgtggcacaccagcccgatccaaacgccgcgaccgcgacgcgcggggccgttggttcgcgttcccgttccgtagcagcttgcccgcagcacacgacgaccgcgaacaaagcgcggccaaaaccgacgggtctcgccgccgccgccgcggacgcgcccacgggacaggaggaatatcactctggggccatccgcgcgggaccatagaactggtcgggtcgatgtcgatcgatatcggcactctgtgctggctggcgacgcggaccgagcggcagggacgtgacggttgctgccgcccgagcgcgacggcgaccgtccttcgtctctggggcggggcggcgttttcgtttggaaaatttgtggacttctacttgtatataaaaaaacacgatcggcgcacgtatacaaccagtcttcctttccctgtcgtgcccagtcgcattccgtgatgcgagccggatcgcgacggaagcggctcaacgagcgtcgtccctg

SEQ ID NO: 8 - DNA, 1816 bp, Zea mays
  misc_feature (0)...(0): cDNA for ZmCkx4
atgatgctcgcgtacatggaccgcgcgacggcggccgccgagccagaggacgccggccgcgagcccgccaccatggcgggcgggtgcgcggcggcggcgacggatttcggcgggctggggagcgccatgcccgcggccgtggtccgcccggcgagcgcggacgacgtggccagcgccatccgcgcggcggcgctgacgccgcacctcaccgtggccgcccgcgggaacgggcactcggtggccggccaggccatggccgagggcgggctggtcctcgacatgcgctcgctcgcggcgccgtcccggcgcgcgcagatgcagctcgtcgtgcagtgccccgacggcggcggcggccgccgctgcttcgccgacgtccccggcggcgcgctctgggaggaggtgctccactgggccgtcgacaaccacgggctcgccccggcgtcctggacggactacctccgcctcaccgtgggcggcacgctctccaatggcggcgtcagcggccagtccttccgctacgggccccaggtgtccaacgtggccgagctcgaggtggtcaccggcgacggcgagcgccgcgtctgctcgccctcctcccacccggacctcttcttcgccgtgctcggcgggctcggccagtttggcgtcatcacgcgcgcccgcatcccgctccacagggcgcccaaggcggtgcggtggacgcgcgtggtgtacgcgagcatcgcggactacacggcggacgcggagtggctggtgacgcggccccccgacgcggcgttcgactacgtggagggcttcgcgttcgtgaacagcgacgaccccgtgaacggctggccgtccgtgcccatccccggcggcgcccgcttcgacccgtccctcctccccgccggcgccggccccgtcctctactgcctggaggtggccctgtaccagtacgcgcaccggcccgacgacgacgacgaggaggaccaggcggcggtgaccgtgagccggatgatggcgccgctcaagcacgtgcggggcctggagttcgcggcggacgtcgggtacgtggacttcctgtcccgcgtgaaccgggtggaggaggaggcccggcgcaacggcagctgggacgcgccgcacccgtggctcaacctcttcgtctccgcgcgcgacatcgccgacttcgaccgcgccgtcatcaagggcatgctcgccgacggcatcgacgggcccatgctcgtctaccctatgctcaagagcaagtgggaccccaacacgtcggtggcgctgccggagggcgaggtcttctacctggtggcgctgctgcggttctgccggagcggcgggccggcggtggacgagctggtggcgcagaacggcgccatcctccgcgcctgccgcgccaacggctacgactacaaggcctacttcccgagctaccgcggcgaggccgactgggcgcgccacttcggcgccgccaggtggaggcgcttcgtggaccgcaaggcccggtacgacccgctggcgatcctcgcgccgggccagaagatcttccctcgggtcccggcgtccgtcgccgtgtagagcaaggggggaggaccagccagctgccagccaagacaggaggaggaggaggaggggaggctgatggatcgccgctgctgttgccggtaatgatggcgattacgctgctgatcctggtgatgatgatggacgatcgaggaagccgcagggccgggcaatgatggcgatagggccaccgttaggtgtgcatccgggggcgcaaattaaagggattgctgtgtggagatctgcacgagtttttgctccatgc

SEQ ID NO: 9 - protein, 521 aa, Zea mays
MMLAYMDRATAAAEPEDAGREPATMAGGCAAAATDFGGLGSAMPAAVVRPASADDVASAIRAAALTPHLTVAARGNGHSVAGQAMAEGGLVLDMRSLAAPSRRAQMQLVVQCPDGGGGRRCFADVPGGALWEEVLHWAVDNHGLAPASWTDYLRLTVGGTLSNGGVSGQSFRYGPQVSNVAELEVVTGDGERRVCSPSSHPDLFFAVLGGLGQFGVITRARIPLHRAPKAVRWTRVVYASIADYTADAEWLVTRPPDAAFDYVEGFAFVNSDDPVNGWPSVPIPGGARFDPSLLPAGAGPVLYCLEVALYQYAHRPDDDDEEDQAAVTVSRMMAPLKHVRGLEFAADVGYVDFLSRVNRVEEEARRNGSWDAPHPWLNLFVSARDIADFDRAVIKGMLADGIDGPMLVYPMLKSKWDPNTSVALPEGEVFYLVALLRFCRSGGPAVDELVAQNGAILRACRANGYDYKAYFPSYRGEADWARHFGAARWRRFVDRKARYDPLAILAPGQKIFPRVPASVAV

SEQ ID NO: 10 - DNA, 5108 bp, Zea mays
  misc_feature (0)...(0): Genomic sequence for ZmCkx5
atcccgaataaacaaatgaagtaggtcctcagtcacccttgccctgttagctgcaagagagctcatggtttccagccacacaatcagtccatggctccttcttcttggcctaagtggtggccaatcattgtgggtgatcgagtcttgggccctctgaacagtattacacaacagtaatcctgcaaaagatttggtatatctagattctagagtgagcgccgtgttgtgcccagctaggaatgggttgtcaagtgcaacaggaggaggacccaggatggtcaggtgtaataggctctcattaaaagactgttcagatggattagagcaacgacggggaagccgggaaaaaatggttggttctgctttcctctcgctccccggccgggttcatatatgaatctgagaacgatattttttgcttcatttttcatttgctatatatttaaactgtttttttgtgtgtgtgtgtgtgttcattgagctcaatacttgaggcttgatagggagaggagtgaggcagctgatcacatggacctccatctgaggacagttcctcttccgaaacagaaaggagagtgcagggaccagcgtggcctgtacagtattgtgtttgcccttttcctttggcagggacagagagcttcaggcttgtcctctttatgtatgctgctcgcctgcttcagagtcagagcttccccttctcacttctcagagagagagagagagaagagagagagaggagagccctccacagctcccctgtcctgccctcaggcattctttgtcacagggggcgagggctgaagatcatcacatggtggccttttttgggtctgtggcctttggtcttttagtgcttcttccttttacctcctcatgacatgaaccccctttttaaacctccctcaaaatcaaatcaccctccttctcctttaagagccctcaaccccttcccctcattttccttcatccctcagcctttgcacaaagggcaagaataacgcagtatgatcatctgatcatactcccgccgccatcacaatcccacacgaacgtgagacaaaggtaacagacgcaagaagctagcagctgcaggagattgctcagcccatctccatggaggttgccatggtcgtgagcgcaagagccagcctgctgatcctcgtcctctccctctgctctccgtacaaattcatacagagccccatggacctgggccccctgaacctgctccccaccaccagcaccgcggccgcgtccagcgacttcggcaggatactcttccgcgccccggccgcggtgctgaggccccagtcgccgagggacatctccatgctgctcagcttcctctccggctcgccctcgctgagcagggtcacggtggcggccaggggggcaggccactccatccacgggcaggcgcaggccccggacggcattgtggtggagacgcgctccttgcccggcgagatggagttccaccacgtccgcgggggaggcgaagggcgtgcctcctacgccgacgtgggcggcggggttctgtggatcgagctcctggagcggagcctgaagcttgggctggctcccaggtcctggaccgactacctctacctcactgtcggcgggacgctgtccaatgccggcatcagcgggcagacgttcaagcacgggccacagatcagcaacgtcctccagctggaggtagtcacaggtgagacacacgcacgcatgcatgcgtgcatgcatggtacatagatgaaacacaaagatcagatttttttttctgcctctctttcttgaccaacaaacaacctctctctctctctctctctctctctctctctctataacaaacaggacgaggggagattgtggaatgctcacccagcaaggaggccgacctgttcaatgccgtcctgggaggcctaggccagttcggcatcataaccagggccaggatcctgctgcaggaggctccggagaaggtgacgtgggtgagggccttctacgacgacttgggcgccttcaccagggaccaggagctgctggtgtcgattccggattcggtggactacgtggaagggttcatggtcctgaacgagcggtccctccacagctcctccatcgccttccccgcgagcgtggacttcagcccggatttcggcaccaggagcagccctaggatctactactgcgtcgagttcgcggtccaccaccaccacggttaccagcagcagtctcaggcggccgtggaggccatctcgaggcggatgagccacatggcgtcccagctgtacagcgtggaggtgtcctacttggacttcctgaaccgggtcaggatggaggaggtgagcctgcggagcgccgggatgtgggaggaggtgcaccacccgtggctcaacatgttcgtgcccaaggccggggtcgctggcttcagggatctgctcatggacaacgtctcgccggatagcttccagggcctcatcctcatctacccactcctcagagacaagtaagtaccactcttctaataataataataataatgatgatgacacaaaagctatatagtagtacatccatgaagatacgcctacactttcggcttttctcaaagcaaagcttatctgctttattaatcccggcctcttccggccgtcctcacttcatattagcttacaagaagtccaagttgggcaagcaagcatttctttgcattatccacagcaagttgcctccttgcgctgcctacagtggtaacgacatgggtagggtttggttttggagtaatagcgggatatgaagcctttccgctcttaatttgttgtttttagattgattttatataggacatagtgacacttaaaaaatatatgttcaaatattgaaccattttggtttcagaaaatctcttggaacgagccgttctagccgttctctctctagaacggagtcgctccatcctaactagcttcacaatcaaacattaccttggatgacgacgaagcgccgagaaaagtggtcactcatcgtgcgtgcattagggatgatagatcccttttgcttaattagcatgttggtttgttttttccctatgtccagcaaaggcgctcgtgggacccacgccgtcgtatagaggcgaatatgggttgttctatcgtgtgtttgtactagtggtccctcggatagcatcacatctgcgtcatcatcagcattgtatgaatcagctaaaactgtagatgagctcaatcagtcagtagcatcaaccttgagagtggcgacagggaaattataaaacatagtagtagctagctgatctgctttggaattagtcttcgtttttcttcttttttcacctaagggctagtttaggaacacaattttctcaaaaaaaaaattgaactaattactcttaagaaaatggaaattcctagaaaaaaaatgaggttgccaaactaggcttaaaagatttttttaacaggtgcaaacaacgtcgtaaacttgtaatcgatagcacaagttattcagttacaagcgtcttgttatgtgttcataccctccgaataaatttagacaagtttacatggatgcaaaaggggtttaaatatgtgtatgtgctagatcgcatcccagtgtcaactctgaactgattgagctttaacaaaagatgaagtcgtcacccaaagttccacgttcaccgcactggactagtgtatatataatctacaggcccaacagagacctctgacttgcctcaggaaaaaccactaaaccaaaagtctctctctctcgagagagagtcagaatacgctgaactgcagaatgtcagctacaccacccatacacaaattcctgctaccgcttatttgaaattcacagcgtctcgtcagcaagctctaaaaaactgtttgttgctgtcttcatcctttctttttttttatttgatttgattgacgacaggtgggacaccaacacgtcggtcgtgatcccggactccgggcccaccgcggacgacccggtgatgtacgtggtcggcatcctcaggtccgcgaaccctggtccagaagaagacggtgacggctgctcccaccgctgcctgcacgagctcctccgcagccaccgccggatcgccgacgccgcggaggcgcgcctcggcgccaagcagtacctgcctcaccacccgaccccggcccgctggcagcagcacctgggccggcgctgggagcgcttcgcggaccgcaaggcccggttcgacccgctgcgcatcctggggcccggccagggcatattccctcggacggcccaggatgctgccgccgctgctgcgtacgggagctagcattttatatatatacggcatgaaacagtatagattattaattatcagctgactagctaggatgcgatcttctttcttcttcttcttctctcttcagtttcctctgcattttgagggcatgtggggccggttgttgggttattctgtgaggctctggcctccggggcactttgagaggacctgatggtggtggtggtggtggtgattggtgattggtgaatgtcactgggaattttggaacttttgtacaggttgatggaggaagcacaggctttaagtttataagggaatagacatagcgcttctattcggtctctctattccttgttcgatatggccattgctagtgtcactattgtcctcggtaattccgtttctaggctgataaattccttccactttgtcagagccatcttctttaggagaagtggtgacaacgtgccagagccctttgggttagctcgagatcagaatgtagtccatggatgaggggccatgggtgctagatttacagtgaatttgatccgtccccaatgttccgttagctaaaatgcatgcggtgaagtcgtacaagcatgcatgatgcatgccctttttttctggagtacccgtcattttgccttctgaaactccggaagcccttgcattgaccgtttgacacaattatgaagtgaagatgataagtcgtgaatggattcatgacagatcaacacgtggcacctccgtacatacaaacacgcgcccgaagttccctctaggaaacttcaat

SEQ ID NO: 11 - DNA, 1629 bp, Zea mays
  misc_feature (0)...(0): cDNA for ZmCkx5
atggaggttgccatggtcgtgagcgcaagagccagcctgctgatcctcgtcctctccctctgctctccgtacaaattcatacagagccccatggacctgggccccctgaacctgctccccaccaccagcaccgcggccgcgtccagcgacttcggcaggatactcttccgcgccccggccgcggtgctgaggccccagtcgccgagggacatctccatgctgctcagcttcctctccggctcgccctcgctgagcagggtcacggtggcggccaggggggcaggccactccatccacgggcaggcgcaggccccggacggcattgtggtggagacgcgctccttgcccggcgagatggagttccaccacgtccgcgggggaggcgaagggcgtgcctcctacgccgacgtgggcggcggggttctgtggatcgagctcctggagcggagcctgaagcttgggctggctcccaggtcctggaccgactacctctacctcactgtcggcgggacgctgtccaatgccggcatcagcgggcagacgttcaagcacgggccacagatcagcaacgtcctccagctggaggtagtcacaggacgaggggagattgtggaatgctcacccagcaaggaggccgacctgttcaatgccgtcctgggaggcctaggccagttcggcatcataaccagggccaggatcctgctgcaggaggctccggagaaggtgacgtgggtgagggccttctacgacgacttgggcgccttcaccagggaccaggagctgctggtgtcgattccggattcggtggactacgtggaagggttcatggtcctgaacgagcggtccctccacagctcctccatcgccttccccgcgagcgtggacttcagcccggatttcggcaccaggagcagccctaggatctactactgcgtcgagttcgcggtccaccaccaccacggttaccagcagcagtctcaggcggccgtggaggccatctcgaggcggatgagccacatggcgtcccagctgtacagcgtggaggtgtcctacttggacttcctgaaccgggtcaggatggaggaggtgagcctgcggagcgccgggatgtgggaggaggtgcaccacccgtggctcaacatgttcgtgcccaaggccggggtcgctggcttcagggatctgctcatggacaacgtctcgccggatagcttccagggcctcatcctcatctacccactcctcagagacaagtgggacaccaacacgtcggtcgtgatcccggactccgggcccaccgcggacgacccggtgatgtacgtggtcggcatcctcaggtccgcgaaccctggtccagaagaagacggtgacggctgctcccaccgctgcctgcacgagctcctccgcagccaccgccggatcgccgacgccgcggaggcgcgcctcggcgccaagcagtacctgcctcaccacccgaccccggcccgctggcagcagcacctgggccggcgctgggagcgcttcgcggaccgcaaggcccggttcgacccgctgcgcatcctggggcccggccagggcatattccctcggacggcccaggatgctgccgccgctgctgcgtacgggagctag

SEQ ID NO: 12 - protein, 542 aa, Zea mays
MEVAMVVSARASLLILVLSLCSPYKFIQSPMDLGPLNLLPTTSTAAASSDFGRILFRAPAAVLRPQSPRDISMLLSFLSGSPSLSRVTVAARGAGHSIHGQAQAPDGIVVETRSLPGEMEFHHVRGGGEGRASYADVGGGVLWIELLERSLKLGLAPRSWTDYLYLTVGGTLSNAGISGQTFKHGPQISNVLQLEVVTGRGEIVECSPSKEADLFNAVLGGLGQFGIITRARILLQEAPEKVTWVRAFYDDLGAFTRDQELLVSIPDSVDYVEGFMVLNERSLHSSSIAFPASVDFSPDFGTRSSPRIYYCVEFAVHHHHGYQQQSQAAVEAISRRMSHMASQLYSVEVSYLDFLNRVRMEEVSLRSAGMWEEVHHPWLNMFVPKAGVAGFRDLLMDNVSPDSFQGLILIYPLLRDKWDTNTSVVIPDSGPTADDPVMYVVGILRSANPGPEEDGDGCSHRCLHELLRSHRRIADAAEARLGAKQYLPHHPTPARWQQHLGRRWERFADRKARFDPLRILGPGQGIFPRTAQDAAAAAAYGS

SEQ ID NO: 13 - DNA, 3003 bp, Zea mays
  misc_feature (0)...(0): ZmCkx2 promoter
ctgccatcctcatgcagatgagacggagagaagatgagaaaagtacaagatcccagaagcaagcagcaggatggggccatccccccccccccccactgggccccacgggccgaaagccaccggcgaaaatgtccagaaggccacgtggggcatgggtccccggagtccacttccgcgcgatctcgaggccgggccgcaccggcaatcgctctccggccacctccctgcttcctcaggtccggtctcccatagtccaatgcatgcatgcacgagcatcccctcagaacgctggcagtgagtgtcttgctcgcacatcagcttggccagtcagtgcgagaacacagcagcaacaacaacaacaacacctgtgcacaatggcgtctatcattggtaccatctcaatcggctgacttgtctataactactgttaacggaggtcccttgtgcatcatgcagttttagaagagcacctcgatcgcaagcgcctcattattatcatcattctcttaaactggtcagaaaactgaccatcagctaaagtgatactgacatactgtatctttgtagataattaaatggagaaaaatctccttctgttccgtctggccgttaaatgccgaatccatgcatatataaatctgtacgtaggctcaaagcacagtgtgcattttggctttccagctagcatacatacatgtgactgctgacgatgaattgtgtggaccacattggcacaacggtgcattgcaacggacgggcgccgtcaaggtcaaacgcataaaaggctgtcatttggcaacacaatgaatcagtggcgccacgccatccgtccacgatccaccgttcttggtgtagtggttggtcccagcgcttgaaggccaggccgaggccgtgttctggaaggtggcctgtggtgagcactaaacatgtgtgtgcttttgcctttccaagccagagggccggtctcttaatatacataacatacacaccactttttcattttgttcattattacggtctaatgcaaacaaagccatttgcagaatgtgctacatagcaggtatgtttctctttttttccctgtaaaatttgtagacttatcacaagaataagtttaaccattactagaatagttcctcacatgtttgtttaccatcggggcgggaacagcttgcattgcaaaagctgcgcaagtattagggccctctagatttttttaatagtagtagtatatataatatataggtgttactatttgagttgttaggccatctgcggcagattttctatgacatcccttatttcaaactttattttgcaaacagttgtcatataccctattttaggcgaatcactgaagacaggtaagttttggcacggatgaggtggagagtggacaagaatctccgttgtggagtctgcctaccagtaccaggcaaagtaatgcatgcgcgcggacaggatggacggtcgaagtggcctccctgcctccaccccgacgacgacgcatgggctccgtccccttcgcttgcttcctgctccagctagctccatcgcctagtgctccgctccgccgcacaggaacggaacggaacggaccgaaccacttggtcgcatcccgatgcgttgccgtctgccggtgtccatcgtgtcggtttcacctctgcactagcataaattccttgacaccaacagcgagcgacatcatcggctcagccctacaagtcacgagtgttctgactgaccagctagcaatagcaatctgctgctctgcttgacttgctcggacgatccgccgctgcttgcgttcggctccagtaggctatcctccgcgacgtcgtcgatctggactccatggcgtccacacagaatcgacacgagcttggtgtgccgcgtacgcatgtgtgcgtatgtatgcctcgtcttccacatgcaaacatacgcagaggaaggggaaaggcggcagcaaacgcgacggtccaagtcgtaccacagaagtggtcgcgcatgtgtgcccaagttgccatcacccggatgctattagatttccagaaactaacttgtgaggacccctggtgtctgctagctgctctccaactccaacctgtcaatcaattcccagacggacaagctgagctcacagctcaagctcaacaacgatggccggccgggtcaccatggaactgatcctctacagtacaggcatgggaaaatggaggaggagagcagggcagtgaggccacagaatcagaggctgattagtgttggtgagctccaatccaacagcatatgaccagcgagcagaacatagggatgtcctgtgggcttgcccagggacagacgcatgcaagccatgtgactgtccggagagagagccggtgatactggaacagaggatccgatcctgccccccttcttttgcctctccctctctcacacacacagtctcacctatatgtggctatgtcgtctccattaggctgttaactagccaacacatgttcccccgttgcttaagacagcagctacaaagcgagaacatcatgctctaaaaagaaacttccgcaatgcaccactagcacatgtctgcgcctcaattcgcaaccggcaagcaagcaagccggcaagcagacagtcgccatacggtttttaccaaacagctagcgcccacagctgactagctgaccaccgcaccacccacactcctcctcgcgagtcgcgaggcaagccgcaagctcctatatagagaggccccctccctccccctgcatggacagccaccgccttcttcaaccctccttccgtcttcctcctctagtcttacctcgttgcacctcaagaaacttggcgcgcaaccaggaaaccccctcttctctctctctctctctctctctctctgccttctgattccaagctccccaactgcccagcaccaacctgccgaactcccctcctttttgttggtttgtcgaattataaattgagcccggccggctgactacc

SEQ ID NO: 14 - DNA, 2001 bp, Zea mays
  N_region (0)...(0): ZmCkx3 promoter
ccggggtgtgacaggagcattgaagcatgcatgctctgctcagcatataattaaagaaagaagcatcaaaatgcactggagcagttgaccaaaacttgcagctacgtcaaaatatatacgagggctggcatcaaggtgtgctcagcccgagccccgtcaggtaacttggtcttttgttttctggccttgcggcttcattaaaggccgccggccgcgagcgaggcaaaacagtgaaggggaggggaggtgcccgccactaacctctcggtcggatatattagtattcaagcagttgacaaatctgtgcggatttgatttggtctgaggaaaatatatatatatatatatatagcccctcgtcgttcatgcaccctctcgcagcctgcaaccttacaatattgttcttgcatccggttttatttatatttttattttttaaaaaaaaaatccatagtcctgccgtcttgaaggatatgtttttctttacccatgcacggcggagtttaaatttgcgctgacccgactgctcgtgaacagagacaagtatgacagatatcgttgagttccaaattttaaaaaaaaaatcaataaaaaatttaaaacagaatgttgacgaggaaaaaaaatatgaaggtgcttgcacacctgtcactccatgccggacatcaacaaattaattgttcaagtggtgggagtcagctgcttccagtttaccttcctgcgccagcggttggtagacaggattgttgccacgtggacgaaatctcctgccgccagctggttgatcacggcaggcagtcacatgcttcttgccaagattaccgcgggttgtaatcatctgaaatatattaacctgagcacgtgatagagtaaaaaaattggtcgactaagggggtgtttggtttctagggactaatgtttagtccctacattttattccattttagttctaaaattaccaaatatagaaactaaaactttattttagtttctatattagcaatttatagactaaaaaagaataaaatgaagggactaaatattaatccctagaaaccaaacaccccctaactttaggtaagttgtggcatgcattctctggaacggcagttctagagagcacttgagatgtcaacaggtgaagaattgaagattggccaacacaggcgttcaaggagattcaaccacccatccacataccgcgcaaacacttggggggcattcttgctgctgccacatttggaagaagcgcagcaatgtggtgttcagaagaagcacagctattttagctcttgataactatctttttttttgcatagattaatttatttcttcgatatatactagcttgtaaaaaaatgttttncagatatatgtataaaaatgtgtacctagtacctacgcatgtcttagttcaacatacttgatagctgtagttttctgaaaacctgttcaaattaacctttttcctaccctgatggtgaatagagagaaaagctttacctttgtctgaataagaaaactaacagaaagcttacattttggccactctacctgcccgagtattttctaagcaagcaaaggcgcatgaaaattttctcggaatccatgaccttttacgcgcantgnwaaayawwgwmmattgmtcmgaccaatgatcattttgatactctccacaagtcaacatctcaaaaaaaccacaagatggggcccatcaacataagttcacgagtgtgccttcaggtacattgttctttttttttgttttgctaaagtcaatcagctgcaaaatattcagaacaatttcaataacccgaaaggctgttgtgcctccatttgtcaacgtttgcgaggccaaatggtacccccgctataaataccatggaagttcttggcctctaggacacacaagcgatctctcctcctatagtttctataaccccacaaagcgtccaggtcccgtagtcacctccgattgcattgcgttgccgcaagacaagc

SEQ ID NO: 15 - DNA, 2448 bp, Zea mays
  misc_feature (0)...(0): ZmCkx4 promoter
ctttatgttgtagccaaggaaagtatactgttaagatcagaatgaaccttataggagttgtatgggcataaagccagcaagtatagccaaaggtacacaaggctaatatagtcaagttgttgatgtgtgagacgttcaaggaagtgaactattggaggagtcgactaaaagtacgattaataaggtagacatgatggtaaaatctttgatctagaatttaagtggtatggatgcgagggtgagaatggcaagcacaacttcaaatatagggtgatgcttatgcttggctgagccatttcattcatgagcataggaacatgagacatggtgggatatggatacttgcacaaaaaaaggaattaagtttatgatattcacctcccagtcagtttgcatggtaaaaaaattcctatcaatttggttctcaactagggcctaaaattctcaaaatatctgttggggaccattatcgtcgacgatcctcagaatctgttattaccaaattaaaaggtgtgtttcaggtactgtgcaaagcagcagcgaagctatccttcgtcaaaagtggctcaatgaaccaggtggagaagctatggagcttcgtctgcgtagagcgtgccggaggaggaagctttggctctgaatgcatcgacttacgaagcatgggagaagaagactcagaaggcttgtccagcgtgggaataaaaaggagaaaatacaattttgcccttgtgggatttgtaaatcatgtgcaaggctcatggatatgtttgtaattttatatgatatgtttgtaaatcatggatatgttttgtaaatcaggtggactagaggagagggagggtggacatagtgacttgcatcttgatcatggtagagtggtcatggtagagggaaaggggtaggtcaattctggagtgcggccacggtggcttgagtgtcggccacggtaggggaaaggggtagcccaattctagggccggcatcggagaaggccgacatgtgcacgtcaggaggtagtgttagaggtttgaacggaaaaaattgaacatgttagtatgatgagttgtgtaattgctgggaattgtggataatttccacttaactacggccctgtttatttacccctagattataaaatccaacttaaaaaagttgagatgtaaacaaacaacacatattattaggtggattatgttatctagaaatctggatgataataatttataagtcggttaataggtgtttacataatcgataagctggattatataatcctggaacacggctttcgcgagagcgtattaaaacaggattccgtgaagcacactatctgaggagctccaccaaaagctgaatctagcccgcactcttttttggaggattcaaatttggtgtcactggagcattcggcattttgtttcatggcgtgaagctatttttactaattacagaagctgtttcaaatagacctttaaatgatggctgagtataaaaggaggcaatttttttatctcgccgatggagccaggtcgcgtcgcgccgcggccgtgctgcgctctcgacgcgatctagcggcgatgtgcacagtacagttttgccatgccattggttaagcctgcatacaacacaccagcgtactgccctgcacaagatctcctcggctcggcctctcctgatggaacgttcagcttgaacagcggagcgtgggggcatcccggggatgggcgccgcggccgagaaattttgcaacctggcaaatctgccctgtcgcatactaccatccacctccaggcgccaagaacgcctccgagtttcaggcttgcagctcagctctgtgttgaattggaacgggcggagtttctgggttccagacttccagtacaaggcgatcaattggtagggcgaattacttgcaggcccagatgcatggcccatctatctggttctctatcggttgcttttacttgcacaatagtggcagacaaactacaagtcagatccgatcctatccatccatccatctcgcagcgcgatgcaaatatgcaatcgtctgtggaactcgaaaaaaaacagaggtccggcctcgcacgaggttaagggaaaaaaaacgaagcgtttggaactttggttggcattcgcagcatgctgtgctgccaccgtatgtttttatttttgctttgtttgtcttctttgagaaacgtgagggagccgcgtgtccgctcgttataaaacccccccggcgacccaaactaccacgagctcaagcctcaagcctcaagcctcaagcaagcagagcgccgtgacatcacgaaacaaacatatagagctagctgctctgcctctgcttcaccaatcacctgcttggccgcgcggaggggagggtttccccctttgacacagctgagctcccctccatcagcagccagctcctcgtcgcaaagcaagaag

SEQ ID NO: 16 - DNA, 2346 bp, Zea mays
  misc_feature (0)...(0): ZmCkx5 promoter
tacagatttgcgttcatcaatggcagcgcgggatctcatgaggtcactgggttcttgcaagtggggagagaaagggagatctacgaaagacttgttagtgggccaccttttccctctttccccacaaggacgagatcgtggattagagtaggaaagtgattccgcattggtctcaaatcttggcgaaagattgcattgtgtactctccaccactcgaccggcaacgaggcattttgttattgcacgatgcatcctttgcacatgagctaggcttgtgcctttgagtattcagttagcattgcaaccccatttcaattcacatgcttgtctttccaaggaactttctaagccacctaacagacattagggtttatatcagaatcgagctcatggcgtactttatgctgcacgaacaatgggttgggggcgtcgtttcttgcatgagagcatgcgcatcctggtaaggatttcgccaaaagaactttagtcctctaccgactttgtgtttgcgtgatctcgtgatttgaagcctgtggtggtgtgctgaggcagcatattggaaggtatctctgtgttgatatggcatccgtccgtggacaaatcgataccacatactgttcttggattctattcttgggattgctaaatgatctagatagattatattctcttgttgcagcccctattgcttcaatacgaagaaaacccaacgtttagaacttaataaaaccatttgtgagcttagctgcttaggcaattcatttttatgcatgacaaatatataataatattagctatactattattgatgcaacctgtgggagcgtataaaatggtacttccccaattctaaattataagacgttttgactatatattctacatacatatgtttaattttatatttagataatcgctatgccttaatatatagtaaaaagtagtatatctagaaaagataaaacatcttataatttaaaaatgggtagagtattatattagatatgaacagtgcttagatgccaccaaaattttgccatgccatcctaaggccagcaaaagtttgtgtcttcttttgttttccaaaccactagatgccaatatactatttatcatcgatcgagatgtaggtcttagttaattgtgtcgggtgcccttgagaaagaaaagaaaaaggtgggattttgttttcgcttagacgatgattggatctcttggtctctgaattccatcccgaataaacaaatgaagtaggtcctcagtcacccttgccctgttagctgcaagagagctcatggtttccagccacacaatcagtccatggctccttcttcttggcctaagtggtggccaatcattgtgggtgatcgagtcttgggccctctgaacagtattacacaacagtaatcctgcaaaagatttggtatatctagattctagagtgagcgccgtgttgtgcccagctaggaatgggttgtcaagtgcaacaggaggaggacccaggatggtcaggtgtaataggctctcattaaaagactgttcagatggattagagcaacgacggggaagccgggaaaaaatggttggttctgctttcctctcgctccccggccgggttcatatatgaatctgagaacgatattttttgcttcatttttcatttgctatatatttaaactgtttttttgtgtgtgtgtgtgtgttcattgagctcaatacttgaggcttgatagggagaggagtgaggcagctgatcacatggacctccatctgaggacagttcctcttccgaaacagaaaggagagtgcagggaccagcgtggcctgtacagtattgtgtttgcccttttcctttggcagggacagagagcttcaggcttgtcctctttatgtatgctgctcgcctgcttcagagtcagagcttccccttctcacttctcagagagagagagagagaagagagagagaggagagccctccacagctcccctgtcctgccctcaggcattctttgtcacagggggcgagggctgaagatcatcacatggtggccttttttgggtctgtggcctttggtcttttagtgcttcttccttttacctcctcatgacatgaaccccctttttaaacctccctcaaaatcaaatcaccctccttctcctttaagagccctcaaccccttcccctcattttccttcatccctcagcctttgcacaaagggcaagaataacgcagtatgatcatctgatcatactcccgccgccatcacaatcccacacgaacgtgagacaaaggtaacagacgcaagaagctagcagctgcaggagattgctcagcccatctcc

SEQ ID NO: 17 - DNA, 1470 bp, Zea mays
  misc_feature (0)...(0): ZmCkx1-2 promoter
gagctcgcccttgcatgcttgagtcatatcttggaaaaaaaaactgtaacttaaagtatgatctatatatggattatttggatgggatgtcattttcgtatcaccaaccaaaattacagtttggtcgtgcgtagaaattctacctactagctgaaacaacggctgctatgtataactactggtactggaaagaatattagtcattgactcaaaattagaatgcatgtgtaagtcatgcgtgctaatttgttctatcagcattcggcgaattccgaagtccgtacgtgttgttcgtggaggagaggaaaacatcagaaatgacaaaactagacggcgtgtgcttctacactgaattcatcaacatttgttttacttttactagagaatggcatcagatggaaaaccgctgaaaaaacaagaaaacaattggaccccaaatatgtacagacgctagctatagccagccacactgaagttgacatgcggcaactagctaaccaccttctctgaaacactaacatttgtaccttggtcgtgtaagtgtagttagtaacgtatgttgacgcgacttaccgaacaaaaatataattgtcccaatcaagctagggacgattgtttgtttccaaaatgttgccatttgcttaatcaatcctatattgattcatggctgttaaggtgagataaagcgacaagaaatctctctctatatatatatataagatcccgaaggctagcgacatttttgatagcaaaatatgagaagttggcaggttctggtagcaaatcaaataatatggccagaataatcgtggctagcttgattaaaccttcagcttggtgtattttggaagtcgaccaaccagctgggccggggctcgtcgtagtaccaaaattacagcctgcttccttcgtcgtcctgtacgtaatgcagtacagctgtctgtctagtagagacgattttgagcaggcacacacattaagtgataacataaaagacggcttcattttatttcataaccaaacgatatggtcaacacacacctatagctaccaaatttgtacaactatttagtgcgaaaactatttcattctcaagaattgatcgcttatatttattattacaggtttttaaatgtataaatacgctatattgcatggcaaaagggggtaataattaggcaggactatatatataatagttttttttcctttaaattcttgggaggatggtaaagttggtaactaggcaccttgtgcgcatatttttctgtggtcaaacagaataaaactagacgggatgcagaatttttttttccttggaaagcagctcatctctgtgttcgagtacgtaattgaagaagtatgtgatcgcactacacctacacgtatgtgccgccgtatccgtcctatatatatacggggtgcaatcacctagttaccaaacactcacacataagggcggatccatgg

SEQ ID NO: 18 - DNA, 3317 bp, Zea mays
  misc_feature (0)...(0): ZmCkx1-2 promoter extended
tcttgttcccaagtgttttgtaagcaaggcaagagacaccaagtgtgtggtggtccttgcggggtctaagtgacccatttgattaaggagaaggctcactcggtctaagtgaccgtttgagagagtgaaagggttgaaagagacccggtctttgtgaccacctcaatggggactaggttctttagaaccgaacctcggtaaaagaaatcatcgtgttcatccgctttatttcttggttgatttgtttttcccctctctcccgaactcggatttaattctaacgctaaccccggcttgtagtttatgttttaagttgtaaatttcagattacgcctattcaccccctaggcaactttcagttccaccactcctccccaccttgtacttacgaaagattgtgttagtagatctcgagatttacaaacttaccataagcaactaaaaataatataaacctaaaaatatgaaaacccggaaagagcctaaaacttgaacccggaaaaaaattcctgcaataatcataatcaactaagttatgctcacacattaggtctgcgttgttgaattaactttcaagaccagaaaacaggcacgcgtgcatcgtacaagtggaacttccaaacctttaagtaaatagagatagacacgcccacatgtggaacttcttggtacgtaatgcgtgacaagttagaagtgaacaaggcaaccacgtcgctacttgtgatgaatactgggctgtgtgtgggttttgtcgtcgcaggagtacatgtgttctctgaattctaaatcgatcatgatgtccgtcttttttttattttagcccttttttagttttttttcataaatatgccctttctagttttaactcaaaaaatggaccctctggtcgacaccattactattggcggcaacctaacacgtctaggcgcctagacggctagaccttgttcgtggcattgacgtggtgcacccgtggctagtgaggtggcagaggtaggcgctaagaactatagtgccgaaggtaggcggcatatatcttggcacaggccccctttaatacgggcccacgtgtaattttattttccttttcctcactctccaccctcgtcgttcgcctggctcaacatagccgcccctctccctctccgcgtctcgccatcgtcgcccctctcctcgcagtgacttgccatggtcgcggctcaccgtggccacgccatgccagcgtcagcccgctccctcatttatcgatgcgggctcggtggtcggcgacgagcgcaaggctgaggagcgtgggcgggcgacggtggtgagcgacgtaggacgccgacgacggtgacgggcgacttagggcaccgacgacgtatggcgtgggcgggcgccagcggcctcaccgacggaggatggcgcgggcaacgctggagagtggccgagcagcgaccgggtcgaatggaagagagagaaagaggaaaagaacgtgaaccgattggtatatagagggtcggcgctaagatctatggcgtcgatccataccatgcctcagggtccttcctggccacgtcatcgatacatatgcgtcaattgcattggcaccaatacgtgttaggttggcaccaatagtgagagcgtcaacctaagggtccatattctgaattcaaacaaaaaataacctatttgtgaaaatgaaacactaaaaaggctaaaatagaaaaaaaatcggtcgcgatgcagacgcatcgtcggtttcaccgccgtcacgcgcgcgtctgtatgcgctgccaggagtcactgcaagcggcaagcagccaaaaaaataaaaattggctgcatccgatctcgagactccgacgagaggaggctgcgcatgcttgagtcatatcttggaaaaaaaaactgtaacttaaagtatgatctatatatggattatttggatgggatgtcattttcgtatcaccaaccaaaattacagtttggtcgtgcgtagaaattctacctactagctgaaacaacggctgctatgtataactactggtactggaaagaatattagtcattgactcaaaattagaatgcatgtgtaagtcatgcgtgctaatttgttctatcagcattcggcgaattccgaagtccgtacgtgttgttcgtggaggagaggaaaacatcagaaatgacaaaactagacggcgtgtgcttctacactgaattcatcaacatttgttttacttttactagagaatggcatcagatggaaaaccgctgaaaaaacaagaaaacaattggaccccaaatatgtacagacgctagctatagccagccacactgaagttgacatgcggcaactagctaaccaccttctctgaaacactaacatttgtaccttggtcgtgtaagtgtagttagtaacgtatgttgacgcgacttaccgaacaaaaatataattgtcccaatcaagctagggacgattgtttgtttccaaaatgttgccatttgcttaatcaatcctatattgattcatggctgttaaggtgagataaagcgacaagaaatctctctctatatatatatataagatcccgaaggctagcgacatttttgatagcaaaatatgagaagttggcaggttctggtagcaaatcaaataatatggccagaataatcgtggctagcttgattaaaccttcagcttggtgtattttggaagtcgaccaaccagctgggccggggctcgtcgtagtaccaaaattacagcctgcttccttcgtcgtcctgtacgtaatgcagtacagctgtctgtctagtagagacgattttgagcaggcacacacattaagtgataacataaaagacggcttcattttatttcataaccaaacgatatggtcaacacacacctatagctaccaaatttgtacaactatttagtgcgaaaactatttcattctcaagaattgatcgcttatatttattattacaggtttttaaatgtataaatacgctatattgcatggcaaaagggggtaataattaggcaggactatatatataatagttttttttcctttaaattcttgggaggatggtaaagttggtaactaggcaccttgtgcgcatatttttctgtggtcaaacagaataaaactagacgggatgcagaatttttttttccttggaaagcagctcatctctgtgttcgagtacgtaattgaagaagtatgtgatcgcactacacctacacgtatgtgccgccgtatccgtcctatatatatacggggtgcaatcacctagttaccaaacactcacacataagggcggatccatgg

SEQ ID NO: 19 - DNA, 25 bp, Artificial Sequence
  primer
ctgactaccatgaagccgccatcat

SEQ ID NO: 20 - DNA, 29 bp, Artificial Sequence
  primer
ttgctctggttatgatcccgaactgacca

SEQ ID NO: 21 - DNA, 24 bp, Artificial Sequence
  primer
atcctcaacaactggagggcgtcc

SEQ ID NO: 22 - DNA, 27 bp, Artificial Sequence
  primer
tcccaccttgctccaaagtgggttttc

SEQ ID NO: 23 - DNA, 27 bp, Artificial Sequence
  Primer
cgcaggtcagcaatgtcaatcaactgg

SEQ ID NO: 24 - DNA, 27 bp, Artificial Sequence
  Primer
agaggtgtgcaccctgtccaaaaactc

SEQ ID NO: 25 - DNA, 32 bp, Artificial Sequence
  Primer
agagaagccaacgccawcgcctcyatttcgtc

SEQ ID NO: 26 - DNA, 24 bp, Artificial Sequence
  Primer
ggagggtttccccctttgacacag

SEQ ID NO: 27 - DNA, 27 bp, Artificial Sequence
  Primer
gggacgacaaagagcgcgatatgatga

SEQ ID NO: 28 - DNA, 28 bp, Artificial Sequence
  Primer
acgcacgcttcggcggcattcattattt

SEQ ID NO: 29 - DNA, 28 bp, Artificial Sequence
  Primer
ggcaagcatgcatggagcaaaaactcgt

SEQ ID NO: 30 - DNA, 29 bp, Artificial Sequence
  Primer
gcactactaactgactgacgttgccattc

SEQ ID NO: 31 - DNA, 28 bp, Artificial Sequence
  Primer
gcaactcacttgctcttgagcatagggt

SEQ ID NO: 32 - DNA, 32 bp, Artificial Sequence; identical to SEQ ID NO: 25
  Primer

SEQ ID NO: 33 - protein, 752 aa, Zea mays
MAVVYYLLLAGLIACSHALAAGTPALGDDRGRPWPASLAALALDGKLRTDSNATAAASTDFGNITSALPAAVLYPSSTGDLVALLSAANSTPGWPYTIAFRGRGHSLMGQAFAPGGVVVNMASLGDAAAPPRINVSADGRYVDAGGEQVWIDVLRASLARGVAPRSWNDYLYLTVGGTLSNAGISGQAFRHGPQISNVLEMDVITGHGEMVTCSKQLNMAVVYYLLLAGLIACSHALAAGTPALGDDRGRPWPASLAALALDGKLRTDSNATAAASTDFGNITSALPAAVLYPSSTGDLVALLSAANSTPGWPYTIAFRGRGHSLMGQAFAPGGVVVNMASLGDAAAPPRINVSADGRYVDAGGEQVWIDVLRASLARGVAPRSWNDYLYLTVGGTLSNAGISGQAFRHGPQISNVLEMDVITGHGEMVTCSKQLNADLFDAVLGGLGQFGVITRARIAVEPAPARARWVRFVYTDFAAFSADQERLTAPRPGGGGASFGPMSYVEGSVFVNQSLATDLANTGFFTDADVARIVALAGERNATTVYSIEATLNYDNATAAAAAVDQELASVLGTLSYVEGFAFQRDVAYAAFLDRVHGEEVALNKLGLWRVPHPWLNMFVPRSRIADFDRGVFKGILQGTDIVGPLIVYPLNKSMWDDGMSAATPSEDVFYAVSLLFSSVAPNDLARLQEQNRRILRFCDLAGIQYKTYLARHTDRSDWVRHFGAAKWNRFVEMKNKYDPKRLLSPGQDIFN

SEQ ID NO: 34 - protein, 579 aa, Artificial Sequence
  Consensus Sequence
VXXFLLLVLLLLSSLALAAGXPXDLXXLGXXXXGXLXGXXXXXRLXTDAXSTAAAATDFGNIXSALPAAVLHPASXGDIAALVRAAXXSXSASPLTVAARGXGHSIXGQAQAPGGVVVDMXSLGGAXXXXXXXXIXXXXVAGGGRXXFVDVGGGXLWIDVLRXTLKHGXLAPRSWTDYLYLTVGGTLSNAGISGQAFRHGPQISNVXELDVVTGRGEMVTCSPSXNADLFFAVLGGLGQFGIITRARIALEPAPKRVRWVRVLYSDFAAFTADQEXLISXXXXXXXAXXXSFDYVEGFVVLNXSLIXNXXXSSFFSPADVXRLXSLASXSXXXVLYCLEVTLNYXXGTAXSXXXXXXVDQEVAALLGXLSFVXGXLFTXDVTYVDFLDRVHXEELXLRAXGLWEVPXHPWLNLFVPRSRIADFDRGVFKGILXDXXXXXXGPILIYPMNKSKWDXRTSVVTPDXXXXXXEEVFYLVAFLRSALSGXXXXXXXXXXXSLXXLLRQNRRILDFCDAAGIGXKQYLPHHTTXADWXRHFGAARWERFADRKARYDPLAILAPGQRIFPRXXXXAAIAXAXXX

SEQ ID NO: 35 - protein, 575 aa, Arabidopsis thaliana
MGLTSSLRFHRQNNKTFLGIFMILVLSCIPGRTNLCSNHSVSTPKELPSSNPSDIRSSLVSLDLEGYISFDDVHNVAKDFGNRYQLPPLAILHPRSVFDISSMMKHIVHLGSTSNLTVAARGHGHSLQGQALAHQGVVIKMESLRSPDIRIYKGKQPYVDVSGGEIWINILRETLKYGLSPKSWTDYLHLTVGGTLSNAGISGQAFKHGPQINNVYQLEIVTGKGEVVTCSEKRNSELFFSVLGGLGQFGIITRARISLEPAPHMVKWIRVLYSDFSAFSRDQEYLISKEKTFDYVEGFVIINRTDLLNNWRSSFSPNDSTQASRFKSDGKTLYCLEVVKYFNPEEASSMDQETGKLLSELNYIPSTLFSSEVPYIEFLDRVHIAERKLRAKGLWEVPHPWLNLLIPKSSIYQFATEVFNNILTSNNNGPILIYPVNQSKWKKHTSLITPNEDIFYLVAFLPSAVPNSSGKNDLEYLLKQNQRVMNFCAAANLNVKQYLPHYETQKEWKSHFGKRWETFAQRKQAYDPLAILAPGQRIFQKTTGKLSPIQLAKSKATGSPQRYHYASILPKPRTV

SEQ ID NO: 36 - protein, 501 aa, Arabidopsis thaliana
MANLRLMITLITVLMITKSSNGIKIDLPKSLNLTLSTDPSIISAASHDFGNITTVTPGGVICPSSTADISRLLQYAANGKSTFQVAARGQGHSLNGQASVSGGVIVNMTCITDVVVSKDKKYADVAAGTLWVDVLKKTAEKGVSPVSWTDYLHITVRGTLSNGGIGGQVFRNGPLVSNVLELDVITGKGEMLTCSRQLNPELFYGVLGGLGQFGIITRARIVLDHAPKRAKWFRMLYSDFTTFTKDQERLISMANDIGVDYLEGQIFLSNGVVDTSFFPPSDQSKVADLVKQHGIIYVLEVAKYYDDPNLPIISKVIDTLTKTLSYLPGFISMHDVAYFDFLNRVHVEENKLRSLGLWELPHPWLNLYVPKSRILDFHNGVVKDILLKQKSASGLALLYPTNRNKWDNRMSAMIPEIDEDVIYIIGLLQSATPKDLPEVESVNEKIIRFCKDSGIKIKQYLMHYTSKEDWIEHFGSKWDDFSKRKDLFDPKKLLSPGQDIF

SEQ ID NO: 37 - protein, 523 aa, Arabidopsis thaliana
MASYNLRSQVRLIAITIVIIITLSTPITTNTSPQPWNILSHNEFAGKLTSSSSSVESAATDFGHVTKIFPSAVLIPSSVEDITDLIKLSFDSQLSFPLAARGHGHSHRGQASAKDGVVVNMRSMVNRDRGIKVSRTCLYVDVDAAWLWIEVLNKTLELGLTPVSWTDYLYLTVGGTLSNGGISGQTFRYGPQITNVLEMDVITGKGEIATCSKDMNSDLFFAVLGGLGQFGIITRARIKLEVAPKRAKWLRFLYIDFSEFTRDQERVISKTDGVDFLEGSIMVDHGPPDNWRSTYYPPSDHLRIASMVKRHRVIYCLEVVKYYDETSQYTVNEEMEELSDSLNHVRGFMYEKDVTYMDFLNRVRTGELNLKSKGQWDVPHPWLNLFVPKTQISKFDDGVFKGIILRNNITSGPVLVYPMNRNKWNDRMSAAIPEEDVFYAVGFLRSAGFDNWEAFDQENMEILKFCEDANMGVIQYLPYHSSQEGWVRHFGPRWNIFVERKYKYDPKMILSPGQNIFQKINSS

SEQ ID NO: 38 - protein, 524 aa, Arabidopsis thaliana
MTNTLCLSLITLITFFISLTPTLIKSDEGIDVFLPISLNLTVLTDPFSISAASHDFGNITDENPGAVLCPSSTTEVARLLRFANGGFSYNKGSTSPASTFKVAARGQGHSLRGQASAPGGVVVNMTCLAMAAKPAAVVISADGTYADVAAGTMWVDVLKAAVDRGVSPVTWTDYLYLSVGGTLSNAGIGGQTFRHGPQISNVHELDVITGKGEMMTCSPKLNPELFYGVLGGLGQFGIITRARIALDHAPTRVKWSRILYSDFSAFKRDQERLISMTNDLGVDFLEGQLMMSNGFVDTSFFPLSDQTRVASLVNDHRIIYVLEVAKYYDRTTLPIIDQVIDTLSRTLGFAPGFMFVQDVPYFDFLNRVRNEEDKLRSLGLWEVPHPWLNIFVPGSRIQDFHDGVINGLLLNQTSTSGVTLFYPTNRNKWNNRMSTMTPDEDVFYVIGLLQSAGGSQNWQELENLNDKVIQFCENSGIKIKEYLMHYTRKEDWVKHFGPKWDDFLRKKIMFDPKRLLSPGQDIFN

SEQ ID NO: 39 - protein, 524 aa, Arabidopsis thaliana
MIAYIEPYFLENDAEAASAATAAGKSTDGVSESLNIQGEILCGGAAADIAGRDFGGMNCVKPLAVVRPVGPEDIAGAVKAALRSDKLTVAARGNGHSINGQAMAEGGLVVDMSTTAENHFEVGYLSGGDATAFVDVSGGALWEDVLKRCVSEYGLAPRSWTDYLGLTVGGTLSNAGVSGQAFRYGPQTSNVTELDVVTGNGDVVTCSEIENSELFFSVLGGLGQFGIITRARVLLQPAPDMVRWIRVVYTEFDEFTQDAEWLVSQKNESSFDYVEGFVFVNGADPVNGWPTVPLHPDHEFDPTRLPQSCGSVLYCLELGLHYRDSDSNSTIDKRVERLIGRLRFNEGLRFEVDLPYVDFLLRVKRSEEIAKENGTWETPHPWLNLFVSKRDIGDFNRTVFKELVKNGVNGPMLVYPLLRSRWDDRTSVVIPEEGEIFYIVALLRFVPPCAKVSSVEKMVAQNQEIVHWCVKNGIDYKLYLPHYKSQEEWIRHFGNRWSRFVDRKAMFDPMAILSPGQKIFNRSL

SEQ ID NO: 40 - protein, 540 aa, Arabidopsis thaliana
MNREMTSSFLLLTFAICKLIIAVGLNVGPSELLRIGAIDVDGHFTVHPSDLASVSSDFGMLKSPEEPLAVLHPSSAEDVARLVRTAYGSATAFPVSARGHGHSINGQAAAGRNGVVVEMNHGVTGTPKPLVRPDEMYVDVWGGELWVDVLKKTLEHGLAPKSWTDYLYLTVGGTLSNAGISGQALHHGPQISNVLELDVVTGKGEVMRCSEEENTRLFHGVLGGLGQFGIITRARISLEPAPQRVRWIRVLYSSFKVFTEDQEYLISMHGQLKFDYVEGFVIVDEGLVNNWRSSFFSPRNPVKISSVSSNGSVLYCLEITKNYHDSDSEIVDQEVEILMKKLNFIPTSVFTTDLQYVDFLDRVHKAELKLRSKNLWEVPHPWLNLFVPKSRISDFDKGVFKGILGNKTSGPILIYPMNKDKWDERSSAVTPDEEVFYLVALLRSALTDGEETQKLEYLKDQNRRILEFCEQAKINVKQYLPHHATQEEWVAHFGDKWDRFRSLKAEFDPRHILATGQRIFQNPSLSLFPPSSSSSSAASW

SEQ ID NO: 41 - protein, 504 aa, Arabidopsis thaliana
MLIVRSFTILLLSCIAFKLACCFSSSISSLKALPLVGHLEFEHVHHASKDFGNRYQLIPLAVLHPKSVSDIASTIRHIWMMGTHSQLTVAARGRGHSLQGQAQTRHGIVIHMESLHPQKLQVYSVDSPAPYVDVSGGELWINILHETLKYGLAPKSWTDYLHLTVGGTLSNAGISGQAFRHGPQISNVHQLEIVTGKGEILNCTKRQNSDLFNGVLGGLGQFGIITRARIALEPAPTMDQEQLISAQGHKFDYIEGFVIINRTGLLNSWRLSFTAEEPLEASQFKFDGRTLYCLELAKYLKQDNKDVINQEVKETLSELSYVTSTLFTTEVAYEAFLDRVHVSEVKLRSKGQWEVPHPWLNLLVPRSKINEFARGVFGNILTDTSNGPVIVYPVNKSKWDNQTSAVTPEEEVFYLVAILTSASPGSAGKDGVEEILRRNRRILEFSEEAGIGLKQYLPHYTTREEWRSHFGDKWGEFVRRKSRYDPLAILAPGHRIFQKAVSYS

SEQ ID NO: 42 - protein, 536 aa, Dendrobium sonia
MNLHAMPPFLNPTSLLLTTTLMSILIQSPNSLPTNLLTHPTSTHLRFDSLSLSAASSDFGDIIHSLPSAVFLPSSPSDIATLLRLSHFSPHSFTVSARGLGHSTRGQAQAFGGIVINMPSLDGGITVSIDGMFVDAGAEQMWIDVLRETLRHGLTPKSWTDYLYLTLGGTLSNGGISGQAFLHGPQISNVHELDIVTGKGEMVTCSESNNPDLFFSVLGGLGQFGIITRARIALEKAPQSVRWMRLMYTDFELFTKDQELLISIKAEGEGWKLNYVEGSLLMEHSLKSNWRSPFFSEKDLKKIKKLASGNEGVIYCLEASFYYDYGHEMNFSRADKAQMDQDIEELLRKLSFVSGFAFRNDVSYMGFLNRVHDGELKLRAMGLWDVPHPWLNLFVSKSNIMDFHIGVFKGIMKNSKSMGPILVYPTKRSKWDKRMSTSIPDEEVFYSIGILLSSEMNDLEHLESHNAEILKFCDQQGMNYKQYLPHYTSIEDWKKHFGKKWERFVEMKSRYDPKAILSPGQKIFTHLVDELCLSDH

SEQ ID NO: 43 - protein, 526 aa, Hordeum vulgare
MRQLLLQYLKLFLLLGLGAVTAEHVLKHDVLASLGTLPLDGHFSFHDLSAAAMDFGNLSSFPPVAVLHPGSVADIATTVRHVFLMGEHSALTVAARGHGHSLYGQSQAAGGIVIRMESLRSVKMQVHPGASPYVDASGGELWINVLNKTLKYGLAPKSWTDYLHLTVGGTLSNAGVSGQTFRHGPQISNVNELEIVTGRGDIVTCSPEQNSDLFRAALGGLGQFGIITRARIALEPAPQMVRWIRVLYLDFMSFTEDQEMLISAEKTFDYIEGFVIINRTGILNNWRSSFNPQDPERASRFETDRKVLFCLEMTKNFNPEEADIMEQEVHALLSQLRYTPASLFHTDVTYIEFLDRVHSSEMKLRAKGLWEVPHPWLNLIIPRSTIHTFAEQVFGKILEDNNNGPILLYPVKKSRWDNRTSVVIPDEEVFYLVGFLSSAIGPHSIEHTLNLNNQIIEFSNKASIGVKQYLPNYTTEPEWKAHYGARWDAFQQRKNTYDPLAILAPGQKIFQKKPASLPLSSLQYLL

SEQ ID NO: 44 - protein, 520 aa, Hordeum vulgare
MKQLLLQYLKLFLLLGLSRVTTEHVPKYDVLASLGTLPLDGHFSFHDLPAAARDFGNLSSFPPVAVLHPGSVADIARTVRHVFLMGEHSTLTVAARGHGHSLYGQSQAAGGIVIRMESLQSVKMQVHPGASPYVDASGGELWINVLNKTLKYGLAPKSWTDYLHLTVGGTLSNAGVSGQTFRHGPQISNVNELEIVTGRGDIITCSPEQNSDLFHAALGGLGQFGIITRARIALEPAPQMVRWIRVLYLDFMSLTEDQEMLISAEKTFDYIEGFVSINRTGILNNWRSSFNPQDPERASQFETDRKVLFCLEMTKNFNPEEAGIMEQIHALLSQLRYTPPSLFHTDVTYMEFLDRVHSSEIKLRAKGLWEVPHPWLNLIIPRSTVHTFAKQVFGKILEDNNNGPILLYPVNKSRWDNRTSVVLPDEEVSYLVGFLPSAMGPHSIKRTLNLNNQIIEFSNKASIGVKQYLPHYSTEPEWKAHYGARWDAFQQRKNTYDPLAILAPGQRIFQKTPASLPLSS

SEQ ID NO: 45 - protein, 532 aa, Oryza sativa
MAAIYLLIAALIASSHALAAHGAGGGVPLAAAAPLPFPGDLAASGKLRTDPNATVPASMDFGNITAALPAAVLFPGSPGDVAELLRAAYAAPGRPFTVSFRGRGHSTMGQALAAGGVVVHMQSMGGGGAPRINVSADGAYVDAGGEQLWVDVLRAALARGVAPRSWTDYLHLTVGGTLSNAGVSGQTYRHGPQISNVLELDVITGHGETVTCSKAVNSDLFDAVLGGLGQFGVITRARVAVEPAPARARWVRLVYADFAAFSADQERLVAARPDGSHGPWSYVEGAVYLAGRGLAVALKSSGGFFSDADAARVVALAAARNATAVYSIEATLNYAANATPSSVDAAVAAALGDLHFEEGFSFSRDVTYEEFLDRVYGEEEALEKAGLWRVPHPWLNLFVPGSRIADFDRGVFKGILQTATDIAGPLIIYPVNKSKWDAAMSAVTPEGEEEVFYVVSLLFSAVANDVAALEAQNRRILRFCDLAGIGYKAYLAHYDSRGDWVRHFGAKWDRFVQRKDKYDPKKLLSPGQDIFN

SEQ ID NO: 46 - protein, 558 aa, Oryza sativa
MAVLLMLNCFVKATAPPPWPPSASSASFLDDLGDLGIAPLIRADEAGTARASADFGNLSVAGVGAPRLAAAAAVLYPSRPADIAALLRASCARPAPFAVSARGCGHSVHGQASAPDGVVVDMASLGRLQGGGARRLAVSVEGRYVDAGGEQLWVDVLRASMAHGLTPVSWTDYLHLTVGGTLSNAGISGQAFRHGPQISNVLELDVITGVGEMVTCSKEKAPDLFDAVLGGLGQFGVITRARIPLAPAPARARWVRFVYTTAAAMTADQERLIAVDRAGGAGAVGGLMDYVEGSVHLNQGLVETWRTQPQPPSPSSSSSSSFFSDADEARVAALAKEAGGVLYFLEGAIYFGGAAGPSAADVDKRMDVLRRELRHERGFVFAQDVAYAGFLDRVHDGELKLRAAGLWDVPHPWLNLFLPRSGVLAFADGVFHGILSRTPAMGPVLIYPMNRNKWDSNMSAVITDDDGDEVFYTVGILRSAAAAGDVGRLEEQNDEILGFCEVAGIAYKQYLPYYGSQAEWQKRHFGANLWPRFVQRKSKYDPKAILSRGQGIFTSPLA

SEQ ID NO: 47 - protein, 527 aa, Oryza sativa
MEVAMVCTRVNLLILILSLCSPYKFIQSPMDFGPLNLLPTTTTASSDFGRILFHSPSAVLKPQAPRDISLLLSFLSASPLGKVTVAARGAGHSIHGQAQALDGIVVEMSSLPSEIEFYRRGEGDVSYADVGGGIMWIELLEQSLKLGLAPRSWTDYLYLTIGGTLSNAGISGQTFKHGPQISNVLQLEVVTGRGEIVTCSPTKDAELFNAVLGGLGQFGIITRARILLQEAPQKVKWVRAFYDDFATFTKDQELLVSMPVLVDYVEGFIVLNEQSLHSSSIAFPTNVDFNPDFGTKNNPKIYYCIEFAVHDYQNKNINVEQVVEVISRQMSHIASHLYSVEVSYFDFLNRVRMEEMSLRNSGLWEVHHPWLNMFVPSAGISDFRDLLMDSISPDNFEGLILIYPLLRHKWDTNTSVVLPDSGSTDQVMYAVGILRSANPDDGCSHHCLQELLLRHRRLAGAAASGLGAKQYLAHHPTPAGWRRHFGRRWERFADRKARFDPRCILGPGQGIFPRDSSSSNGAFASYS

SEQ ID NO: 48 - protein, 525 aa, Oryza sativa
MKPSIVHCLKLLMLLALGGVTMHVPDEDDVVASLGALRLDGHFSFDDAHAAARDFGNRCSLLPAAVLHPGSVSDVAATVRRVFQLGRSSPLTVAARGHGHSLLGQSQAAGGIVVKMESLAAAAARAVRVHGGASPHVDAPGGELWINVLHETLKHGLAPRSWTDYLHLTVGGTLSNAGVSGQAFRHGPQVSNVNQLEIVTGRGEVVTCSHEVNSDLFYAALGGLGQFGIITRARIALEPAPKMVRWIRVLYSDFETFTEDQEKLIASEKTFDYIEGFVIINRTGILNNWRTSFKPQDPVQASQFQSDGRVLYCLELTMNFNHDEADIMEQEVGALLSRLRYISSTLFYTDVTYLEFLDRVHTSELKLRAQGLWEVPHPWLNLLIPRSTVHKFAKEVFGKILKDSNNGPILLYPVNRTKWDNRTSVVIPDEEIFYLVGFLSSAPSSSGHGSVEHAMNLNNKIVDFCEKNGVGMKQYLAPYTTQKQWKAHFGARWETFERRKHTYDPLAILAPGQRIFPKASLPMSL

SEQ ID NO: 49 - protein, 532 aa, Oryza sativa
MAWCLVFMVFLIYCLISTVGLPVAPADEAAMQLGGVGGGRLSVEPSDVMEASLDFGRLTSAEPLAVFHPRGAGDVAALVKAAYGSASGIRVSARGHGHSISGQAQAAGGVVVDMSHGWRAEAAERTLPVYSPALGGHYIDVWGGELWIDVLNWTLAHGGLAPRSWTDYLYLSVGGTLSNAGISGQAFHHGPQISNVYELDVVTKGEVVTCSESNNPDLFFGALGGLGQLGIITRARIALEPAPHRVRWIRALYSNFTEFTADQERLISLQHGGRRFDYVEGFVVAAEGLINNWRSSFFSPQNPVKLSSLKHHSGVLYCLEVTKNYDDSTAVTVDQDVEALLGELNFIPGTVFTTDLPYVDFLDRVHKAELKLRGKGMWEVPHPWLNLFVPASRIADFDRGVFRGVLGSRTAGGPILIYPMNRHWDPRSSVVTPEEDVFYLVAFLRSAVPGSTDPAQSLEALERQNREILEFCDEAGIGAKQYLPNHKAQREWEAHFGARWARFARLKAEFDPRAMLATGQGIFDSPPLLAES

SEQ ID NO: 50 - protein, 650 aa, Artificial Sequence
  Consensus sequence
XXXXXXXXXXXXXXXXLLXXXLLXLLXXLXXXXAXXXXXXXXXSXLXXLXXXXXXXXXXXXXXXXXXLXXXXXXVAAASXDFGNIXXXXPXXXXXXXXAAVLHPXSXXDIAXLLRXAXXSXXXXXXXXXXXSXLTVAARGXGHSLXGQAXAXGXGVVVXMXSLXXXXXXXXXVXXVXXXXXGXXXXXYVDVXGGXLWIDVLRXTLKHGXLAPRSWTDYLHLTVGGTLSNAGISGQAFRHGPQISNVXELDVVTGKGEIVTCSXXXNSDLFFAVLGGLGQFGIITRARIALEPAPXRVRWIRVLYSDFXXFTXDQEXLISXXXXXXXXXXXXXFDYVEGFVIINXXXXXXXXXXXXXXXILNXWRSSFFXPXDXXRISXLXXXXXXVLYCLEVAKYYXXXXXXXXXXXXXXXVDQEVEXLLXXLXFIXGXLFXXDVTYVDFLDRVHXXELKLRAXGLWXEVPHPWLNLFVPRSXIXDFXXGVFKGILXXXXXXGGPILIYPVNRSKWDXRTSVVIPXXXXXXDEEVFYLVGLLXSAXXXXXXXXXXXXXXVEXLLXXNXRILXFCEXAGIGVKQYLPHYTTXXEWXRXHFGXARWDRFXXRKARYDPKAILAPGQRIFQKXXXXXXXXXXXXXXXXXXXXXXXXXXXXXXX

SEQ ID NO: 51 - DNA, 7366 bp, Zea mays
  misc_feature (1)...(7366): ZmCkx6 genomic sequence
aaaaactgtgcagctagctaagagaagctgaaaaacagtttttttttttaaaaaaaatctgtctactcttagagcatctccaacaacgtgacctataaaattgccctataatttgaaaataagtatattttatagaatttagggcaccaacaaaacacctcgctccaacagtaaagtcccaaatctagattatagggcagaccactacagtgtagtatatttgagtcacttgagagggtgctctatagttttttgacaaaaaattatgaaatatggcactgttggagtagtttttcctgtgtagagccctatatttcaattttaggcactagtttaaggcattgttggagatgctcttattttttaacgaaaagctgaaaaactggccttcgattgataaaaaaacattcagattaataatgttgtgagtggtacctatgccttctcttatttttttcttaatgatttatgagaaactataaattcttatattaacatatagagaaaaaggctctttgttttgcgaccgagcgagggagtatacacggatacaccggtacctccgctccgcacgtacctggaggctggagcagacgtttgactgggacgcgccgagtgtccggccaatgagagcgacgcacgtagcgcgggggcgccgctgcggcggcacatcatcacgtgcatgcggccacgcgcgcgggcgacagacaacgcgcgagcgacaggtcgacccccgtggccgaaccgaatcgcgtaggggatctcgacctatggcagcaaatttaacgccgcgttccggtggcggtcccgctccagcgatggccgcgtaccgtacctacggcgaccagaccacgggataatgcgtgcgattgttcttttgggtgggggagaatgctcgatcgatcgcaaatgccggtgctccccggccgttcgtcgtcggccggtcgatcacaggtacatactggcagtaaaaacagacgtgcaggttcccgacctgtcatcgtattatattcggcgttactgacaccatggcaatggcatgcatggtacgaagccaagtaaggagcagacgtgttcgtacgcctgtcgtcgtcttcgcgcgcgcgcccacgagcagcatgtctcacgcgcccagcaaattcgcgcgcgcggatgcagcccgatcggttatattcgatcggttataatgcatcatcgtcaacggcgtcaaaacaacgcgagagaggacacctacatttttcccctccggaaattaatcttaaaatttgcgcctcttatgctattaatatacgtattaaaatttgtataatttaaaactcaaaaaacattgccaaatgcattgacgcgattaaaaagttaaaaaaacaaaaaggataagaataagtgtagctacttttgaactttaaaacgtggtaaggctacagtgcagctacctttgtctagttactgcctcgtgcgtggaagattagaattccacctagagtacgtttttttccttctttttgttagttattactaacaataaagttctaactagagacaatttggctaattaaaagaaggaaagcagaggatgcaagctgcctgttctgtacagagcctgaatatgcacgtcatctctgaagttactaaccgtaatttaggagagaaaatatagcagagacaggaaaatcgttcggtgtatctggaaactcacgaatgagttatgttttcagagaaacttgctcgagaagcatggagctgttactacacacgcgataagcggactttcacagaaatggaaaactttacgcccgccagaaacgaaagagcaattggagatcagatcaccgtggagaaaaataatagcgtgtttggtttgtaggttgggctgcttctggagccatccagacctgtgtccgagcctacatcagcgtttggtttgaatcgcagaatgatgtcgtccgccactgtattgttctaataataaactagcatgcgggttcaactcactccacaaggaactgccggacggctccatccggagccaagccacgacggatgagcgaaaccgccggaccaaacgcgctgtaaaagaatgcagataggttaggttttgggagttgtgtgatcttcagctttctgccgataggctgtctgtaagaggtctttcagttttgtttggttctgtttctggttggaaccagttcccttggcctcaggcttcagcacaagtctaggtgtgatttaaactgcactgtattgaatactttagtcttttgacaatactgtagttaaaaggccggggggttttgccttggaactctaaaaaaatatacagtattaaccatggactctgaactctgtctgcgtccacaggcaagtcatctttcttccttgcactggttatcttattgaaacagaacggaaatcttttttggaacaagagaatttcgtcacatcttgcctgcagtaaagtttcccatctagatgcatactccctccgtccaagtttaactggcgttttagcttttctcagacataaataccagccaagagaatagacgcatgtacccctgtgatctagcgtgaagtattaattgcaattactgctgaggacacgaaacggttcacaacctccagccctccacggtggatgagaggagaccaagagtccgttggtgtgggaacgaagcgaacgggtgtgtgaaacgaggagataactataatggcatcgaggtgtagaccacgaacgacacataattctggacaaataaaatgagctaaaacgccattaaacttggacggagggagtatactatcaacatttcgatcaaaagttactatacaaaatttgcactgtccgaaaagcgatccttatcaggaaggcgcaggattcgtcccagctaagcgcaccggccacaagtattccaccacccccggtcaatagctaaagaaattgggcggcaagtgaaagtctccgggatgggaatgtgcatgagtcatgacgcgcctccgccctccggcctccgcagttgtttattcgcagcgcgcgggtggcggcccgcccgtccgtgttctctgctccctgtgttcggcacatcgtcacccccaccgtttcctgtgcctctctctcctatcttcctcggtctcctcccgtaatcctttgcctgataccccgctctaccaggccgccaccacctccctccaggctccagcagcctataaatacgcccgcgtcgcccaccaccgcacaccacttgaatactccatctcaacttcccttcctctcccgtgctgcgctgagctatatagctgctcctcgacctccaagaagcacgcgggcggagcccggagcgagtgattagtgaaaggcatagcataaggccgcccggccgggaagtggtggcaatgacgcggtgcctcatgttcacgctgctgttcctcgtctcctccctcatctccaccgtggggctccccgtcgagccgcccgcggagctcctgcagctgggcggcggggacgtcggcggcgggcgcctgagcgtcgacgcgtccgacatcgcggaggcgtcgcgcgacttcgggggcgtcgcccgcgccgagcccatggcggtgttccatccgcgcgcggccggcgacgtggcgggcctggtcggcgccgcgttccggtcggcgcgcggcttccgcgtctcggcgcggggccacggccactccatcagcggccaggcgcaggcggccggcggcgtggtcgtggacatgagccgcggccgcggccccggcgccgccgtggcgcgggcattgcccgtgcactcggcggcgctgggcgggcactacgtggacgtctggggcggcgagctgtgggtggacgtgctcaactggacgctgtcccacggcgggctggcgccgcggtcgtggacggactacctgtacctgtccgtgggcggcaccctctccaacgccggcatcagcgggcaggcgttccaccacgggccacagatcagcaatgtctacgagctcgacgtcgtcacaggtagcgtagcagctagctaggcgatcgagccggccgacgagtcgtagatgcaaggcgctgtctctggcgggccgatcgacgaatgaaccgactgactgacacacgtgcgctgtgcgttggcagggaagggagaggtggtgacctgctcggagacggagaacccggacctgttcttcggcgtcctgggcgggctgggccagttcggcatcatcacgcgggcgcgcatcgccctggagcgtgctcccaagagggtaagtaaacagcttaggcagcccacaactgcgttctcctcgctcccttctgctctctgtcgtgtctggacctcccttcaggcgcgcgccctgatggatcaccacctgcaccgattagatagcctttaatttccctcccgtgagtcgtgtctcgatcgtgtggcaccgggaaaggaacacagggcggggcgggcagtgcatctcctcccgtggcgtgtgtcaccggcctcgctttccaactaatgaccgacccagcacaccggccggccatgcaagtggcgagcgagcgcgctgctgatccgttgaggaaagcgcccctctgccgtccgctaataaaacggctgccacatgtgtagcgtccgaaaaaagatccacgcgtagtacgtgtacgtcttgataatcgccactgtagtatccgtgggtctatctataagcagggcccccagccgcccgggccagccatggccagtccgacacatacgtacgcgtcatccggtcaggtgcatggtccacggtgcccctgctcaacgattagccgccgccgccgtgtcgtcgtcatcgagtccgcctttttctttttgtttagctcgtgggatctttcgcaagattttgttcctctgctaattaatcggcctgtaatcttagtagcagctaagaattgacgggcggaatgcatgggtggtggtggtggtggtggttgcttgcaggttcggtggatccgggcgctctactccaacttcagcgagttcacggcggaccaggagcgcctcatctccctcggcagcggcggcggacgccggttcgactacgtggagggcttcgtcgtcgccgccgagggcctcatcaacaactggaggtcctccttcttctcgccgcagaaccccgtgaagctcacctcgctcaagcaccattccagcgtcctctactgcctcgaggtcaccaagaactacgacgacgaaaccgcagggtcggtcgaccaggtaggtactcggactcggagccttgcttttcagttactagccgtatgataacagtagcaggcagtgtactgtgtgtgctaatatttttcttcttctctccgttcaggacgtggatacgctgctgggcgagctgaacttcctccctggcacggtgttcactacggacctgccgtacgtggacttcctggaccgcgtgcacaaggcggagctgaagctgcgcgccaaggggatgtgggaggtgccgcacccgtggctcaacctcttcgtgccggcgtcccgcatcgccgacttcgaccgcggcgtcttccgtggcgtgctggggggccgcaccgccggcgccggcggccccgtcctcatctaccccatgaacaagcacaagtaagtcgtcaaatcacaaaggcacacacggaccacaagccaaaggccgcgcgcgctcatcgatctgccgccacgcacgcggaggcgcggcgcgtcgccctcgccgtccccgcccccgccccgcgcttccaaccaaccaaccttccattccattccatccacatgcggcgctcgggacacggggacaggcggacagcaccctccctcacatgcgccgcccacacgcggacggcaccgcacgcgcagcgcaggtgccggctcgctcatgtgtcatgtgttgcgattgcgatggacttgtgccgcccattattggcgccacgcacgcaaatcaccagccagcccatccgtagcagcggattattatttgcccaactgcgcgagccgaatctggagccccgatagatagaatggccgtggacaaaccgcgcactcgcgcgacagggcctactctattctgttgcaatggacgccacgcaactgcggccggtgacgccgacctgccatttataaaccggcgccgcgacgagagctttacagggcgcaacgagctcggagcggtcagaatcggaattccttagctgagacctgctcgccttttagtactttgtttgcattggggtcagtgggccgggtagacgcctagacggcaccaatttgtttgttgctttcagcttcagcgcatacacaaccaacctaaaaaaaagacgtatcagaatcaccagacgatcctgctaaaaaccgctgcttttttatttattttttcccgtactctattctgtactagtatcgtgcagtatcccaaacccttttcgcgtcgattagcgactgcagctctgagatctggactgggccgtgtggctgacgggctgcttcgtgcgcgcaggtgggacccgaggagctcggcggtgaccccggacgaggaggtgttctacctggtggcgttcctgcggtcggcgctgccgggcgcgccggagagcctggaggcgctggcgcggcagaaccagcggatcctcgacttctgcgcgggcgcgggcatcggcgccaagcagtacctgccgggccacaaggcgcggcacgagtgggcggagcacttcggcgccgcgaggtgggaccggttcgcgaggctcaaggccgagttcgacccgcgggccatcctggcggcggggcagggcatcttcaggccgcccggctcgccggcgctcgcggccgactcgtaacgtaatccagctgcttatactaattattaggcgcgtttagtgtaaggtagaggtactagctacagcagtaaccatacaggattgtttagttagctccggttggttcatgtacaaatgtggggttgttaatcgcgtgctctgccatggctgctgtgatcggttctgtacaggggatgaggggagccaaatatgaacgtggcaaaatcgatacttcttataaagaaaaaatatctatggtaaatactggtgcctgcctctgtttcccgtcaaactacagtgcagtgtattgttttctccgtggtccactggatataaggcattgcctgcaccttcatcctaaattataattcatttgactttttctgtcacgtttgaccgatcttcctatttatttattttttaaaataaatgaaaactcaaatataaagtatattatgtgctaaacaatattacgataaaaaaataacaataattatgatattttttaattcgaacgggttttccaagcacaaaaacgcatatatatgtatg

SEQ ID NO: 52 - DNA, 1623 bp, Zea mays
  CDS (1)...(1623)
atgacgcggtgcctcatgttcacgctgctgttcctcgtctcctccctcatctccaccgtggggctccccgtcgagccgcccgcggagctcctgcagctgggcggcggggacgtcggcggcgggcgcctgagcgtcgacgcgtccgacatcgcggaggcgtcgcgcgacttcgggggcgtcgcccgcgccgagcccatggcggtgttccatccgcgcgcggccggcgacgtggcgggcctggtcggcgccgcgttccggtcggcgcgcggcttccgcgtctcggcgcggggccacggccactccatcagcggccaggcgcaggcggccggcggcgtggtcgtggacatgagccgcggccgcggccccggcgccgccgtggcgcgggcattgcccgtgcactcggcggcgctgggcgggcactacgtggacgtctggggcggcgagctgtgggtggacgtgctcaactggacgctgtcccacggcgggctggcgccgcggtcgtggacggactacctgtacctgtccgtgggcggcaccctctccaacgccggcatcagcgggcaggcgttccaccacgggccacagatcagcaatgtctacgagctcgacgtcgtcacagggaagggagaggtggtgacctgctcggagacggagaacccggacctgttcttcggcgtcctgggcgggctgggccagttcggcatcatcacgcgggcgcgcatcgccctggagcgtgctcccaaggttcggtggatccgggcgctctactccaacttcagcgagttcacggcggaccaggagcgcctcatctccctcggcagcggcggcggacgccggttcgactacgtggagggcttcgtcgtcgccgccgagggcctcatcaacaactggaggtcctccttcttctcgccgcagaaccccgtgaagctcacctcgctcaagcaccattccagcgtcctctactgcctcgaggtcaccaagaactacgacgacgaaaccgcagggtcggtcgaccaggacgtggatacgctgctgggcgagctgaacttcctccctggcacggtgttcactacggacctgccgtacgtggacttcctggaccgcgtgcacaaggcggagctgaagctgcgcgccaaggggatgtgggaggtgccgcacccgtggctcaacctcttcgtgccggcgtcccgcatcgccgacttcgaccgcggcgtcttccgtggcgtgctggggggccgcaccgccggcgccggcggccccgtcctcatctaccccatgaacaagcacaagtgggacccgaggagctcggcggtgaccccggacgaggaggtgttctacctggtggcgttcctgcggtcggcgctgccgggcgcgccggagagcctggaggcgctggcgcggcagaaccagcggatcctcgacttctgcgcgggcgcgggcatcggcgccaagcagtacctgccgggccacaaggcgcggcacgagtgggcggagcacttcggcgccgcgaggtgggaccggttcgcgaggctcaaggccgagttcgacccgcgggccatcctggcggcggggcagggcatcttcaggccgcccggctcgccggcgctcgcggccgactcgtaa

SEQ ID NO: 53 - protein, 540 aa, Zea mays
MTRCLMFTLLFLVSSLISTVGLPVEPPAELLQLGGGDVGGGRLSVDASDIAEASRDFGGVARAEPMAVFHPRAAGDVAGLVGAAFRSARGFRVSARGHGHSISGQAQAAGGVVVDMSRGRGPGAAVARALPVHSAALGGHYVDVWGGELWVDVLNWTLSHGGLAPRSWTDYLYLSVGGTLSNAGISGQAFHHGPQISNVYELDVVTGKGEVVTCSETENPDLFFGVLGGLGQFGIITRARIALERAPKVRWIRALYSNFSEFTADQERLISLGSGGGRRFDYVEGFVVAAEGLINNWRSSFFSPQNPVKLTSLKHHSSVLYCLEVTKNYDDETAGSVDQDVDTLLGELNFLPGTVFTTDLPYVDFLDRVHKAELKLRAKGMWEVPHPWLNLFVPASRIADFDRGVFRGVLGGRTAGAGGPVLIYPMNKHKWDPRSSAVTPDEEVFYLVAFLRSALPGAPESLEALARQNQRILDFCAGAGIGAKQYLPGHKARHEWAEHFGAARWDRFARLKAEFDPRAILAAGQGIFRPPGSPALAADS

SEQ ID NO: 54 - DNA, 1617 bp, Zea mays
  misc_feature (1)...(1617): ZmCkx3 cds
atggcaagaaggactcgtttcgtggccatcgccgccctcctcacaagcttcctcaacgtcgcagccgggcattcccggccactgtccggtgccggcctcccgggcgatcttttcgggctgggcatcgcgtcgaggatccgcacggacagcaactcgacggcgaaggcggcgacggacttcggccagatggtgagggccgcgccggaggccgtgttccaccccgccacgccggccgacatcgccgcgctcgtccggttctccgccacgtcggcggcgccgttccccgttgcgccgcgcgggcagggccactcctggcgcggccaggcgctcgccccgggcggcgtcgtcgtggacatgggctcgctggggcgcggcccccgcatcaacgtgtccgccgtggccggcgcggagccgttcgtcgacgccggcggggagcagctgtgggtcgacgtcctccgcgccacgctgcgacacggcctggcgccccgcgtgtggaccgactacctccggctcaccgtcggcggcacgctctccaacgcgggaatcggcgggcaggcgttccgacacggtccgcagatcgccaacgtgcatgaactcgacgtcgtcacaggcacaggtgagatggtgacatgctccatggacgtgaactcggacctgttcatggcggctctaggcgggttaggccagttcggggtcataaccagagcacggatccggcttgagccggcgcccaagagggtgcgctgggttcgacttgcctacaccgacgtcgctactttcaccaaggatcaggagtttctcatatcaaaccgggctagccaagtcgggttcgactacgtcgaaggccaggtccagctcagccggtccttggtcgaaggccccaaatcaacacccttcttctccggcgccgatgttgctaggcttgctggactcgcgtccaggaccggacctgctgcaatctactacatcgaaggcgccatgtactacaccaaggacaccgccatatctgtggacaagaaaatgaaggcactcctggatcagctgagcttcgagccagggtttgcgttcaccaaggacgtgacgttcgtgcagttcctcgatcgggtgcgcgaggaggagagggtgctccggtcagccggcgcgtgggaggtgccgcacccatggctgaacctcttcgtcccacggtcgcgcatcctcgacttcgacgacggagtgttcaaggctctgctcaaggactccaacccagctgggatcatcctcatgtaccccatgaacaaggataggtgggacgaccggatgacagcgatgaccccagccacggacgacgacgacatgttctatgccgttagtttcctttggtcagcactgtccgcagacgacgtgccccagctcgagagatggaacaaggcagtgctggacttctgtgatcggtcaggaatagaatgcaagcagtacctgccacactacacatctcaagacgggtggcgacggcatttcggggcgaaatggagcaggatcgctgagctgaaggccagatatgaccctcgggcattgttgtcgccgggccagaggatttttccggtgccagtagaggcatctggcattgcttctgcctga

SEQ ID NO: 55 - DNA, 1566 bp, Zea mays
  misc_feature (1)...(1566): ZmCkx4 cds
atgatgctcgcgtacatggaccgcgcgacggcggccgccgagccagaggacgccggccgcgagcccgccaccatggcgggcgggtgcgcggcggcggcgacggatttcggcgggctggggagcgccatgcccgcggccgtggtccgcccggcgagcgcggacgacgtggccagcgccatccgcgcggcggcgctgacgccgcacctcaccgtggccgcccgcgggaacgggcactcggtggccggccaggccatggccgagggcgggctggtcctcgacatgcgctcgctcgcggcgccgtcccggcgcgcgcagatgcagctcgtcgtgcagtgccccgacggcggcggcggccgccgctgcttcgccgacgtccccggcggcgcgctctgggaggaggtgctccactgggccgtcgacaaccacgggctcgccccggcgtcctggacggactacctccgcctcaccgtgggcggcacgctctccaatggcggcgtcagcggccagtccttccgctacgggccccaggtgtccaacgtggccgagctcgaggtggtcaccggcgacggcgagcgccgcgtctgctcgccctcctcccacccggacctcttcttcgccgtgctcggcgggctcggccagtttggcgtcatcacgcgcgcccgcatcccgctccacagggcgcccaaggcggtgcggtggacgcgcgtggtgtacgcgagcatcgcggactacacggcggacgcggagtggctggtgacgcggccccccgacgcggcgttcgactacgtggagggcttcgcgttcgtgaacagcgacgaccccgtgaacggctggccgtccgtgcccatccccggcggcgcccgcttcgacccgtccctcctccccgccggcgccggccccgtcctctactgcctggaggtggccctgtaccagtacgcgcaccggcccgacgacgacgacgaggaggaccaggcggcggtgaccgtgagccggatgatggcgccgctcaagcacgtgcggggcctggagttcgcggcggacgtcgggtacgtggacttcctgtcccgcgtgaaccgggtggaggaggaggcccggcgcaacggcagctgggacgcgccgcacccgtggctcaacctcttcgtctccgcgcgcgacatcgccgacttcgaccgcgccgtcatcaagggcatgctcgccgacggcatcgacgggcccatgctcgtctaccctatgctcaagagcaagtgggaccccaacacgtcggtggcgctgccggagggcgaggtcttctacctggtggcgctgctgcggttctgccggagcggcgggccggcggtggacgagctggtggcgcagaacggcgccatcctccgcgcctgccgcgccaacggctacgactacaaggcctacttcccgagctaccgcggcgaggccgactgggcgcgccacttcggcgccgccaggtggaggcgcttcgtggaccgcaaggcccggtacgacccgctggcgatcctcgcgccgggccagaagatcttccctcgggtcccggcgtccgtcgccgtgtag

SEQ ID NO: 56 - protein, 173 aa, Artificial Sequence
  Consensus sequence of FAD domains of ZmCkx2, 3, 4, and 5
PAAVLXPASPXDIAALVRXXFSAXSXSPLTVAARGXGHSXXGQAQAPGGIVVDMRSLXRGXRXXXXXLXVHXXXGGEGRXXFXDXXGGXLWIEVLRXTLXKHGLAPRSWTDYLRLTVGGTLSNAGXSGQAFRHGPQXSNVXXLEVVTGRGEXVTCSPSXNSDLFXAXLGGLGQ